(19) United States
(12) Patent Application Publication
(10) Pub. No.: US 2009/0270380 A1
(43) Pub. Date: Oct. 29, 2009

(54) 3,4,5-Substituted Piperidines as Renin Inhibitors

(76) Inventors: Peter Herold, Basel (CH); Robert Mah, Muttenz (CH); Vincenzo Tschinke, Binningen (CH); Nathalie Jotterand, Basel (CH); Dirk Behnke, Grenzach-Wyhlen (DE); Aleksandar Stojanovic, Basel (CH); Stefan Stuz, Basel (CH); Michael Quirmbach, Basel (CH); Stjepan Jelakovic, Freiburg (DE)

Correspondence Address: WENDEROTH, LIND & PONACK, L.L.P., 1030 15th Street, N.W.,, Suite 400 East, Washington, DC, 20005-1503, US

(21) Appl. No.: 11/887,354

(22) PCT Filed: Mar. 30, 2006
(86) PCT No.: PCT/EP2006/061193

§ 371 Date: Sep. 28, 2007

(30) Foreign Application Priority Data
Mar. 31, 2005 (CH) 593/05
Jan. 19, 2006 (CH) 086/06

(51) Int. Cl. A61K 31/538; C07D 265/36; A61P 9/00; A61P 9/12; A61P 13/12

(52) U.S. Cl. 514/230.5; 544/105; 544/73

(57) ABSTRACT

The application relates to novel substituted piperidines of the general formula (II) in which R¹, R^{2′}, R^{2″}, R^{4′}, X, Z, m and n have the meanings defined in the description, to a process for the preparation thereof and to the use of these compounds as medicines, especially as renin inhibitors.

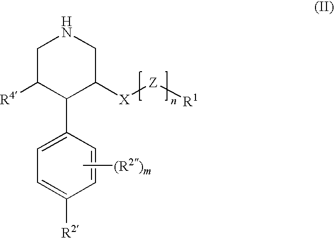

### FIELD OF THE INVENTION

The present invention relates to novel substituted piperidines, process for their preparation and the use of the compounds of medicines, especially as renin inhibitors.

### PRIOR ART

Piperidine derivatives for use as medicines are disclosed for example in WO 97/09311. However, in terms in particular of the renin inhibition, there continues to be a need for highly potent active ingredients. The priority in this connection is improving the pharmacokinetic properties. These properties, which are directed at better bioavailability, are for example absorption, metabolic stability, solubility or lipophilicity.

### DETAILED DESCRIPTION OF THE INVENTION

The invention therefore relates firstly to substituted piperidines of the general formula

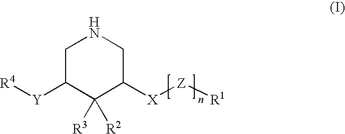

in which
(A) R¹ is aryl when R² is tetrazolyl or imidazolyl, each of which may be substituted by C₁₋₈alkoxy-C₁₋₈alkoxy-C₁₋₈alkyl, C₁₋₈alkoxy-C₁₋₈alkyl, aryloxy-C₁₋₈alkyl, heterocyclyloxy-C₁₋₈alkyl; or
(B) R¹ is aryl when X is O—CHR⁵—CO—NR⁶—; or
(C) R¹ is aryl when Z is -Alk-NR⁶—, where Alk is C₁₋₈alkylene, and n is 1; or
(D) R¹ is aryl which is substituted by 14 acetamidinyl-C₁₋₈alkyl, acyl-C₁₋₈alkoxy-C₁-alkyl, (N-acyl)C₁₋₈alkoxy-C₁₋₈alkylamino, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)C₁₋₈alkylaminocarbonyl-C₁₋₈alkoxy, (N—C₁-alkoxy)-C₁₋₈alkylaminocarbonyl-C₁₋₈alkyl, C₁₋₈alkoxy-C₁₋₁₈alkyl-carbamoyl, C₁₋₈alkoxy-C₁₋₈alkylcarbonyl, C₁₋₈alkoxy-C₁₋₈alkylcarbonylamino, 1-C₁₋₈alkoxy-C₁₋₈alkylimidazol-2-yl, 2-C₁₋₈alkoxy-C₁₋₈alkyl-4-oxoimidazol-1-yl, 1-C₁₋₈alkoxy-C₁₋₁₈alkyltetrazol-5-yl, 5-C₁₋₈alkoxy-C₁₋₈alkyltetrazol-1-yl, 6-alkoxy-aminocarbonyl-C₁₋₈alkoxy, C₁₋₈alkoxyaminocarbonyl-C₁₋₈alkyl, C₁₋₈alkoxycarbonyl, C₁₋₈alkoxycarbonyl-C₁₋₈alkoxy, C₁₋₈alkoxycarbonyl-C₁₋₈alkyl, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)-C₁₋₈alkoxy-C₁₋₈alkylcarbamoyl, (N—C₁₋₈alkyl)-C₁₋₈alkoxy-C₁₋₈alkylcarbonylamino, (N—C₁₋₈alkyl)-C₁₋₈alkoxycarbonylamino, (N—C₁₋₈alkyl)-C₀₋₈alkylcarbonylamino-C₁₋₈alkoxy, (N—C₁₋₈alkyl)C₀₋₈alkylcarbonylamino-C₁₋₈alkyl, (N—C₁₋₈alkyl)C₁₋₈alkylsulphonylamino-C₁₋₈alkoxy, (N—C₁₋₈alkyl)C₁₋₈alkylsulphonylamino-C₁₋₈alkyl, C₁₋₈alkylamidinyl, C₁₋₈alkyl-aminocarbonyl-C₁₋₈alkoxy, di-C₁₋₈alkylaminocarbonyl-C₁₋₈alkoxy, C₁₋₈alkylaminocarbonyl-C₁₋₈alkoxy-C₁₋₈alkyl, C₁₋₈alkylaminocarbonyl-C₁₋₈alkyl, C₁₋₈alkylaminocarbonylamino-C₁₋₈alkoxy, C₁₋₈alkylaminocarbonylamino-C₁₋₈alkyl, di-C₁₋₈alkylaminocarbonyl-C₁₋₈alkyl, C₁₋₈alkylamino-C₂₋₈alkoxy, di-C₁₋₈-alkylamino-C₂₋₈alkoxy, C₁₋₈alkylamino-C₁₋₈alkyl, di-C₁₋₈alkylamino-C₁₋₈alkyl, C₁₋₈alkylcarbamoyl, di-C₁₋₈alkylcarbamoyl, C₀₋₈alkylcarbonylamino-C₁₋₈alkoxy, C₀₋₈alkylcarbonylamino, C₀₋₈alkylcarbonylamino-C₁₋₈alkyl, C₁₋₈alkylcarbonyloxy-C₁₋₈alkoxy, C₁₋₈alkylcarbonyloxy-C₁₋₈alkyl, C₁₋₈alkylsulphonyl, C₁₋₈alkylsulphonyl-C₁₋₈alkoxy, C₁₋₈alkylsulphonyl-C₁₋₈alkyl, C₁₋₈alkylsulphonylamino-C₁₋₈alkoxy, C₁₋₈alkylsulphonylamino-C₁₋₈alkyl, carbamoyl, carbamoyl-C₁₋₈alkoxy, carbamoyl-C₁₋₈alkyl, carboxy-C₁₋₈alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkoxy, C₃₋₈cycloalkyl-C₁₋₈alkyl, C₃₋₈cycloalkylcarbonylamino-C₁₋₈alkoxy, C₃₋₈cycloalkylcarbonylamino-C₁₋₈alkyl, O,N-dimethylhydroxylamino-C₁₋₈alkyl, halogen, hydroxy-C₁₋₈alkoxy-C₁₋₈alkoxy, hydroxy-C₁₋₈alkoxy-C₁₋₈alkyl, hydroxy-C₁₋₈alkyl, (N-hydroxy)C₁-alkylaminocarbonyl-C₁₋₈alkoxy, (N-hydroxy)C₁₋₈alkylaminocarbonyl-C₁₋₈alkyl, (N-hydroxy)aminocarbonyl-C₁₋₈alkoxy, (N-hydroxy)aminocarbonyl-C₁₋₈alkyl, 2-oxoxazolidinyl-C₁₋₈alkoxy, 2-oxoxazolidinyl-C₁₋₈alkyl, O-methyloximyl-C₁₋₈alkyl or trifluoromethyl; or
(E) R¹ is aryl which is substituted by 1-4 3-acetamidomethylpyrrolidinyl, 3-C₁₋₈alkoxy-C₁₋₈alkylpyrrolidinyl, 3,4-dihydroxypyrrolidinyl, 2,6-dimethylmorpholinyl, 3,5-dimethylmorpholinyl, dioxanyl, dioxolanyl, 4,4-dioxothiomorpholinyl, dithianyl, dithiolanyl, 2-hydroxymethylpyrrolidinyl, 4-hydroxypiperidinyl, 3-hydroxypyrrolidinyl, imidazolylalkoxy, imidazolylalkyl, 2-methylimidazolylalkoxy, 2-methylimidazolylalkyl, 3-methyl-[1,2,4]-oxadiazol-5-ylalkoxy, 5-methyl-[1,2,4]-oxadiazol-3-ylalkoxy, 3-methyl-[1,2,4]-oxadiazol-5-ylalkyl, 5-methyl-[1,2,4]-oxadiazol-3-ylalkyl, 4-methylpiperazinyl, 5-methyltetrazol-1-ylalkoxy, 5-methyltetrazol-1-ylalkyl, morpholinyl, [1,2,4]-oxadiazol-5-ylalkoxy, [1,2,4]-oxadiazol-5-ylalkyl, oxazol-4-ylalkoxy, oxazol-4-ylalkyl, 2-oxo-[1,3]-oxazinyl, 2-oxoxazolidinyl, 2-oxoimidazolidinyl, 2-oxopyrrolidinyl, 4-oxopiperidinyl, 2-oxopyrrolidinylalkoxy, 2-oxopyrrolidinylalkyl, 2-oxotetrahydropyrimidinyl 4-oxo-thiomorpholinyl, piperazinyl, piperidinyl, pyrrolidinyl, pyrrolyl, [1,2,4]-triazol-1-yl-alkoxy, [1,2,4]-triazol-4-ylalkoxy, [1,2,4]-triazol-1-ylalkyl, [1,2,4]-triazol-4-ylalkyl, tetrazol-1-ylalkoxy, tetrazol-2-ylalkoxy, tetrazol-5-ylalkoxy, tetrazol-1-ylalkyl, tetrazol-2-ylalkyl, tetrazol-5-ylalkyl, thiazol-4-ylalkoxy, thiazol-4-ylalkyl or thiomorpholinyl; or
(F) R¹ is heterocyclyl optionally substituted by oxo or oxide or as indicated under (D) or (E), especially azepanyl, benzo[1,3]dioxolyl, benzofuranyl, benzoimidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydro-quinoxalinyl, 1,1a,2,7b-tetrahydrocyclopropa[c]chromenyl, tetrahydropyranyl or triazinyl;
   R² is phenyl, naphthyl, acenaphthyl, cyclohexyl, pyridyl, pyrimidinyl, pyrazinyl, oxopyridinyl, diazinyl, triazolyl, thienyl, oxazolyl, oxadiazolyl, thiazolyl, pyrrolyl, furyl, tetrazolyl or imidazolyl, where the radicals are substituted by 1-3 C₂₋₈alkenyloxy-C₁₋₈alkyl, C₁₋₈alkoxy-C₁₋₈alkyl, C₁₋₈alkoxy-C₁₋₈alkylamino-C₁₋₈alkyl, C₁₋₈alkoxy-C₁₋₈alkylsulphanyl-C₁₋₈alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₁₋₈alkylsulphonyl-C₁₋₈alkoxy-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, optionally halogen-substituted C₁₋₈alkoxy-C₁₋₈alkoxy-C₁₋₈alkyl, or (oxygen-heterocyclyl)-C₀₋₈alkoxy-C₁₋₈alkyl, and may, in addition to the aforementioned substituents, also be substituted by a maximum of 4 halogen;
   R³ is hydrogen, hydroxy, C₁₋₈alkoxy or C₂₋₈alkenyloxy;
   R⁴ is optionally halogen- and/or hydroxy-substituted C₁₋₈alkyl, optionally halogen- and/or hydroxy-substituted C₁₋₈alkoxy-C₁₋₈alkyl, optionally N-mono- or N,N-di-C₁₋₈alkylated amino-C₁₋₈alkyl, optionally N-mono- or N,N-di-C₁₋₈-alkylated or optionally hydroxy-substituted amino-C₀₋₈alkylcarbonyl-C₁₋₈alkyl, hydroxy-C₀₋₈alkylcarbonyl-C₀₋₈alkyl, C₁₋₈alkoxy-C₀₋₈alkylcarbonyl-C₀₋₈alkyl, optionally N—C₁₋₁₈-alkylated C₁₋₈alkoxycarbonylamino-C₁₋₈alkyl, optionally N—C₁₋₈-alkylated C₁₋₈alkoxy-C₁₋₈alkylamino-C₁₋₈alkyl, optionally N—C₁₋₈-alkylated or optionally halogen-substituted C₁₋₈alkylcarbonylamino-C₁₋₈alkyl, cyano-C₁₋₈alkyl, optionally N—C₁₋₈-alkylated or optionally halogen-substituted C₃₋₈-cycloalkyl-C₀₋₈alkylcarbonylamino-C₁₋₈alkyl, optionally N—C₁₋₈-alkylated hydroxy-C₁₋₈alkylamino-C₁₋₈alkyl, heterocyclylcarbonyl-C₀₋₈alkylamino-C₁₋₈alkyl, C₁₋₈alkoxycarbonylamino-C₁₋₈alkyl, optionally N—C₁₋₈-alkylated or optionally halogen-substituted heterocyclyl-C₀₋₈alkylcarbonylamino-C₁₋₈alkyl, C₃₋₈cycloalkyl-C₀₋₈alkyl, C₃₋₈cycloalkyloxy-C₁₋₈alkyl, heterocyclyl-C₀₋₈-(optionally hydroxy-substituted)alkyl, optionally N—C₁₋₈-alkylated heterocyclyl-C₀₋₈alkylamino-C₀₋₈alkylcarbonyl-C₀₋₈alkyl, C₁₋₈alkylsulphonyl-C₁₋₈alkyl, C₂₋₈alkinyl, heterocyclyl-C₂₋₈alkinyl, optionally N-mono- or N,N-di-C₁₋₈-alkylated amino-C₂₋₈alkinyl, N-mono- or N,N-di-C₁₋₈-alkylated aminocarbonyl-C₂₋₈alkinyl, heterocyclylcarbonyl-C₀₋₈alkyl or heterocyclyloxy-C₁₋₈alkyl; and where
   (a) if Y is —O— and R² is not para-C₁₋₈alkyl-substituted phenyl, then R⁴ may additionally also be hydrogen, and
   (b) if Y is oxo, then R⁴ is absent;
      R⁵ is acyl, C₂₋₈alkenyl, C₁₋₈alkyl, aryl-C₁₋₈alkyl or hydrogen;
      R⁶ is acyl, C₁₋₈alkoxy-C₁-alkyl, C₁₋₈alkyl or aryl-C₁₋₈alkyl or hydrogen;
      R⁷ is C₁₋₈alkoxycarbonyl-C₁₋₈alkyl, C₁₋₈alkyl, carboxy-C₁₋₈alkyl or hydrogen;
      X is a bond, oxygen or sulphur, where the bond originating from an oxygen or sulphur atom leads to a saturated C atom of the group Z, or is a group >CH—R⁵, >CHOR⁶, —O—CO—, >CO, >C═NOR⁷, —O—CHR⁵— or —O—CHR⁵—CO—NR⁶—;
      Y is —O—, oxo or a bond;
      Z is C₁₋₈alkylene, C₂₋₈alkenylene, hydroxy-C₁₋₈alkylidene, —O—, —S—, —O-Alk-, S-Alk-, -Alk-O—, -Alk-S— or -Alk-NR⁶—, where Alk is C₁₋₈alkylene; and where
   (a) if Z is —O-Alk- or —S-Alk-, then X is —CHR⁵—; and
   (b) if X is a bond, then Z is C₂₋₈alkenylene, -Alk-O— or -Alk-S—;

### n is 1, or, if X is —O—CO— or O—CHR⁵—CO—NR⁶—, is 0 or 1;

and the salts thereof, preferably the pharmaceutically acceptable salts thereof.

Preference is given to piperidines of the formula

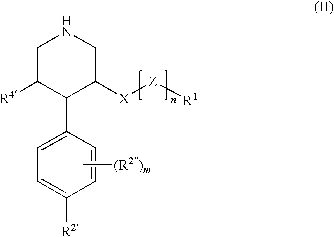

in which
(B) R¹ is aryl when X is —O—CHR⁵—CO—NR⁶—; or
(C) R¹ is aryl when Z is -Alk-NR⁶—, where Alk is C₁₋₈alkylene, and n is 1; or
(D) R¹ is aryl which is substituted by 1-4 acetamidinyl-C₁₋₈alkyl, acyl-C₁₋₈alkoxy-C₁₋₁₈alkyl, (N-acyl)C₁₋₈alkoxy-C₁₋₈alkylamino, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)C₁₋₈alkylaminocarbonyl-C₁₋₈alkoxy, (N—C₁₋₈alkoxy)—C₁₋₈alkylaminocarbonyl-C₁₋₈alkyl, C₁₋₈alkoxy-C₁₋₈alkyl-carbamoyl, C₁₋₈alkoxy-C₁₋₈alkylcarbonyl, C₁₋₈alkoxy-C₁₋₈alkylcarbonylamino, 1-C₁₋₈alkoxy-C₁₋₈alkylimidazol-2-yl, 2-C₁₋₈alkoxy-C₁₋₈alkyl-4-oxoimidazol-1-yl, 1-C₁₋₈alkoxy-C₁₋₈alkyltetrazol-5-yl, 5-C₁₋₈alkoxy-C₁₋₈alkyltetrazol-1-yl, 6-alkoxyamino-carbonyl-C₁₋₈alkoxy, C₁₋₈alkoxyaminocarbonyl-C₁₋₈alkyl, C₁₋₈alkoxycarbonyl, C₁₋₈alkoxycarbonyl-C₁₋₈alkoxy, C₁₋₈alkoxycarbonyl-C₁₋₈alkyl, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)C₁₋₈alkoxy-C₁₋₈alkylcarbamoyl, (N—C₁₋₈alkyl)C₁₋₈alkoxy-C₁₋₈alkylcarbonylamino, (N—C₁₋₈alkyl) C₁₋₈alkoxycarbonylamino, (N—C₁₋₈alkyl)-C₀₋₈alkylcarbonylamino-C₁₋₈alkoxy, (N—C₁₋₈-alkyl)-C₀₋₈alkylcarbonylamino-C₁₋₈alkyl, (N—C₁₋₈alkyl)C₁₋₈alkylsulphonylamino-C₁₋₈-alkoxy, (N—C₁₋₈-alkyl)-C₁₋₈alkylsulphonylamino-C₁₋₈alkyl, C₁₋₈alkylamidinyl, C₁₋₈alkylaminocarbonyl-C₁₋₈alkoxy, di-C₁₋₈alkylaminocarbonyl-C₁₋₈alkoxy, C₁₋₈alkyl-aminocarbonyl-C₁₋₈alkoxy-C₁₋₈alkyl, C₁₋₈alkylaminocarbonyl-C₁₋₈alkyl, C₁₋₈alkylamino-carbonylamino-C₁₋₈alkoxy, C₁₋₈alkylaminocarbonylamino-C₁₋₈alkyl, di-C₁₋₈alkyl-aminocarbonyl-C₁₋₈alkyl, C₁₋₈alkylamino-C₂₋₈alkoxy, di-C₁₋₈alkylamino-C₂₋₈alkoxy, C₁₋₈alkylamino-C₁₋₈alkyl, di-C₁₋₈alkylamino-C₁₋₈alkyl, C₁₋₈alkylcarbamoyl, di-C₁₋₈alkyl-carbamoyl, C₀₋₈alkylcarbonylamino-C₁₋₈alkoxy, C₀₋₈alkylcarbonylamino, C₀₋₈alkyl-carbonylamino-C₁₋₈alkyl, C₁₋₈alkylcarbonyloxy-C₁₋₈alkoxy, C₁₋₈alkylcarbonyloxy-C₁₋₈alkyl, C₁₋₈alkylsulphonyl, C₁₋₈alkylsulphonyl-C₁₋₈alkoxy, C₁₋₈alkylsulphonyl-C₁₋₈alkyl, C₁₋₈alkylsulphonylamino-C₁₋₈alkoxy, C₁₋₈alkylsulphonylamino-C₁₋₈alkyl, carbamoyl, carbamoyl-C₁₋₈alkoxy, carbamoyl-C₁₋₈alkyl, carboxy-C₁₋₄alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkoxy, C₃₋₈cycloalkyl-C₁₋₈alkyl, C₃₋₈cycloalkylcarbonylamino-C₁₋₈alkoxy, C₃₋₈cycloalkylcarbonylamino-C₁₋₈alkyl, O,N-dimethylhydroxylamino-C₁₋₈alkyl, halogen, hydroxy-C₁₋₈alkoxy-C₁₋₈alkoxy, hydroxy-C₁₋₈alkoxy-C₁₋₈alkyl, hydroxy-C₁₋₈alkyl, (N-hydroxy)C₁₋₈alkylaminocarbonyl-C₁₋₈alkoxy, (N-hydroxy)-C₁₋₈alkylaminocarbonyl-C₁₋₈alkyl, (N-hydroxy)aminocarbonyl-C₁₋₈alkoxy, (N-hydroxy)-aminocarbonyl-C₁₋₈alkyl, 2-oxoxazolidinyl-C₁₋₈alkoxy, 2-oxoxazolidinyl-C₁₋₁₈alkyl, O-methyloximyl-C₁₋₈alkyl or trifluoromethyl; or
(E) R¹ is aryl which is substituted by 1-4 3-acetamidomethylpyrrolidinyl, 3-C₁₋₈alkoxy-C₁₋₈alkylpyrrolidinyl, 3,4-dihydroxypyrrolidinyl, 2,6-dimethylmorpholinyl, 3,5-dimethyl-morpholinyl, dioxanyl, dioxolanyl, 4,4-dioxothiomorpholinyl, dithianyl, dithiolanyl, 2-hydroxymethylpyrrolidinyl, 4-hydroxypiperidinyl, 3-hydroxypyrrolidinyl, imidazolyl-alkoxy, imidazolylalkyl, 2-methylimidazolylalkoxy, 2-methylimidazolylalkyl, 3-methyl-[1,2,4]-oxadiazol-5-ylalkoxy, 5-methyl-[1,2,4]-oxadiazol-3-ylalkoxy, 3-methyl-[1,2,4]-oxadiazol-5-ylalkyl, 5-methyl-[1,2,4]-oxadiazol-3-ylalkyl, 4-methylpiperazinyl, 5-methyltetrazol-1-ylalkoxy, 5-methyltetrazol-1-ylalkyl, morpholinyl, [1,2,4]-oxadiazol-5-ylalkoxy, [1,2,4]-oxadiazol-5-ylalkyl, oxazol-4-ylalkoxy, oxazol-4-ylalkyl, 2-oxo-[1,3]-oxazinyl, 2-oxoxazolidinyl, 2-oxoimidazolidinyl, 2-oxopyrrolidinyl, 4-oxopiperidinyl, 2-oxopyrrolidinylalkoxy, 2-oxopyrrolidinylalkyl, 2-oxotetrahydropyrimidinyl 4-oxo-thiomorpholinyl, piperazinyl, piperidinyl, pyrrolidinyl, pyrrolyl, [1,2,4]-triazol-1-ylalkoxy, [1,2,4]-triazol-4-ylalkoxy, [1,2,4]-triazol-1-ylalkyl, [1,2,4]-triazol-4-ylalkyl, tetrazol-1-ylalkoxy, tetrazol-2-ylalkoxy, tetrazol-5-ylalkoxy, tetrazol-1-ylalkyl, tetrazol-2-ylalkyl, tetrazol-5-ylalkyl, thiazol-4-ylalkoxy, thiazol-4-ylalkyl or thiomorpholinyl; or
(F) R¹ is heterocyclyl, optionally substituted by oxo or oxide or as indicated under (D) or (E), especially azepanyl, benzo[1,3]dioxolyl, benzofuranyl, benzoimidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1A6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydrocyclopropa[c]chromenyl, tetrahydropyranyl or triazinyl;
   R²′ is C₂₋₈alkenyloxy-C₁₋₈alkyl, C₁₋₈alkoxy-C₁₋₈alkyl, C₁₋₈alkoxy-C₁₋₈alkylamino-C₁₋₈alkyl, C₁₋₈alkoxy-C₁₋₈alkylsulphanyl-C₁₋₈alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₁₋₈alkyl-sulphonyl-C₁₋₈alkoxy-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, optionally halogen-substituted C₁₋₈alkoxy-C₁₋₈alkoxy-C₁₋₈alkyl, or (oxygen-heterocyclyl)-C₀₋₈alkoxy-C₁₋₈alkyl;
   R²″ is halogen,
   R⁴′ is a) optionally halogen- and/or hydroxy-substituted C₁₋₈alkoxy, optionally halogen- and/or hydroxy-substituted-C₁₋₈alkoxy-C₁₋₈alkoxy, optionally N-mono- or N,N-di-C₁₋₈-alkylated amino-C₁₋₈alkoxy, optionally N—C₁₋₈-alkylated C₁₋₈alkoxy-C₁₋₈alkylamino-C₁₋₈alkoxy, optionally N-mono- or N,N-di-C₁₋₈-alkylated amino-C₀₋₈alkylcarbonyl-C₁₋₈alkoxy, hydroxy-C₀₋₈alkyl-carbonyl-C₀₋₈alkoxy, C₁₋₈alkoxy-C₀₋₈alkylcarbonyl-C₀₋₈alkoxy, C₁₋₈alkylcarbonylamino-C₁₋₈alkoxy, cyano-C₁₋₈alkoxy, C₃₋₈cycloalkyl-C₀₋₈alkoxy, heterocyclyl-C₀₋₈alkoxy, optionally N—C₁₋₈-alkylated heterocyclyl-C₀₋₈alkylamino-C₀₋₈alkylcarbonyl-C₀₋₈alkoxy, C₁₋₈alkylsulphonyl-C₁₋₈alkoxy, C₂₋₈alkinyloxy, heterocyclyl-C₂₋₈alkinyloxy, optionally N-mono- or N,N-di-C₁₋₆ alkylated amino-C₂₋₈alkinyloxy, N-mono- or N,N-di-C₁₋₈-alkylated aminocarbonyl-C₂₋₈alkinyl-oxy, heterocyclylcarbonyl-C>alkoxy, optionally N-mono- or N,N-di-C₁₋₈-alkylated amino-C₁₋₈alkyl, optionally N—C₁₋₈-alkylated C₁₋₈alkoxy-C₁₋₈alkylamino-C₁₋₈alkyl, optionally N-mono- or N,N-di-C₁₋₈-alkylated and optionally hydroxy-substituted amino-C₀₋₈alkylcarbonyl-C₀₋₈alkyl, optionally N—C₁₋₈-alkylated heterocyclyl-C₀₋₈alkylamino-C₀₋₈alkylcarbonyl-C₀₋₈alkyl, optionally halogen- or hydroxy-substituted C₁₋₈alkoxy-C₁₋₈alkyl, optionally halogen- and/or hydroxy-substituted C₁₋₈alkyl, optionally N—C₁₋₈-alkylated hydroxy-C₁₋₈alkylamino-C₁₋₈alkyl, heterocyclylcarbonyl-C₀₋₈alkyl, heterocyclylcarbonyl-C₀₋₈alkylamino-C₁₋₈alkyl, heterocyclyl-C₁₋₈alkyl, C₁₋₈alkoxycarbonylamino-C₁₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkyl-carbonylamino-C₁₋₈alkyl or optionally halogen-substituted C₁₋₈alkylcarbonylamino-C₁₋₈alkyl; or additionally
   b) is hydroxy if R²′ is not C₁₋₈alkyl;
      R⁵ is acyl, C₂₋₈alkenyl, C₁₋₈alkyl, aryl-C₁₋₈alkyl or hydrogen;
      R⁶ is acyl, C₁₋₈alkoxy-C₁₋₈alkyl, C₁₋₈alkyl or aryl-C₁₋₈alkyl or hydrogen;
      R⁷ is C₁₋₈alkoxycarbonyl-C₁₋₈alkyl, C₁₋₈alkyl, carboxy-C₁₋₈alkyl or hydrogen;
      X is a bond, oxygen or sulphur, where the bond originating from an oxygen or sulphur atom leads to a saturated C atom of the group Z, or is a group >CH—R⁵, >CHOR⁶, —O—CO—, >CO, >C═NOR⁷, —O—CHR⁵— or —CHR⁵—CO—NR⁶—;
      Z is C₁₋₈alkylene, C₂₋₈alkenylene, hydroxy-C₁₋₈alkylidene, —O—, —S—, —O-Alk-, —S-Alk-, -Alk-O—, -Alk-S— or -Alk-NR⁶—,
      where Alk is C₁₋₈alkylene; and where
   (a) if Z is —O-Alk- or —S-Alk-, then X is —CHR⁵—; and
   (b) if X is a bond, then Z is C₂₋₈alkenylene, -Alk-O— or -Alk-S—;
      m is 0, 1 or 2;

### n is 1 or, if X is —O—CO— or —O—CHR⁵—CO—NR⁸—, is 0 or 1;

and the salts thereof, preferably the pharmaceutically acceptable salts thereof.

Unless specified further, C₁₋₈alkyl and alkoxy radicals may be linear or branched. Examples of C₁₋₈alkyl and alkoxy radicals are methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₀alkoxy is —O— (oxygen). C₁₋₈alkylenedioxy radicals are preferably methylenedioxy, ethylenedioxy and propylenedioxy. Examples of C₁₋₈alkanoyl radicals are acetyl, propionyl and butyryl. Cycloalkyl is a saturated, cyclic hydrocarbon radical having 3 to 12 carbon atoms, for example cyclopropyl, cyclobutyl, cyclopentyl, cyclohexyl, cycloheptyl, bicyclo[2.2.1]heptyl, cyclooctyl, bicyclo[2.2.2]octyl and adamantyl. Cycloalkyl may be unsubstituted or substituted one or more times, e.g. substituted once or twice by C₁₋₈alkanoyl, C₂₋₈alkenyl, C₂₋₈alkinyl, C₁₋₈alkoxy, C₁₋₈alkoxy-C₁-alkoxy, C₁₋₈alkoxy-C₁₋₈alkyl, C₁₋₈alkoxycarbonylamino, C₁₋₈alkyl, C₀₋₈alkylcarbonylamino, C₁₋₈alkylcarbonyloxy, C₁₋₈alkylenedioxy, optionally N-mono- or N,N-di-C₁₋₈-alkylated amino, aryl, optionally N-mono- or N,N-di-C₁₋₈-alkylated carbamoyl, optionally esterified carboxy, cyano, C>cycloalkoxy, halogen, heteroaryl, heterocyclyl, hydroxy, oxo, polyhalo-C₁₋₈alkoxy or polyhalo-C₁₋₈alkyl. C₁₋₈Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₈alkenylene radicals are, for example, vinylene and propenylene; C₂₋₈alkinylene radicals is, for example, ethinylene; acyl radicals are alkanoyl radicals, preferably C₁₋₈alkanoyl radicals, or aroyl radicals such as benzoyl. Aryl refers to mono- or polynuclear aromatic radicals which may be substituted one or more times, such as, for example, phenyl, substituted phenyl, naphthyl, substituted naphthyl, tetrahydronaphthyl or substituted tetrahydronaphthyl. Examples of substituents on such aryl radicals are C₁₋₈alkyl, trifluoromethyl, nitro, amino, C₂₋₈alkenyl, C₁₋₈alkoxy, C₁₋₈alkylcarbonyloxy, hydroxy, halogen, cyano, carbamoyl, carboxy and C₁₋₈alkylenedioxy, and optionally halogen-, C₁₋₈alkyl-, C₁₋₈alkoxy- or dihydroxy-C₁₋₈alkyl-aminocarbonyl-substituted phenyl, phenoxy, phenylthio, phenyl-C₁₋₈alkyl or phenyl-C₁₋₈alkoxy. Further examples of substituents on aryl or heterocyclyl radicals are C₁₋₈alkoxy-carbonylphenyl, hydroxy-C₁₋₈alkylphenyl, benzyloxy, pyridylcarbonylamino-C₁₋₈alkyl, C₂₋₈alkenyloxy, C₁₋₈alkoxy-C₁₋₈alkoxy, C₁₋₈alkoxy-C₁₋₈alkoxy-C₁₋₈alkyl, methoxybenzyloxy, hydroxybenzyloxy, phenaethyloxy, methylenedioxybenzyloxy, dioxolanyl-C₁₋₈alkoxy, cyclopropyl-C₁₋₈alkyl, cyclopropyl-C₁₋₈alkoxy, hydroxy-C₁₋₈alkoxy, carbamoyloxy-C₁₋₈alkoxy, pyridylcarbamoyloxy-C₁₋₈alkoxy, benzoyloxy-C₁₋₈alkoxy, C₁₋₈alkoxycarbonyl, C₁₋₈alkyl-carbonylamino, C₀₋₈alkylcarbonylamino-C₁₋₈alkyl, C₁₋₈alkylcarbonylamino-C₁₋₈alkoxy, (N—C₁₋₈alkyl)-C₀₋₈alkylcarbonylamino-C₁₋₈alkyl, (N—C₁₋₈alkyl)-C₀₋₈alkylcarbonylamino-C₁₋₈alkoxy, C₃₋₈-cycloalkylcarbonylamino-C₁₋₈alkyl, C₃₋₈cycloalkylcarbonylamino-C₁₋₈alkoxy, C₁₋₈alkoxy-C₁₋₈alkyl, hydroxy-C₁₋₈alkyl, hydroxy-C₁₋₈alkoxy-C₁₋₈alkyl, hydroxy-C₁₋₈alkoxy-C₁₋₈alkoxy, C₁₋₈alkoxycarbonylamino-C₁₋₈alkyl, C₁₋₈alkoxycarbonylamino-C₁₋₈alkoxy, C₁₋₈alkylaminocarbonylamino-C₁₋₈alkyl, C₁₋₈alkylaminocarbonylamino-C₁₋₈alkoxy, C₁₋₈alkyl-aminocarbonyl-C₁₋₈alkyl, C₁₋₈alkylaminocarbonyl-C₁₋₈alkoxy, C₁₋₈alkylaminocarbonyl-C₁₋₈alkoxy-C₁₋₈alkyl, di-C₁₋₈alkylaminocarbonyl-C₁₋₈alkyl, di-C₁₋₈alkylaminocarbonyl-C₁₋₈alkoxy, C₁₋₈alkylcarbonyloxy-C₁₋₈alkyl, C₁₋₈alkylcarbonyloxy-C₁₋₈alkoxy, cyano-C₁₋₈alkyl, cyano-C₁₋₈alkoxy, 2-oxoxazolidinyl-C₁₋₈alkyl, 2-oxoxazolidinyl-C₁₋₈alkoxy, C₁₋₈alkoxycarbonyl-C₁₋₈alkyl, C₁₋₈alkoxycarbonyl-C₁₋₈alkoxy, C₁₋₈alkylsulphonylamino-C₁₋₈alkyl, C₁₋₈alkylsulphonyl-amino-C₁₋₈alkoxy, (N—C₁₋₈alkyl)C₁₋₈alkylsulphonylamino-C₁₋₈alkyl, (N—C₁₋₈alkyl)-C₁₋₈alkyl-sulphonylamino-C₁₋₈alkoxy, C₁₋₈alkylamino-C₁₋₈alkyl, C₁₋₈alkylamino-C₂₋₈alkoxy, di-C₁₋₈alkylamino-C₁₋₈alkyl, di-C₁₋₈alkylamino-C₂₋₈alkoxy, C₁₋₈alkylsulphonyl-C₁₋₈alkyl, C₁₋₈alkylsulphonyl-C₁₋₈alkoxy, carboxy-C₁₋₈alkyl, carboxy-C₁₋₈alkoxy, carboxy-C₁₋₈alkoxy-C₁₋₈alkyl, C₁₋₈alkoxy-C₁₋₈alkylcarbonyl, acyl-C₁₋₈alkoxy-C₁₋₈alkyl, (N—C₁₋₈alkyl)-C₁₋₈alkoxy-carbonylamino, (N-hydroxy)C₁-alkylaminocarbonyl-C₁₋₈alkyl, (N-hydroxy)-C₁₋₈alkylamino-carbonyl-C₁₋₈alkoxy, (N-hydroxy)aminocarbonyl-C₁₋₈alkyl, (N-hydroxy)aminocarbonyl-C₁₋₈alkoxy, C₁₋₈alkoxyaminocarbonyl-C₁₋₈alkyl, 6-alkoxyaminocarbonyl-C₁₋₈alkoxy, (N—C₁₋₈alkoxy)-C₁₋₈alkylaminocarbonyl-C₁₋₈alkyl, (N—C₁₋₈alkoxy)-C₁₋₈alkylaminocarbonyl-C₁₋₈alkoxy, (N-acyl)C₁₋₈alkoxy-C₁₋₈alkylamino, C₁₋₈alkoxy-C₁₋₈alkylcarbamoyl, (N—C₁₋₈alkyl)-C₁₋₈alkoxy-C₁₋₈alkylcarbamoyl, C₁₋₈alkoxy-C₁₋₈alkylcarbonyl, C₁₋₈alkoxy-C₁-alkylcarbonylamino, (N—C₁₋₈alkyl)C₁₋₈alkoxy-C₁₋₈alkylcarbonylamino, 1-C₁₋₈alkoxy-C₁₋₈alkylimidazol-2-yl, 1-C₁₋₈alkoxy-C₁₋₈alkyltetrazol-5-yl, 5-C₁₋₈alkoxy-C₁₋₈alkyltetrazol-1-yl, 2-C₁₋₈alkoxy-C₁₋₈alkyl-4-oxoimidazol-1-yl, carbamoyl-C₁₋₈alkyl, carbamoyl-C₁₋₈alkoxy, C₁₋₈alkylcarbamoyl, di-C₁₋₈alkylcarbamoyl, C₁₋₈alkylsulphonyl, C₁₋₈alkylamidinyl, acetamidinyl-C₁₋₈alkyl, O-methyloximyl-C₁₋₈alkyl, O,N-dimethylhydroxylamino-C₁₋₈alkyl, C₃₋₈cycloalkyl-C₁₋₈alkanoyl, aryl-C₁₋₈alkanoyl, heterocyclyl-C₁₋₈alkanoyl; and optionally halogen-, C₁₋₈alkyl-, C₁₋₈alkoxy- or dihydroxy-C₁₋₈alkylaminocarbonyl-substituted pyridyl, pyridyloxy, pyridylthio, pyridylamino, pyridyl-C₁₋₈alkyl, pyridyl-C₁₋₈alkoxy, pyrimidinyl, pyrimidinyloxy, pyrimidinylthio, pyrimidinyl-amino, pyrimidinyl-C₁₋₈alkyl, pyrimidinyl-C₁₋₈alkoxy, thienyl, thienyl-C₁₋₈alkyl, thienyl-C₁₋₈alkoxy, furyl, furyl-C₁₋₈alkyl, furyl-C₁₋₈alkoxy.

The term heterocyclyl refers to mono-, bi- or polycyclic, saturated and unsaturated hetero-cyclic radicals having 1 to 4 nitrogen and/or 1 or 2 sulphur or oxygen atoms (in case of oxygen referred to as oxygen-heterocyclyl), which may be substituted one or more times, in particular once, twice or three times. The term heterocyclyl further encompasses the above oxo-substituted radicals. Heterocyclyl radicals which comprise a nitrogen atom may be linked either via the N atom or via a C atom to the remainder of the molecule.

Examples of unsaturated heterocyclyl radicals are benzo[1,3]dioxolyl, benzofuranyl, benzoimidazolyl, benzooxazolyl, benzothiazolyl, benzo[b]thienyl, quinazolinyl, quinolyl, quinoxalinyl, 2H-chromenyl, dihydrobenzofuranyl, 1,3-dihydrobenzoimidazolyl, 3,4-dihydro-2H-benzo[1,4]oxazinyl, dihydro-3H-benzo[1,4]oxazinyl, 1,4-dihydrobenzo[d][1,3]oxazinyl, dihydro-2H-benzo[1,4]thiazinyl, 3,4-dihydro-1H-quinazolinyl, 3,4-dihydro-1H-quinolinyl, 2,3-dihydroindolyl, dihydro-1H-pyrido[2,3-b][1,4]oxazinyl, 1,1-dioxodihydro-2H-benzo[1,4]thiazinyl, furyl, imidazolyl, imidazo[1,5-a]pyridinyl, imidazo[1,2-a]pyrimidinyl, indazolyl, indolyl, isobenzofuranyl, isoquinolyl, [1,5]naphthyridyl, oxazolyl, 1-oxidopyridyl, 2-oxobenzoimidazolyl, 3-oxo-4H-benzo[1,4]oxazinyl, 2-oxobenzoxazolyl, 3-oxo-4H-benzo[1,4]thiazinyl, 2-oxo-1H-quinolinyl, 2-oxo-2H-chromenyl, 2-oxodihydrobenzo[e][1,4]-diazepinyl, 2-oxo-1,3-dihydrobenzoimidazole, 2-oxodihydrobenzo[d][1,3]oxazinyl, 2-oxo-3,4-dihydro-1H-quinazolinyl, 2-oxo-3,4-dihydro-1H-quinolinyl, 4-oxo-dihydroimidazolyl, 2-oxo-1,3-dihydroindolyl, 1-oxo-3H-isobenzofuranyl, 2-oxo-1H-pyrido[2,3-b][1,4]oxazinyl, 2-oxo-1,3,4,5-tetrahydrobenzo[b]azepinyl, 2-oxotetrahydrobenzo[e][1,4]diazepinyl, 4-oxo-3H-thieno[2,3-d]pyrimidinyl, 5-oxo-4H-[1,2,4]triazinyl, C₁₋₈alkylenedioxy-substituted phenyl, phthalazinyl, pyranyl, pyrazinyl, pyrazolyl, pyridyl, pyrimidinyl, 1H-pyrrolizinyl, pyrrolo[3,2c]pyridinyl, pyrrolo[2,3-c]pyridinyl, pyrrolo[3,2-b]pyridinyl, 1H-pyrrolo[2,3-b]pyridyl, pyrrolyl, 1,3,4,5-tetrahydrobenzo[b]azepinyl, tetrahydroquinolinyl, tetrahydroquinoxalinyl, tetrahydroisoquinolinyl, thiazolyl, thienyl, triazinyl, triazolyl, 1,1,3-trioxodihydro-2H-1λ6-benzo[1,4]thiazinyl, [1,2,3]triazolo[1,5-a]pyridinyl or [1,2,4]triazolo[4,3-a]pyridinyl.

The term saturated heterocyclyl refers to 3-16-membered, mono-, bi- or polycyclic saturated heterocyclic radicals having 1 to 4 nitrogen and/or 1 or 2 sulphur or oxygen atoms. Preference is given to 3-8-membered, particularly preferably 5- or 6-membered, monocyclic radicals which optionally have a 3-8-membered fused-on ring which may be carbocyclic or heterocyclic. A further preferred group of heterocyclic radicals are bi- or polycyclic heterocycles which optionally have a spirocyclic or bridged ring. Preferred heterocyclic radicals have in each ring 1 nitrogen, oxygen or sulphur atom, 1-2 nitrogen atoms and 1-2 oxygen atoms or 1-2 nitrogen atoms and 1-2 sulphur atoms, with at least 1, preferably 1-7, carbon atoms being present in each ring.

Examples of saturated heterocyclyl radicals are azepanyl, azetidinyl, aziridinyl, 3,4-dihydroxy-pyrrolidinyl, 2,6-dimethylmorpholinyl, 3,5-dimethylmorpholinyl, dioxanyl, [1,4]dioxepanyl, dioxolanyl, 4,4-di-oxothiomorpholinyl, dithianyl, dithiolanyl, 2-hydroxymethylpyrrolidinyl, 4-hydroxypiperidinyl, 3-hydroxypyrrolidinyl, 4-methylpiperazinyl, 1-methylpiperidinyl, 1-methylpyrrolidinyl, morpholinyl, oxathianyl, oxepanyl, 2-oxoazepanyl, 2-oxoimidazolidinyl, 2-oxooxazolidinyl, 2-oxopiperidinyl, 4-oxopiperidinyl, 2-oxopyrrolidinyl, 2-oxotetrahydro-pyrimidinyl, 4-oxothiomorpholinyl, piperazinyl, piperidinyl, pyrrolidinyl, tetrahydrofuranyl, tetrahydropyranyl, tetrahydrothiophenyl, tetrahydrothiopyranyl, thiepanyl or thiomorpholinyl.

Examples of bi- or polycyclic heterocyclyl radicals are 2,5-dioxabicyclo[4.1.0]heptanyl, 2-oxa-bicyclo[2.2.1]heptanyl, 2-oxabicyclo[4.1.0]heptanyl, 3-oxabicyclo[4.1.0]heptanyl, 7-oxa-bicyclo[2.2.1]heptanyl, 2-oxabicyclo[3.1.0]hexanyl, 3-oxabicyclo[3.1.0]hexanyl, 1-oxa-spiro[2.5]octanyl, 6-oxaspiro[2.5]octanyl, 3-oxabicyclo[3.3.1]nonanyl, 2-oxo-1a,7b-dihydro-1H-cyclopropa[c]chromenyl or 1,1a,2,7b-tetrahydrocyclopropa[c]chromenyl.

Heterocyclyl may be unsubstituted or substituted one or more times, e.g. once or twice, by C₁₋₈alkanoyl, C₂₋₈alkenyl, C₂₋₈alkinyl, C₁₋₈alkoxy, C₁₋₈alkoxy-C₁₋₈alkoxy, C₁₋₈alkoxy-C₁₋₈alkyl, C₁₋₈-alkoxycarbonylamino, C₁₋₈alkyl, C₀₋₈alkylcarbonylamino, C₁₋₈alkylcarbonyloxy, C₁₋₈-alkylenedioxy, optionally N-mono- or N,N-di-C₁₋₈-alkylated amino, aryl, optionally N-mono- or N,N-di-C₁₋₁₈-alkylated carbamoyl, optionally esterified carboxy, cyano, C₃₋₈cycloalkoxy, halogen, heteroaryl, heterocyclyl, hydroxy, nitro, oxide, oxo, polyhalo-C₁₋₈alkoxy or polyhalo-C₁₋₈alkyl.

The aryl, aroyl and heterocyclyl radicals in the case of R¹ may additionally be substituted also by heterocyclylalkyl, heterocyclylalkoxy, heterocyclylalkoxyalkyl or heterocyclyl such as, for example, piperidinoalkyl, piperidinoalkoxy, piperidinoalkoxyalkyl, morpholinoalkyl, morpholinoalkoxy, morpholinoalkoxyalkyl, piperazinoalkyl, piperazinoalkoxy, piperazino-alkoxyalkyl, [1,2,4]-triazol-1-ylalkyl, [1,2,4]-triazol-1-ylalkoxy, [1,2,4]-triazol-4-yl-alkyl, [1,2,4]-triazol-4-ylalkoxy, [1,2,4]-oxadiazol-5-ylalkyl, [1,2,4]-oxadiazol-5-ylalkoxy, 3-methyl-[1,2,4]-oxadiazol-5-ylalkyl, 3-methyl-[1,2,4]-oxadiazol-5-ylalkoxy, 5-methyl-[1,2,4]-oxadiazol-3-ylalkyl, 5-methyl-[1,2,4]-oxadiazol-3-ylalkoxy, tetrazol-1-ylalkyl, tetrazol-1-ylalkoxy, tetrazol-2-ylalkyl, tetrazol-2-ylalkoxy, tetrazol-5-ylalkyl, tetrazol-5-ylalkoxy, 5-methyl-tetrazol-1-ylalkyl, 5-methyl-tetrazol-1-ylalkoxy, thiazol-4-ylalkyl, thiazol-4-ylalkoxy, oxazol-4-ylalkyl, oxazol-4-ylalkoxy, 2-oxopyrrolidinylalkyl, 2-oxopyrrolidinylalkoxy, imidazolylalkyl, imidazolylalkoxy, 2-methylimidazolylalkyl, 2-methylimidazolylalkoxy or N-methylpiperazinoalkyl, N-methyl-piperazinoalkoxy, N-methylpiperazinoalkoxyalkyl, and alkylaminoalkyl, alkylaminoalkoxy, alkylaminoalkoxyalkyl, mono- and polyhydroxyalkyl, -alkoxy, -alkoxyalkyl and -alkoxyalkoxy, carbamoylalkyloxy, C₁₋₈alkoxy, amino-C₁₋₄alkoxy, hydroxy-C₁₋₈alkoxy, dioxolanyl, dioxanyl, dithiolanyl, dithianyl, pyrrolidinyl, piperidinyl, piperazinyl, pyrrolyl, 4-methylpiperazinyl, morpholinyl, thiomorpholinyl, 2-hydroxymethylpyrrolidinyl, 3-hydroxypyrrolidinyl, 3,4-dihydroxypyrrolidinyl, 3-acetamidomethylpyrrolidinyl, 3-C₁₋₈alkoxy-C₁₋₈alkylpyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, 2-oxoimidazolidinyl, 2-oxoxazolidinyl, 2-oxopyrrolidinyl, 2-oxo[1,3]oxazinyl, 2-oxo-tetrahydropyrimidinyl and the like or by the radical O—CH₂CH(OH)CH₂NRx, where NRx is a mono- or di-C₁₋₈alkylamino, piperidino, morpholino, piperazino or N-methylpiperazino radical.

The term polyhydroxyalkyl refers to C₁₋₈alkyl radicals which may be substituted by 2-8 hydroxy groups, such as, for example, glyceryl, arabityl, sorbityl etc.

The compounds of the formula (I) have at least two asymmetric carbon atoms, the compounds of the formula (II) have at least three asymmetric carbon atoms and may therefore exist in the form of optically pure diastereomers, mixtures of diastereomers, diastereomeric racemates, mixtures of diastereomeric racemates or as meso compounds. The invention encompasses all these forms. Mixtures of diastereomers, diastereomeric racemates or mixtures of diastereomeric racemates can be fractionated by conventional methods, e.g. by column chromatography, thin-layer chromatography, HPLC and the like. Salts of compounds with salt-forming groups are in particular acid addition salts, salts with bases or, if a plurality of salt-forming groups is present, optionally also mixed salts or inner salts.

Salts are primarily the pharmaceutically acceptable or non-toxic salts of compounds of the formulae (I) and (II).

Such salts are formed for example by compounds of the formula (I) and (II) having an acidic group, e.g. a carboxy or sulpho group, and are for example their salts with suitable bases, such as non-toxic metal salts derived from metals of group Ia, Ib, IIa and IIb of the Periodic Table of the Elements, e.g. alkali metal, in particular lithium, sodium or potassium, salts, alkaline earth metal salts, for example magnesium or calcium salts, furthermore zinc salts or ammonium salts, also salts formed with organic amines such as optionally hydroxy-substituted mono-, di- or trialkylamines, especially mono-, di- or tri-lower-alkylamines, or with quaternary ammonium bases, e.g. methyl-, ethyl-, diethyl- or triethylamine, mono-, bis- or tris(2-hydroxy-lower-alkyl)amines such as ethanol-, diethanol- or triethanolamine, tris(hydroxymethyl)methylamine or 2-hydroxy-tertiary-butylamine, N,N-di-lower-alkyl-N-(hydroxy-lower-alkyl)amine, such as N,N-dimethyl-N-(2-hydroxyethyl)amine, or N-methyl-D-glucamine, or quaternary ammonium hydroxides such as tetrabutylammonium hydroxide. The compounds of the formula I having a basic group, e.g. an amino group, can form acid addition salts, e.g. with suitable inorganic acids, e.g. hydrohalic acid such as hydrochloric acid, hydrobromic acid, sulphuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulphonic or phosphonic acids or N-substituted sulphamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, furthermore amino acids such as, for example, the α-amino acids mentioned herein-above, and methanesulphonic acid, ethanesulphonic acid, 2-hydroxyethanesulphonic acid, ethane-1,2-disulphonic acid, benzenesulphonic acid, 4-toluenesulphonic acid, naphthalene-2-sulphonic acid, 2- or 3-phosphoglycerate, glucose 6-phosphate, N-cyclohexylsulphamic acid (to form cyclamates) or with other acidic organic compounds such as ascorbic acid. Compounds of the formulae (I) and (II) having acidic and basic groups may also form inner salts.

Pharmaceutically unsuitable salts may also be used for isolation and purification.

The groups of compounds mentioned hereinafter are not to be regarded as closed; on the contrary, it is possible for parts of these groups of compounds to be interchanged or replaced by the definitions given above, or omitted, in a worthwhile manner, e.g. to replace general by more specific definitions. The definitions mentioned apply within the scope of general chemical principles such as, for example, the usual valencies of atoms.

Preferred compounds according to the invention are those of the general formula (IIA)

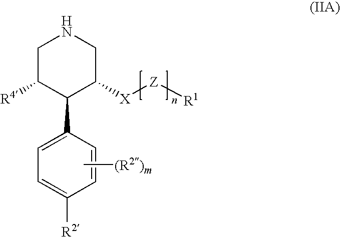

in which R¹, R²′, R²″, R⁴′, X and Z, and m and n have the meaning stated above for the compounds of the formula (II).

A further preferred group of compounds of the formula (II), and particularly preferably of the formula (IIA), are compounds in which

R¹ is aryl under the conditions as indicated for (B), (D) or (E), or is heterocyclyl, optionally substituted by oxo or oxide or as indicated under (D) or (E), where heterocyclyl is particularly preferably selected from azepanyl, benzo[1,3]dioxolyl, benzofuranyl, benzoimidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1λ6-benzo[1,4]thiazinyl, dihydro-1H-quinazolinyl, 1a,7b-dihydro-1H-cyclo-propa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydrocyclopropa[c]chromenyl, tetrahydropyranyl or triazinyl.

A further preferred group of compounds of the formula (II), and particularly preferably of the formula (IIA), are compounds in which

R¹ has the meaning as indicated for (B), (C), (D), (E) or (F), particularly preferably as indicated for (B), (D), (E) or (F);
R²′ is C₂₋₈alkenyloxy-C₁₋₈alkyl, C₁₋₈alkoxy-C₁₋₈alkyl, C₁₋₈alkoxy-C₁₋₈alkylamino-C₁₋₈alkyl, C₁₋₈alkoxy-C₁₋₈alkylsulphanyl-C₁₋₈alkyl, C₁₋₈alkoxy-C₁₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₁₋₈alkylsulphonyl-C₁₋₈alkoxy-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, optionally halogen-substituted C₁₋₈alkoxy-C₁₋₈alkoxy-C₁₋₈alkyl, or (oxygen-heterocyclyl)-C₀₋₈alkoxy-C₁₋₈alkyl;
R²″ is halogen;
R⁴′ has the meaning as indicated for (a) or (b);
R⁵ is acyl, C₂₋₈alkenyl, C₁₋₈alkyl, aryl-C₁-alkyl or hydrogen;
R⁶ is acyl, C₁₋₈alkoxy-C₁₋₈alkyl, C₁₋₈alkyl or aryl-C₁₋₈alkyl or hydrogen;
R⁷ is C₁₋₈alkoxycarbonyl-C₁₋₈alkyl, C₁₋₈alkyl, carboxy-C₁₋₈-alkyl or hydrogen;
X is a bond, oxygen or sulphur, where the bond originating from an oxygen or sulphur atom leads to a saturated C atom of the group Z, or is a group >CH—R⁵, >CHOR⁶, —O—CO—, >CO, >C═NOR⁷, —CHR⁵— or O—CHR⁵—CO—NR⁶—;
Z is C₁₋₈alkylene, C₂₋₈alkenylene, hydroxy-C₁₋₈alkylidene, —O—, —S—, —O-Alk-, —S-Alk-, -Alk-O—, -Alk-S— or -Alk-NR⁵—,
where Alk is C₁₋₈alkylene; and where
(a) if Z is —O-Alk- or —S-Alk-, then X is —CHR⁵—; and
(b) if X is a bond, then Z is C₂₋₈alkenylene, -Alk-O— or -Alk-S—;
   m is 0, 1 or 2;

### n is 1 or, if X is —CO— or —O—CHR⁵—CO—NR⁶—, is 0 or 1;

and pharmaceutically acceptable salts thereof.

Preference is furthermore given to compounds of the formulae (II) and (IIa) in which

X is preferably a bond, oxygen, sulphur, —O—CHR⁵— or —CO—.
Z is preferably methylene, —O—CHR⁵—CO—NR⁶— or -Alk-O—.

A group of preferred radicals R⁴′ includes

R⁴′ is a) optionally halogen and/or hydroxy-substituted C₁₋₈alkoxy, optionally halogen- and/or hydroxy-substituted C₁₋₈alkoxy-C₁₋₈alkoxy, optionally halogen-substituted hydroxy-C₁₋₈alkoxy, optionally N-mono- or N,N-di-C₁₋₈alkylated amino-C₁₋₈alkoxy, optionally N-mono- or N,N-di-C₁₋₈-alkylated amino-C₀₋₈alkylcarbonyl-C₁₋₈alkoxy, C₁₋₈alkoxy-C₀₋₈alkylcarbonyl-C₀₋₈alkoxy, cyano-C₁₋₈alkoxy, C₁₋₈cycloalkyl-C₀₋₈alkoxy, heterocyclyl-C>8alkoxy, C₁₋₈alkylsulphonyl-C₁₋₈alkoxy, C₂₋₈alkinyloxy, heterocyclyl-C₂₋₈alkinyloxy, optionally N-mono- or N,N-di-C₁-alkylated amino-C₂₋₈alkinyloxy, heterocyclylcarbonyl-C alkoxy, optionally N-mono- or N,N-di-C₁₋₈-alkylated amino-C₁₋₈alkyl, optionally N-mono- or N,N-di-C₁₋₈-alkylated and optionally hydroxy-substituted amino-C₀₋₈alkylcarbonyl-C₀₋₈alkyl, optionally halogen- or hydroxy-substituted C₁₋₈alkoxy-C₁₋₈-alkyl, optionally halogen- and/or hydroxy-substituted C₁₋₈alkyl, heteracyclyl-C₀₋₈alkylcarbonyl-C₁₋₈alkyl, optionally halogen-substituted heterocyclyl-C₀₋₈alkylcarbonylamino-C₁-alkyl, optionally halogen-substituted C₃₋₈cycloalkyl-C₀₋₈alkyl-carbonylamino-C₁₋₈alkyl or optionally halogen-substituted C₁₋₈alkylcarbonylamino-C₁₋₈alkyl; or additionally
b) is hydroxy if R²′ is not C₁₋₈alkyl;

A group of preferred radicals R¹ includes the abovementioned substituted phenyl and naphthyl radicals, and tetrahydronaphthyl and methyl-substituted tetrahydronaphthyl.

Likewise preferred radicals R¹ are azepanyl, benzo[1,3]dioxolyl, benzofuranyl, benzo-imidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1A6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydrocyclopropa[c]-chromenyl, tetrahydropyranyl or triazinyl, and azepanyl, benzo[1,3]dioxolyl, benzofuranyl, benzoimidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1A6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydrocyclo-propa[c]chromenyl, tetrahydropyranyl or triazinyl, each of which is substituted by 1-3 acetamidinyl-C₁₋₈alkyl, 3-acetamidomethylpyrrolidinyl, acyl-C₁₋₈alkoxy-C₁₋₈alkyl, (N-acyl)-C₁₋₈alkoxy-C₁₋₈alkylamino, C₁₋₈alkanoyl, C₁₋₈alkanoyloxy, C₂₋₈alkenyl, C₂₋₈alkenyloxy, C₂₋₈alkenyloxy-C₁₋₈alkyl, C₁₋₈alkoxy, C₁₋₈alkoxy-C₁₋₈-alkoxy, C₁₋₈alkoxy-C₁₋₈alkoxy-C₁₋₈alkyl, C₁₋₈alkoxy-C₁₋₈alkyl, (N—C₁₋₈alkoxy, C₁₋₈alkylaminocarbonyl-C₁₋₈alkoxy, (N—C₁₋₈alkoxy) C₁₋₈alkylaminocarbonyl-C₁₋₈alkyl, C₁₋₈alkoxy-C₁₋₈alkylcarbamoyl, C₁₋₈alkoxy-C₁₋₈alkylcarbonyl, C₁₋₈alkoxy-C₁₋₈alkylcarbonylamino, 1-C₁₋₈alkoxy-C₁₋₈alkylimidazol-2-yl, 2-C₁₋₈alkoxy-C₁₋₈alkyl-4-oxo-imidazol-1-yl, 3-C₁₋₈alkoxy-C₁₋₈alkylpyrrolidinyl, 1-C₁₋₈alkoxy-C₁₋₈alkyltetrazol-5-yl, 5-C₁₋₈alkoxy-C₁₋₈alkyltetrazol-1-yl, C₁₋₈alkoxyaminocarbonyl-C₁₋₈alkoxy, C₁₋₈alkoxyamino-carbonyl-C₁₋₈alkyl, C₁₋₈alkoxycarbonyl, C₁₋₈alkoxycarbonyl-C₁₋₈alkoxy, C₁₋₈alkoxycarbonyl-C₁₋₈alkyl, C₁₋₈alkoxycarbonylamino, C₁₋₈alkoxycarbonylamino-C₁₋₈alkoxy, C₁₋₈alkoxycarbonyl-amino-C₁₋₈alkyl, C₁₋₈alkyl, (N—C₁₋₈alkyl)C₁₋₈alkoxy-C₁₋₈alkylcarbamoyl, (N—C₁₋₈alkyl)-C₁₋₈alkoxy-C₁₋₈alkylcarbonylamino, (N—C₁₋₈alkyl)-C₁₋₈alkoxycarbonylamino, (N—C₁₋₈alkyl)-C₀₋₆alkylcarbonylamino-C₁₋₈alkoxy, (N—C₁₋₈alkyl)C₀₋₆alkylcarbonylamino-C₁₋₈alkyl, (N—C₁₋₈alkyl)-C₁₋₈alkylsulphonylamino-C₁₋₈alkoxy, (N—C₁₋₈alkyl)-C₁₋₈alkylsulphonylamino-C₁₋₈alkyl, C₁₋₈alkylamidinyl, C₁₋₈alkylamino, Di-C₁₋₈alkylamino, C₁₋₈alkylamino-C₂₋₈alkoxy, di-C₁₋₈alkylamino-C₂₋₈alkoxy, C₁₋₈alkylamino-C₁₋₈alkyl, C₁₋₈alkylaminocarbonyl, C₁₋₈alkyl-aminocarbonyl-C₁₋₈alkoxy, di-C₁₋₈alkylaminocarbonyl-C₁₋₈alkoxy, C₁₋₈alkylaminocarbonyl-C₁₋₈alkoxy-C₁₋₈alkyl, C₁₋₈alkylaminocarbonyl-C₁₋₈alkyl, C₁₋₈alkylaminocarbonylamino-C₁₋₈alkoxy, C₁₋₈alkylaminocarbonylamino-C₁₋₈alkyl, di-C₁₋₈alkylaminocarbonyl-C₁₋₈alkyl, di-C₁₋₈alkylamino-C₁₋₈alkyl, C₁₋₈alkylcarbamoyl, di-C₁₋₈alkylcarbamoyl, C₀₋₆alkylcarbonylamino, C₀₋₆alkylcarbonylamino-C₁₋₈alkoxy, C>alkylcarbonylamino-C₁₋₈alkyl, C₁₋₈alkylcarbonyloxy-C₁₋₈alkoxy, C₁₋₈alkylcarbonyloxy-C₁₋₈alkyl, C₁₋₈alkylendioxy, C₁₋₈alkylsulphonyl, C₁₋₄alkyl-sulphonyl-C₁₋₈alkoxy, C₁₋₈alkylsulphonyl-C₁₋₈alkyl, C₁₋₈alkylsulphonylamino-C₁₋₈alkoxy, C₁₋₈alkylsulphonylamino-C₁₋₈alkyl, amino, amino-C₂₋₇alkoxy, amino-C₁₋₈alkyl, aryl-C₁₋₈alkanoyl, benzoyloxy-C₂₋₈alkoxy, carbamoyl, carbamoyl-C₁₋₈alkoxy, carbamoyl-C₁₋₈alkyl, carboxy, carboxy-C₁₋₈alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkanoyl, C₃₋₈cycloalkyl-C₀₋₆alkoxy, C₃₋₈cyclo-alkyl-C₀₋₆alkyl, C₃₋₈cycloalkylcarbonylamino, C₃₋₈cycloalkylcarbonylamino-C₁₋₈alkoxy, C₃₋₈cycloalkylcarbonylamino-C₁₋₈-alkyl, 3,4-dihydroxypyrrolidinyl, O,N-dimethylhydroxylamino-C₁₋₈alkyl, 2,6-dimethylmorpholinyl, 3,5-dimethylmorpholinyl, dioxanyl, dioxolanyl, dioxolanyl-C₁₋₈alkoxy, 4,4-dioxothiomorpholinyl, dithianyl, dithiolanyl, optionally C₁₋₈alkoxy, C₁₋₈alkyl, dihydroxy-C₁₋₈alkylaminocarbonyl or halogen-substituted furyl, furyl-C₁₋₈alkoxy, furyl-C₁₋₈alkyl, pyridyl, pyridyl-C₁₋₈alkoxy, pyridyl-C₁₋₈alkyl, pyridylamino, pyridyloxy, pyridylthio, pyrimidinyl, pyrimidinyl-C₁₋₈alkoxy, pyrimidinyl-C₁₋₈alkyl, pyrimidinylamino, pyrimidinyloxy, pyrimidinylthio, thienyl, thienyl-C₁₋₈alkoxy or thienyl-C₁₋₈alkyl, halogen, heterocyclyl-C₁₋₈alkanoyl, hydroxy, hydroxy-C₂₋₈alkoxy, hydroxy-C₂₋₈alkoxy-C₁₋₈alkoxy, hydroxy-C₂₋₈alkoxy-C₁₋₈alkyl, hydroxy-C₁₋₈alkyl, (N-hydroxy)-C₁₋₈alkylaminocarbonyl-C₁₋₈alkoxy, (N-hydroxy)C₁₋₈alkylamino-carbonyl-C₁₋₈alkyl, (N-hydroxy)aminocarbonyl-C₁₋₈alkoxy, (N-hydroxy)aminocarbonyl-C₁₋₈alkyl, hydroxybenzyloxy, 2-hydroxymethylpyrrolidinyl, 4-hydroxypiperidinyl, 3-hydroxy-pyrrolidinyl, imidazolyl-C₁₋₈alkoxy, imidazolyl-C₁₋₈alkyl, methoxybenzyloxy, methylenedioxy-benzyloxy, 2-methylimidazolyl-C₁₋₈alkoxy, 2-methylimidazolyl-C₁₋₈alkyl, 3-methyl-[1,2,4]-oxadiazol-5-yl-C₁₋₈alkoxy, 5-methyl-[1,2,4]-oxadiazol-3-yl-C₁₋₈alkoxy, 3-methyl-[1,2,4]-oxadiazol-5-yl-C₁₋₈alkyl, 5-methyl-[1,2,4]-oxadiazol-3-yl-C₁₋₈alkyl, O-methyloximyl-C₁₋₈alkyl, 4-methylpiperazinyl, N-methylpiperazino-C₁₋₈alkoxy, N-methylpiperazino-C₁₋₈alkoxy-C₁₋₈alkyl, N-methylpiperazino-C₁₋₈alkyl, 5-methyltetrazol-1-yl-C₁₋₈alkoxy, 5-methyltetrazol-1-yl-C₁₋₈alkyl, morpholinyl, morpholino-C₁₋₈alkoxy, morpholino-C₁₋₈alkoxy-C₁₋₈alkyl, morpholino-C₁₋₈alkyl, nitro, [1,2,4]-oxadiazol-5-yl-C₁₋₈alkoxy, [1,2,4]-oxadiazol-5-yl-C₁₋₈alkyl, oxazol-4-yl-C₁₋₈alkoxy, oxazol-4-yl-C₁₋₈alkyl, oxide, oxo, 2-oxoimidazolidinyl, 2-oxo[1,3]oxazinyl, 2-oxoxazolidinyl, 2-oxoxazolidinyl-C₁₋₈alkoxy, 2-oxoxazolidinyl-C₁₋₈alkyl, 4-oxopiperidinyl, 2-oxopyrrolidinyl, 2-oxopyrrolidinyl-C₁₋₈alkoxy, 2-oxopyrrolidinyl-C₁-alkyl, 2-oxotetrahydropyrimidinyl, 4-oxo-thiomorpholinyl, optionally C₁₋₈alkoxy-, C₁₋₈alkoxycarbonyl-, C₁₋₈alkyl-, C₁₋₈alkylamino-, di-C₁₋₈alkylamino-, halogen-, hydroxy-, hydroxy-C₁₋₈alkyl- or trifluoromethyl-substituted phenoxy, phenyl, phenyl-C₁₋₈alkoxy, phenyl-C₁₋₈alkyl or phenylthio, piperazinyl, piperazino-C₁₋₈alkoxy, piperazino-C₁₋₈alkoxy-C₁₋₈alkyl, piperazino-C₁₋₈alkyl, piperidinyl, piperidino-C₁₋₈alkoxy, piperidino-C₁₋₈alkoxy-C₁₋₈alkyl, polyhalo-C₁₋₈-alkoxy, polyhalo-C₁₋₈alkyl, pyridylcarbamoyloxy-C₁₋₈alkoxy, pyridylcarbonylamino-C₁₋₈alkyl, pyrrolidinyl, pyrrolyl, tetrazol-1-yl-C₁₋₈alkoxy, tetrazol-2-yl-C₁₋₈alkoxy, tetrazol-5-yl-C₁₋₈alkoxy, tetrazol-1-yl-C₁₋₈alkyl, tetrazol-2-yl-C₁₋₈alkyl, tetrazol-5-yl-C₁₋₈alkyl, thiazol-4-yl-C₁₋₈alkoxy, thiazol-4-yl-C₁₋₈alkyl, thiomorpholinyl, [1,2,4]-triazol-1-yl-C₁₋₈alkoxy, [1,2,4]-triazol-4-yl-C₁₋₈alkoxy, [1,2,4]-triazol-1-yl-C₁₋₈alkyl, [1,2,4]-triazol-4-yl-C₁₋₈alkyl and the radical —O—CH₂CH(OH)CH₂NRx, where NRx is a mono- or di-C₁₋₈alkylamino, N-methylpiperazino, morpholino, piperazino or piperidino radical.

R¹ is very particularly preferably optionally substituted benzimidazolyl or a substituted radical selected from 2H-chromenyl, 3,4-dihydro-2H-benzo[1,4]oxazinyl, 1a,7b-dihydro-1H-cyclo-propa[c]chromenyl, indazolyl, indolyl, phenyl and 1,1a,2,7b-tetrahydrocyclopropa[c]chromenyl.

Preference is furthermore given to compounds of the formulae (II) and (IIA) in which

R²′ is C₁₋₈alkyl and
R⁴′ is optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈-alkylated amino-C₁₋₈alkoxy, heterocyclyl-C₀₋₈alkoxy, optionally N-mono- or N,N-di-C₁₋₈-alkylated amino-C₀₋₈alkylcarbonyl-C₁₋₈alkoxy, optionally N-mono- or N,N-di-C₁₋₈-alkylated and optionally hydroxy-substituted amino-C₀₋₈alkylcarbonyl-C₀₋₈alkyl, heterocyclylcarbonyl-C₀₋₈alkoxy, heterocyclyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkyl-carbonylamino-C₁₋₈alkyl or optionally halogen-substituted C₁₋₈alkylcarbonylamino-C₁₋₈alkyl;
and compounds of the formulae (II) and (IIA) in which
R²′ is C₁₋₈alkoxy-C₁₋₈alkyl and
R⁴′ is hydroxy, optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈-alkylated amino-C₁₋₈alkoxy, heterocyclyl-C₀₋₈alkoxy, optionally N-mono- or N,N-di-C₁₋₈-alkylated amino-C₀₋₈alkylcarbonyl-C₁₋₈alkoxy, heterocyclylcarbonyl-C₀₋₈alkoxy optionally N-mono- or N,N-di-C₁₋₈alkylated and optionally hydroxy-substituted amino-C₀₋₈alkylcarbonyl-C₀₋₈alkyl, heterocyclylcarbonyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkyl-carbonylamino-C₁₋₈alkyl or optionally halogen-substituted C₁₋₈alkylcarbonylamino-C₁₋₈alkyl.

Very particular preference is given to compounds of the formulae (II) and (IIA) in which

R¹ is optionally substituted benzoimidazolyl or a substituted radical selected from 2H-chromenyl, 3,4-dihydro-2H-benzo[1,4]oxazinyl, 1a, 7b-dihydro-1H-cyclopropa[c]chromenyl, indazolyl, indolyl, phenyl and 1,1a,2,7b-tetrahydrocyclopropa[c]chromenyl;
R²′ is C₂₋₈alkenyloxy-C₁₋₈alkyl, C₁₋₈alkoxy-C₁₋₈alkyl, C₁₋₈alkoxy-C₁₋₈alkylsulphanyl-C₁₋₈alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, or optionally halogen-substituted C₁₋₈alkoxy-C₁₋₈alkoxy-C₁₋₈alkyl;
R⁴′ is hydroxy, optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈-alkylated amino-C₁₋₈alkoxy, heterocyclyl-C₀₋₈alkoxy, optionally N-mono- or N,N-di-C₁₋₈-alkylated amino-C₀₋₈alkylcarbonyl-C₁₋₈alkoxy, heterocyclylcarbonyl-C₀₋₈alkoxy, optionally N-mono- or N,N-di-C₁₋₈-alkylated and optionally hydroxy-substituted amino-C₀₋₈alkylcarbonyl-C₀₋₈alkyl, heterocyclyl-C₀₋₈alkylcarbonyl-C alkyl, optionally halogen-substituted heterocyclyl-C₀₋₈alkyl-carbonylamino-C₁₋₆alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl;

### X is —O— or >CH—R⁵;

Z is C₁₋₈alkylene;
m is 0;
and
n is 1.

The compounds of the formula (I) and (II) can be prepared in an analogous manner to preparation processes disclosed in the literature. Similar preparation processes are described for example in WO 97/09311. Details of the specific preparation variants can be found in the examples.

The compounds of the formula (I) and (II) can also be prepared in optically pure form. Separation into antipodes can take place by methods known per se, either preferably at an early stage in the synthesis by salt formation with an optically active acid such as, for example, (+) or (−)-mandelic acid and separation of the diastereomeric salts by fractional crystallization or preferably at a rather late stage by derivatizing with a chiral auxiliary component such as, for example, (+) or (−)-camphanoyl chloride, and separation of the diastereomeric products by chromatography and/or crystallization and subsequent cleavage of the linkage to the chiral auxiliary. The pure diastereomeric salts and derivatives can be analysed to determine the absolute configuration of the contained piperidine by conventional spectroscopic methods, with X-ray spectroscopy on single crystals representing a particularly suitable method.

The compounds of the formula (I), (II) and (IIA) also include compounds in which one or more atoms are replaced by their stable, non-radioactive isotopes; for example a hydrogen atom by deuterium.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower ω-(amino, mono- or dialkylamino, carboxy, lower alkoxycarbonyl)—alkyl esters or such as lower α-(alkanoyloxy, alkoxycarbonyl or dialkylaminocarbonyl)—alkyl esters; conventionally, pivaloyloxymethyl esters and similar esters are used as such.

Because of the dose relationship between a free compound, a prodrug derivative and a salt compound, a particular compound in this invention also includes its prodrug derivative and salt form, where this is possible and appropriate.

The compounds of the formula (I), (II) or (IIA), and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octa-peptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radio-immunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/l.

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulphonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The compounds of the formula (I), and preferably of the formula (II) and (IIA), and their pharmaceutically acceptable salts can be used as medicines, e.g. in the form of pharmaceutical products. The pharmaceutical products can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I), or preferably of the formula (II) and (IIA), and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical products may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I), or preferably of the formula (II) and (IIA), and their pharmaceutically acceptable salts in the treatment or prevention of high blood pressure, heart failure, glaucoma, myocardial infarction, renal failure, restenoses and stroke.

The compounds of the formula (I), and preferably of the formula (II) and (IIA), and their pharmaceutically acceptable salts can also be administered in combination with one or more agents having cardiovascular activity, e.g. α- and β-blockers such as phentolamine, phenoxybenzamine, prazosin, terazosin, tolazine, atenolol, metoprolol, nadolol, propranolol timolol, carteolol etc.; vasodilators such as hydralazine, minoxidil, diazoxide, nitroprusside, flosequinan etc.; calcium antagonists such as aminone, bencyclan, diltiazem, fendiline, flunarizine, nicardipine, nimodipine, perhexyline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of the formulae (I), (II) or (IIA) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3g, preferably about 10 mg to about 1g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at room temperature. The abbreviation “Rf=xx (A)” means for example that the Rf is found in solvent system A to be xx. The ratio of amounts of solvents to one another is always stated in parts by volume. Chemical names for final products and intermediates have been generated with the aid of the AutoNom 2000 (Automatic Nomenclature) program.

| | | | R_{f} | Rt |
|---|---|---|---|---|
| No. | Structure | Appearance | (System) | (Method) |
| 4 | 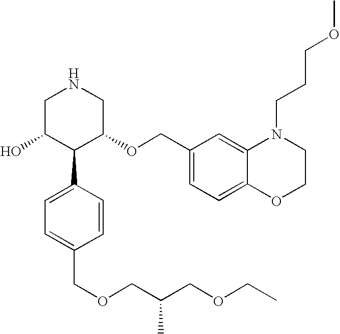 | yellowish resin | 0.21 (A) | 3.87 (I) |
| 5 | 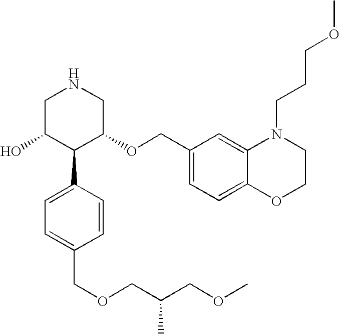 | colourless resin | 0.22 (A) | 3.66 (I) |
| 7 | 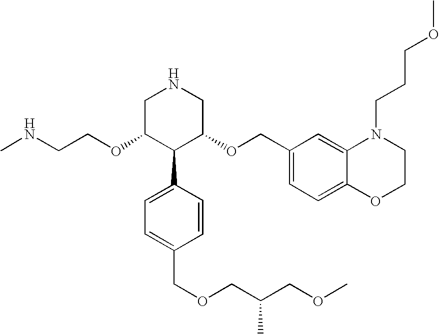 | colourless oil | 0.06 (D) | 3.30 (I) |
| 8 | 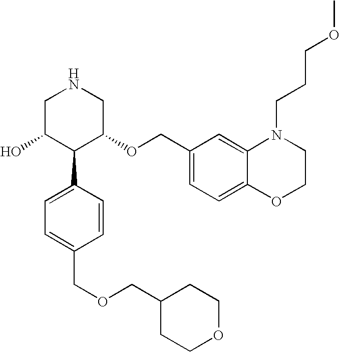 | colourless oil | 0.16 (A) | 3.83 (I) |
| 9 | 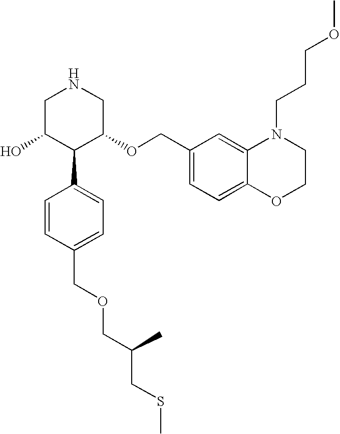 | yellowish resin | 0.10 (C) | 4.16 (I) |
| 10 | 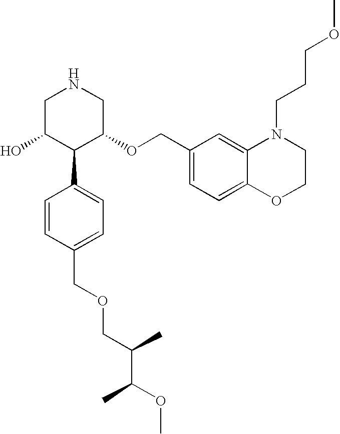 | yellowish oil | 0.33 (A) | 3.75 |
| 11 | 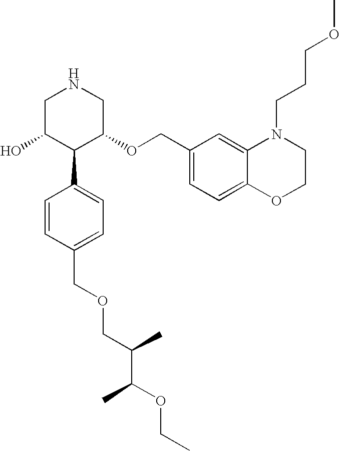 | yellowish oil | 0.33 (A) | 3.99 |
| 12 | 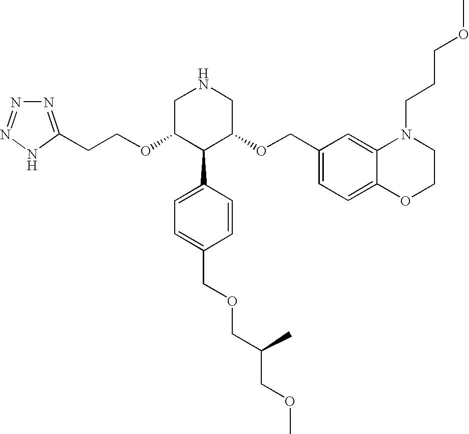 | beige solid | 0.1 (E) | 3.60 |
| 13 | 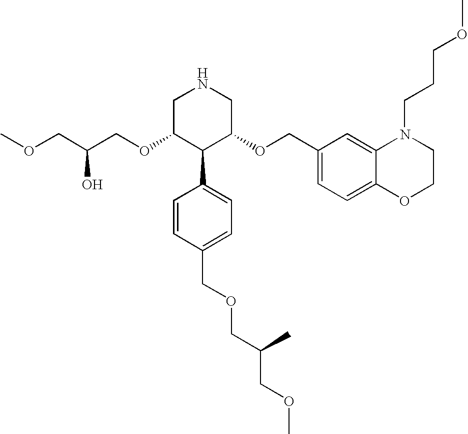 | yellowish oil | 0.35 (A) | 3.73 |
| 14 | 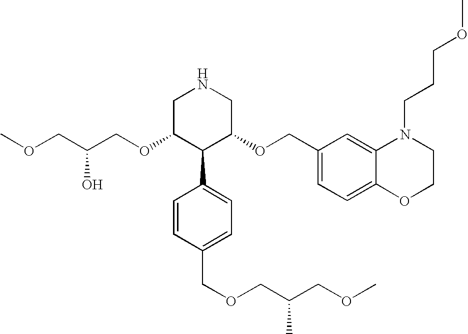 | yellowish oil | 0.35 (A) | 3.75 |
| 15 | 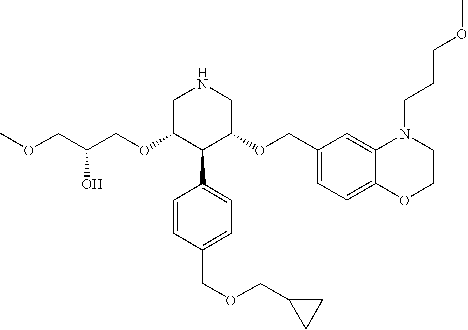 | colourless oil | 0.24 (A) | 3.76 |
| 16 | 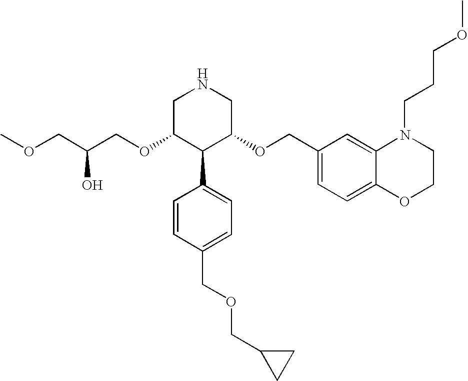 | yellowish oil | 0.24 (A) | 3.73 |
| 17 | 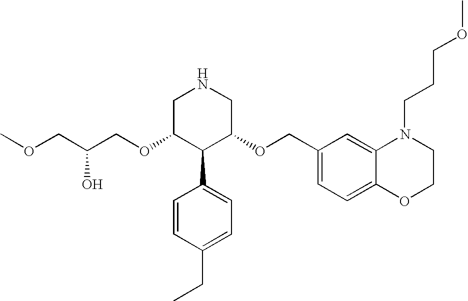 | yellowish oil | 0.19 (A) | 3.78 |
| 18 | 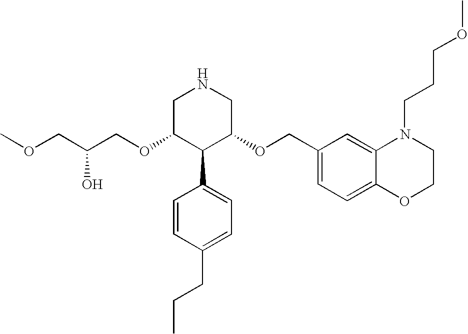 | yellowish oil | 0.17 (C) | 4.00 |
| 19 | 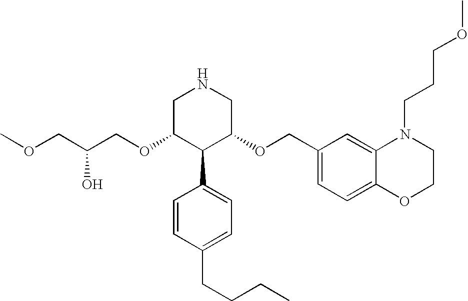 | yellowish oil | 0.17 (C) | 4.23 |
| 20 | 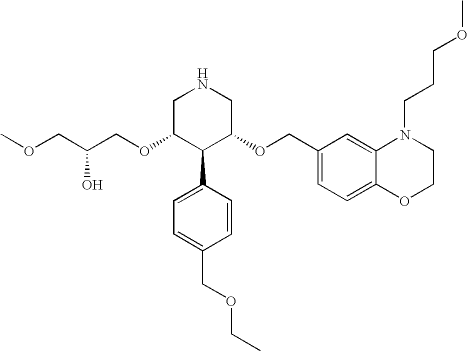 | yellowish oil | 0.13 (A) | 3.51 |
| 21 | 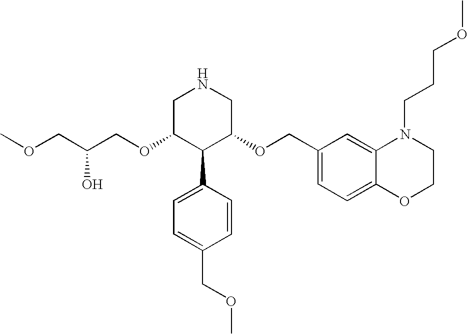 | yellow oil | 0.35 (G) | 3.32 |
| 22 | 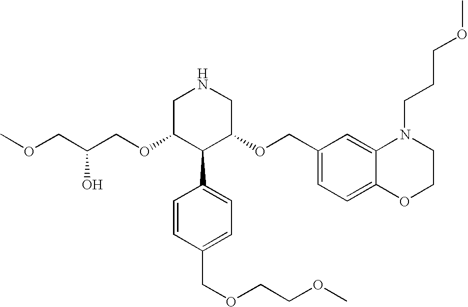 | yellow oil | 0.21 (A) | 3.29 |
| 23 | 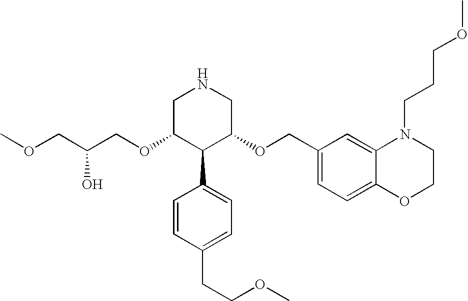 | yellowish oil | 0.11 (C) | 3.42 |
| 24 | 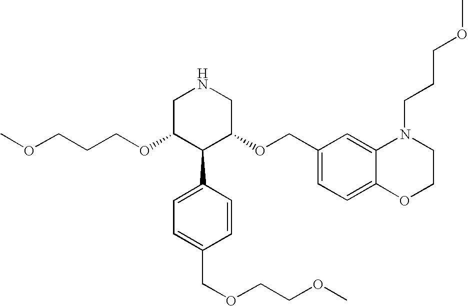 | yellow oil | 0.3 (A) | 3.58 |
| 25 | 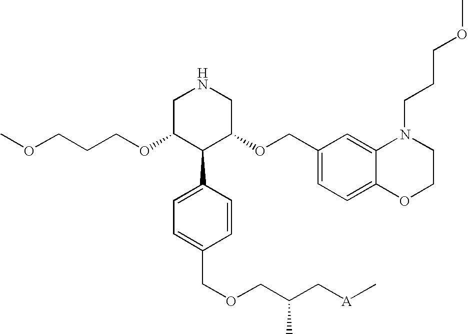 | yellow oil | 0.41 (A) | 4.03 |
| 26 | 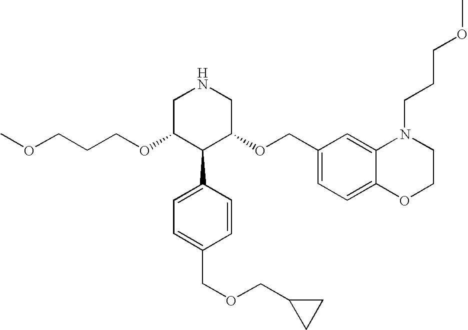 | yellow oil | 0.38 (A) | 4.14 |
| 27 | 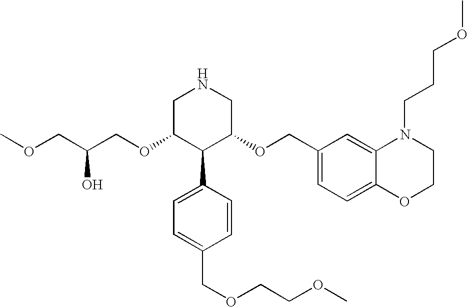 | yellow oil | 0.5 (F) | 3.28 |
| 28 | 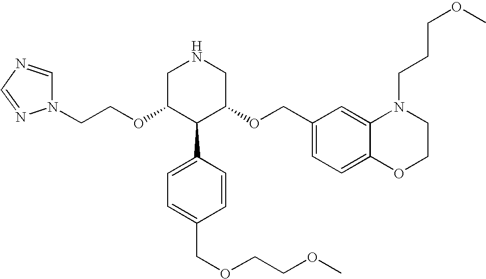 | yellowish oil | 0.28 (A) | 3.11 |
| 29 | 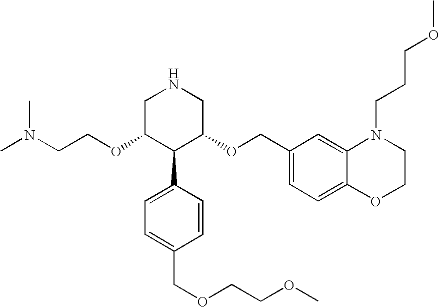 | yellowish oil | 0.11(A) | 2.99 |
| 30 | 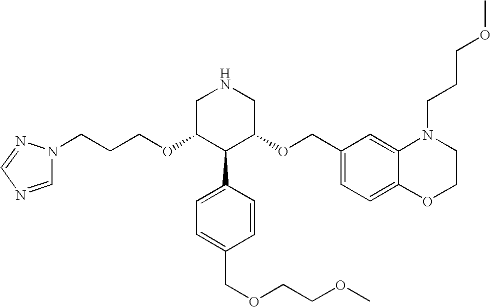 | yellowish oil | 0.28 (A) | 3.22 |
| 31 | 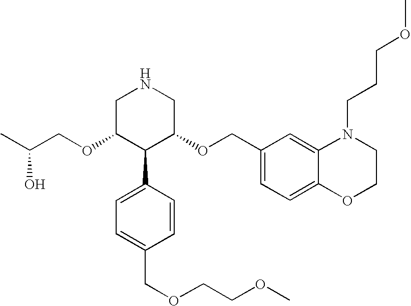 | colourless oil | 0.21 (A) | 3.32 |
| 32 | 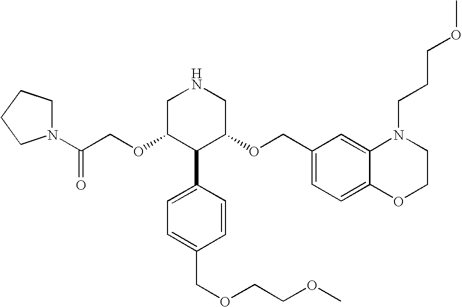 | colourless oil | 0.25 (A) | 3.46 |
| 33 | 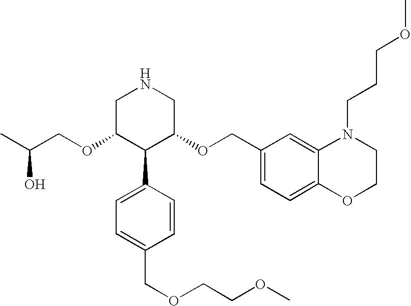 | pale yellow resin | 0.306 (A) | 3.30 |
| 34 | 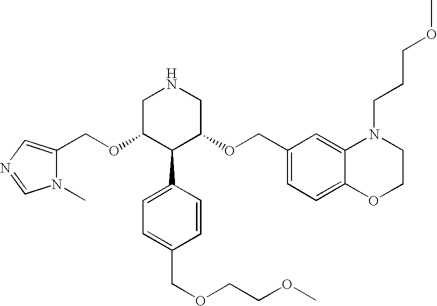 | yellowish oil | 0.18 (A) | 3.02 |
| 35 | 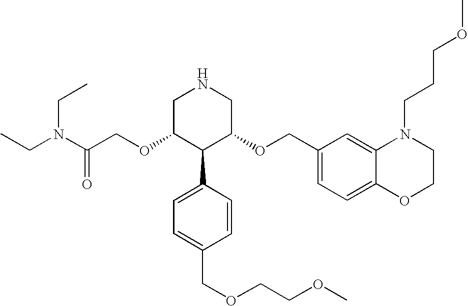 | yellow oil | 0.27 (A) | 3.64 |
| 36 | 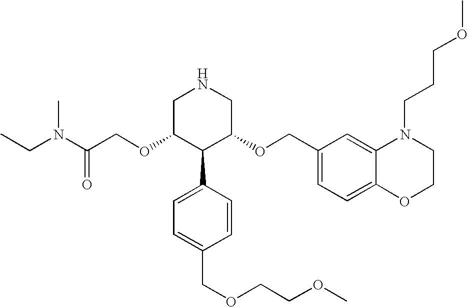 | yellow oil | 0.3 (D) | 3.47 |
| 39 | 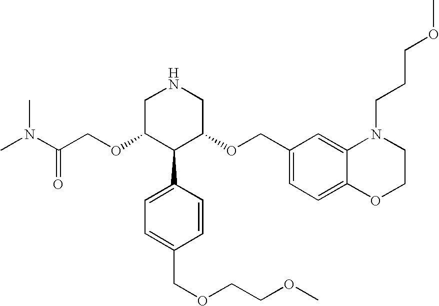 | yellowish oil | 0.29 (A) | 3.35 (I) |
| 40 | 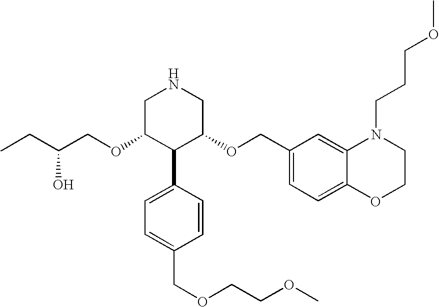 | pale yellow resin | 0.14 (A) | 3.43 (I) |
| 41 | 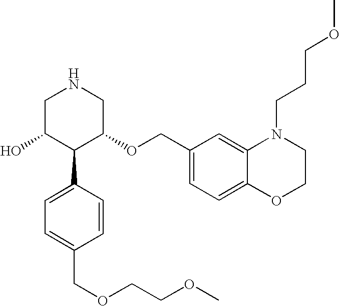 | yellowish resin | 0.18 (A) | 3.22 (I) |
| 42 | 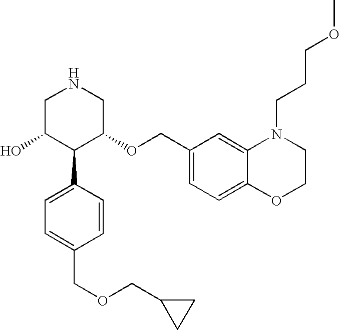 | yellow oil | 0.25 (A) | 3.64 (I) |
| 43 | 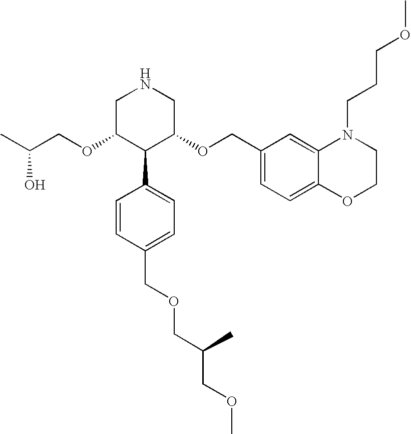 | yellowish resin | 0.42 (A) | 3.77 (I) |
| 44 | 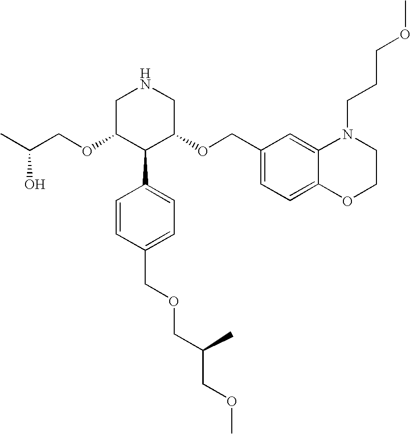 | yellowish resin | 0.47 (A) | 4.02 (I) |
| 45 | 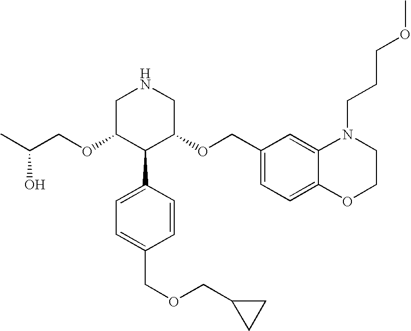 | pale yellow oil | 0.20 (A) | 3.76 (I) |
| 47 | 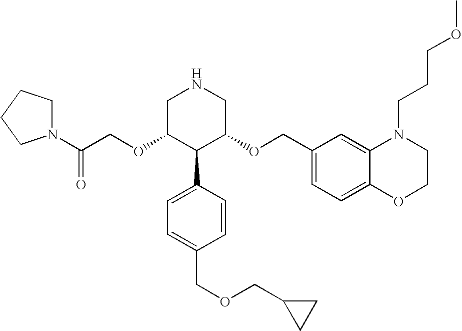 | yellowish oil | 0.23 (A) | 3.97 (I) |
| 48 | 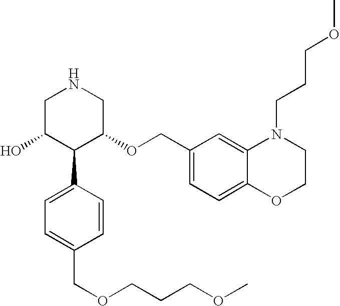 | yellowish oil | 0.20 (A) | 3.30 (I) |
| 49 | 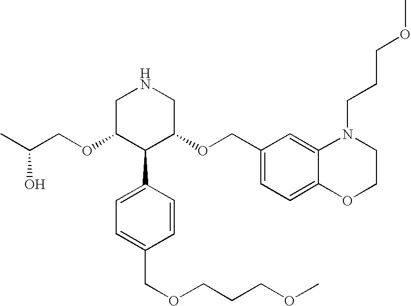 | yellowish oil | 0.20 (A) | 3.44 (I) |
| 52 | 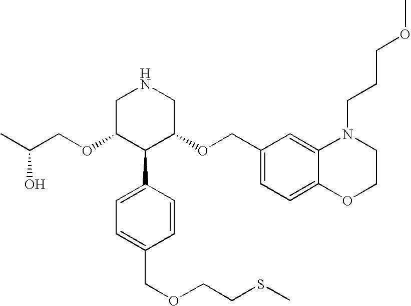 | yellowish oil | 0.40 (A) | 3.72 (I) |
| 57 | 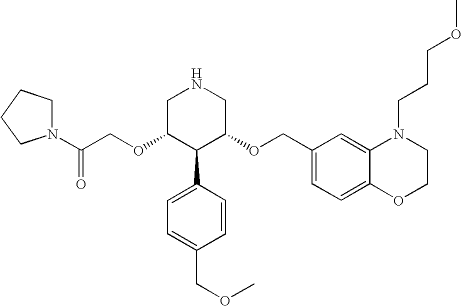 | yellowish oil | 0.24 (A) | 3.54 (I) |
| 59 | 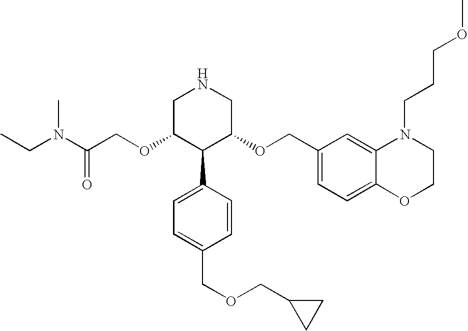 | yellowish oil | 0.28 (A) | 4.02 (I) |
| 60 | 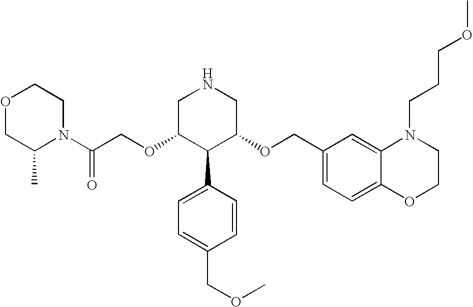 | yellowish oil | 0.31 (A) | 3.48 (I) |
| 61 | 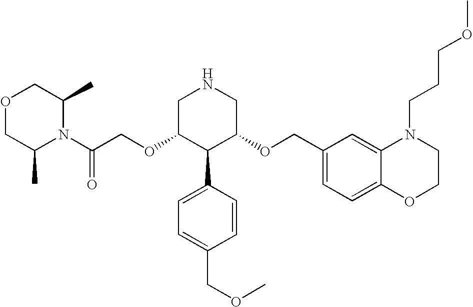 | yellowish oil | 0.27 (A) | 3.62 (I) |
| 73 | 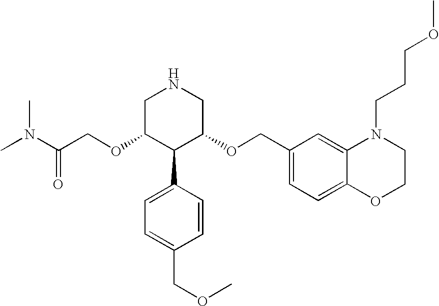 | yellowish oil | 0.27 (A) | 3.50 (I) |
| 74 | 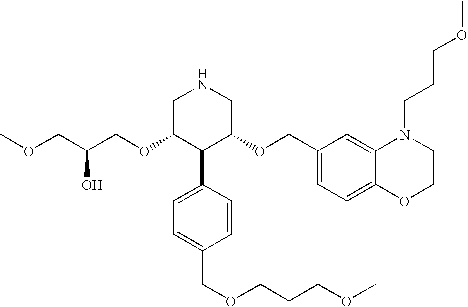 | reddish oil | 0.20 (A) | 3.47 (I) |
| 75 | 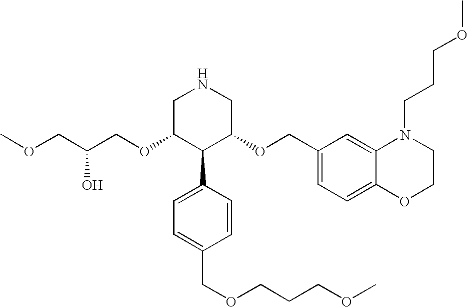 | reddish oil | 0.20 (A) | 3.49 (I) |
| 76 | 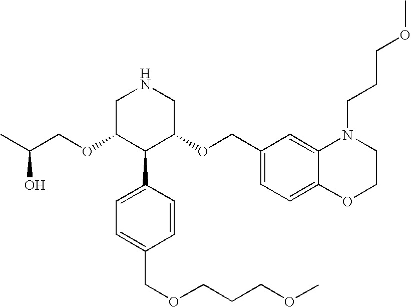 | yellowish oil | 0.28 (A) | 3.74 (I) |
| 77 | 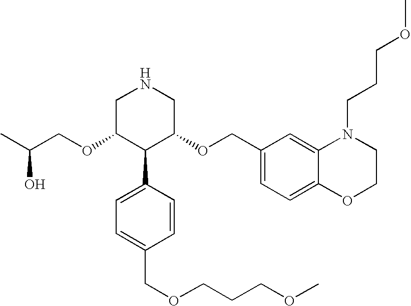 | yellow oil | 0.30 (A) | 3.44 (I) |
| 78 | 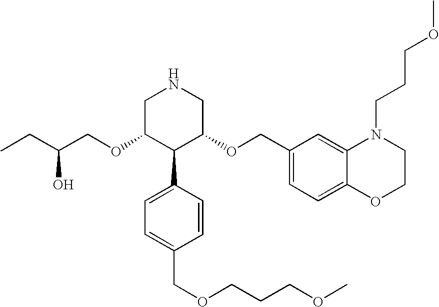 | yellow oil | 0.31 (A) | 3.65 (I) |
| 79 | 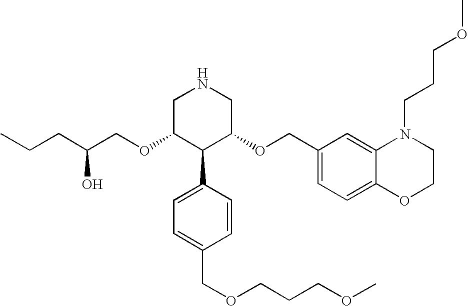 | yellow oil | 0.29 (C) | 3.83 (I) |
| 80 | 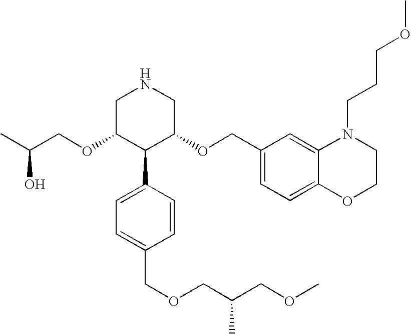 | yellowish oil | 0.29 (A) | 3.82 (I) |
| 81 | 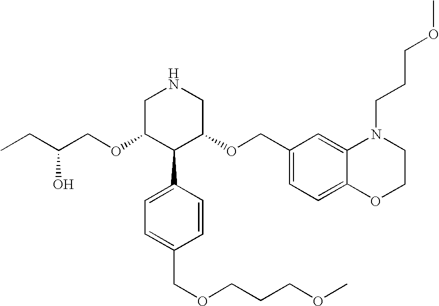 | yellow oil | 0.24 (A) | 3.59 (I) |
| 82 | 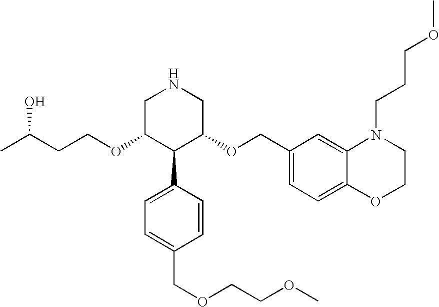 | colourless oil | 0.25 (A) | 3.34 (I) |
| 83 | 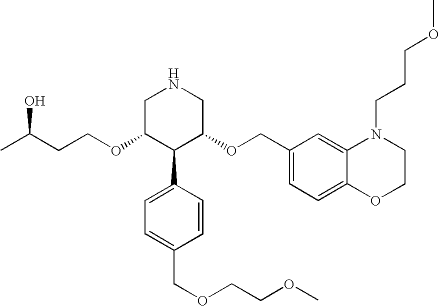 | colourless oil | 0.30 (A) | 3.32 (I) |
| 84 | 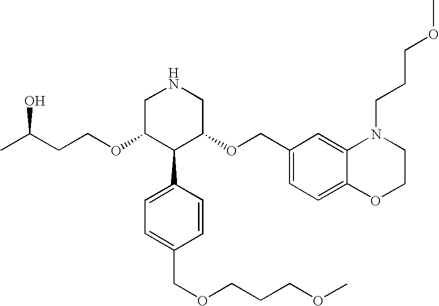 | yellowish resin | 0.32 (G) | 3.54 (I) |
| 85 | 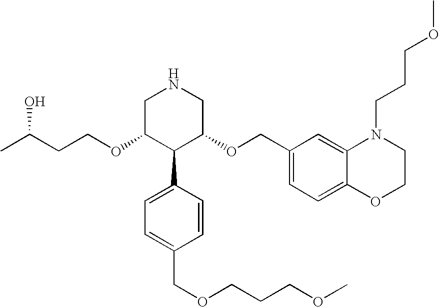 | yellow oil | 0.26 (A) | 3.55 (I) |
| 86 | 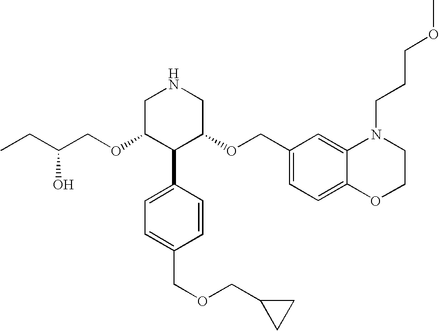 | yellowish oil | 0.30 (A) | 3.98 (I) |
| 87 | 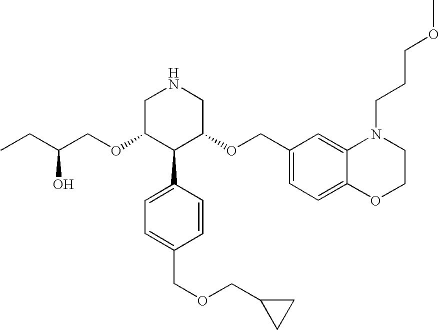 | yellowish oil | 0.31 (A) | 4.01 (I) |
| 88 | 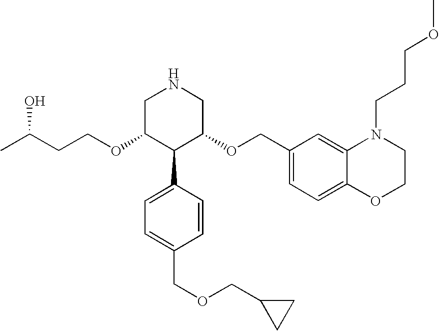 | colourless oil | 0.34 (A) | 3.80 (I) |
| 89 | 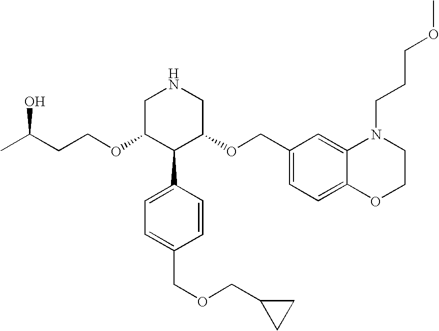 | beige oil | 0.20 (A) | 3.87 (I) |
| 90 | 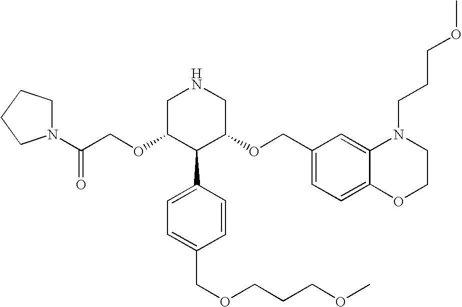 | orange resin | 0.28 (G) | 3.71 (I) |
| 91 | 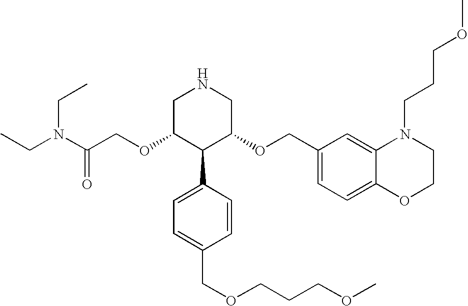 | orange resin | 0.30 (G) | 3.82 (I) |
| 92 | 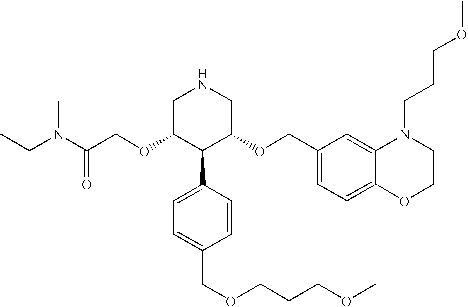 | orange resin | 0.30 (G) | 3.70 (I) |
| 93 | 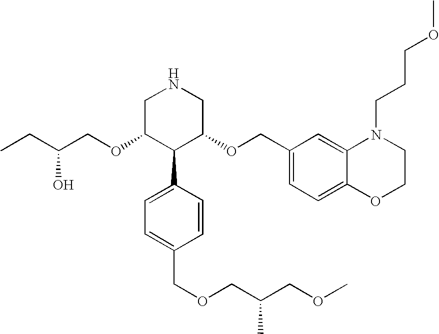 | yellow oil | 0.31 (A) | 3.97 (I) |
| 94 | 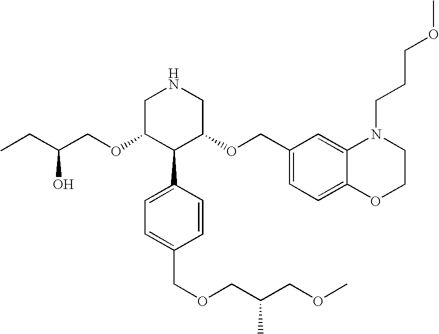 | yellow oil | 0.32 (A) | 4.00 (I) |
| 95 | 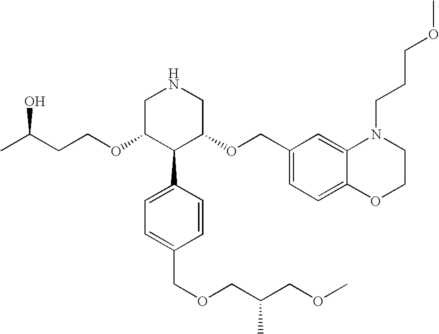 | yellowish oil | 0.20 (C) | 3.85 (I) |
| 96 | 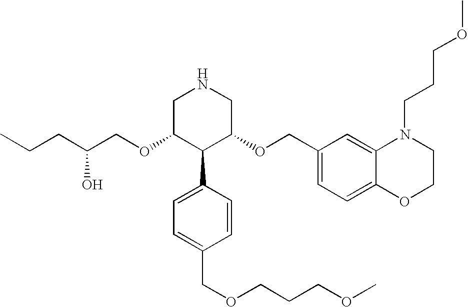 | yellow oil | 0.30 (A) | 3.86 (I) |
| 97 | 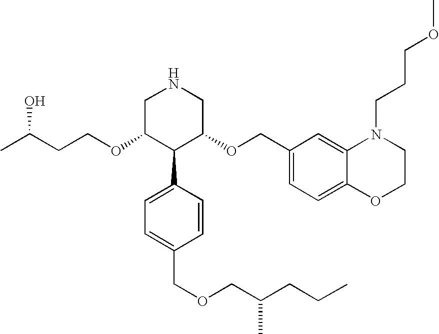 | yellowish oil | 0.22 (A) | 3.86 (I) |
| 98 | 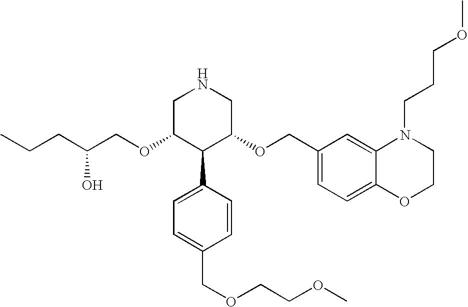 | yellowish oil | 0.08 (A) | 3.64 (I) |
| 99 | 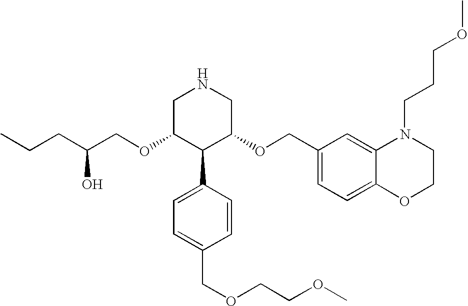 | yellow oil | 0.10 (C) | 3.63 (I) |
| 110 | 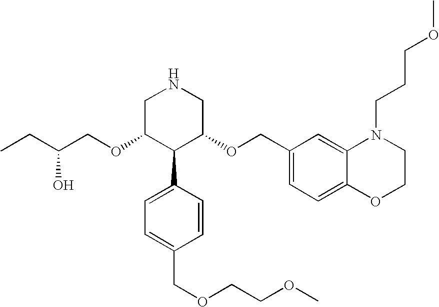 | yellowish resin | 0.14 (A) | 3.43 (I) |
| 111 | 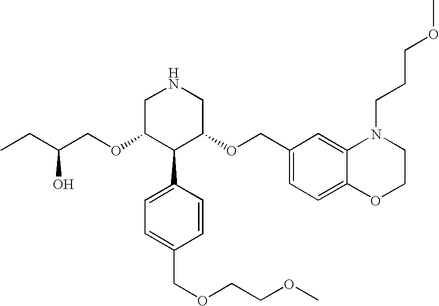 | yellow oil | 0.33 (A) | 3.50 (I) |
| 112 | 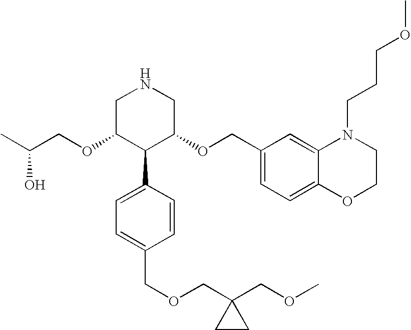 | yellow oil | 0.15 (A) | 3.70 (I) |
| 113 | 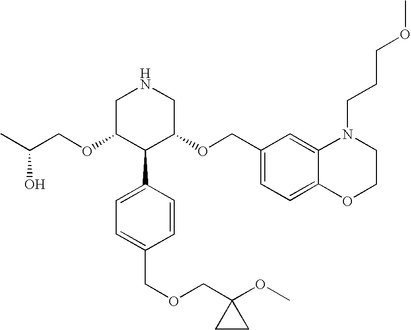 | | | |
| 114 | 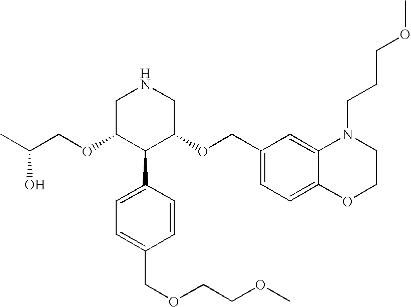 | | | |
| 115 | 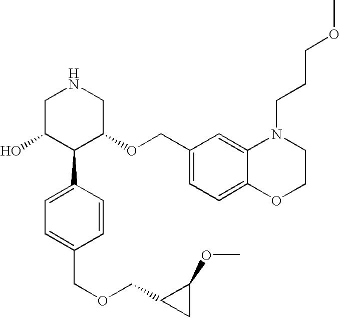 | | | |
| 116 | 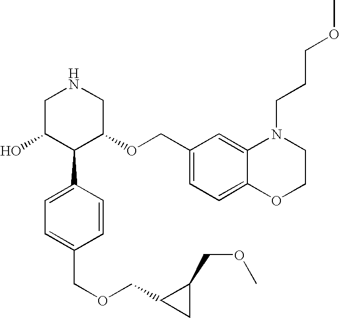 | yellow oil | 0.11 (A) | 3.47 (I) |
| 117 | 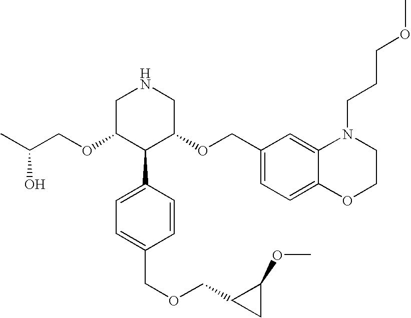 | | | |
| 118 | 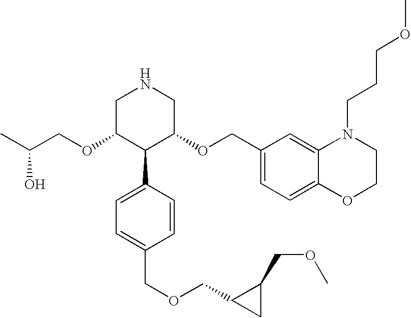 | yellow oil | 0.23 (A) | 3.62 (I) |
| 119 | 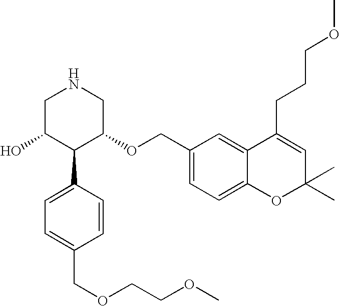 | | | |
| 120 | 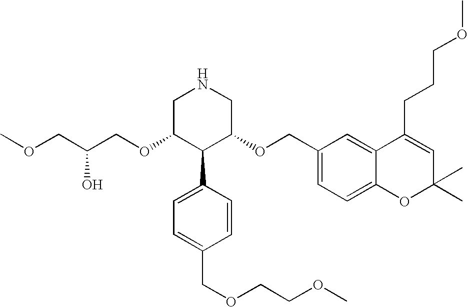 | | | |
| 121 | 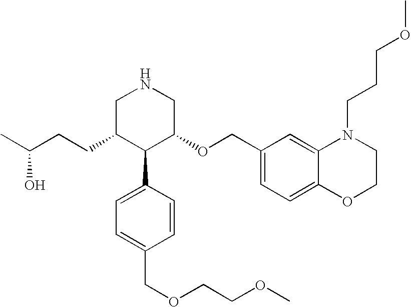 | | | |
| 122 | 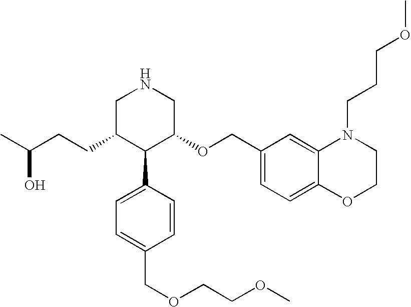 | | | |
| 123 | 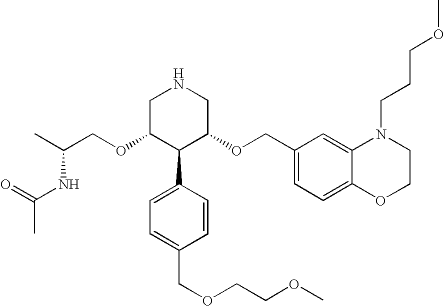 | yellowish oil | 0.16 (A) | 3.33 (I) |
| 124 | 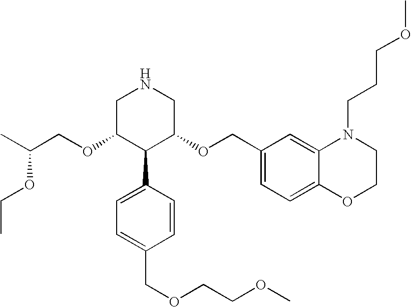 | yellow oil | 0.39 (A) | 3.73 (I) |
| 125 | 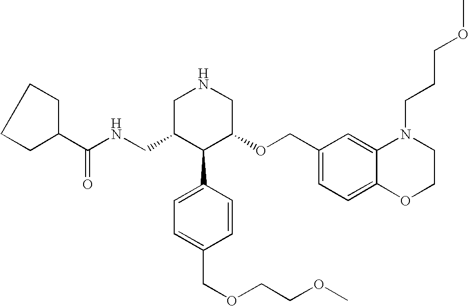 | | | |
| 126 | 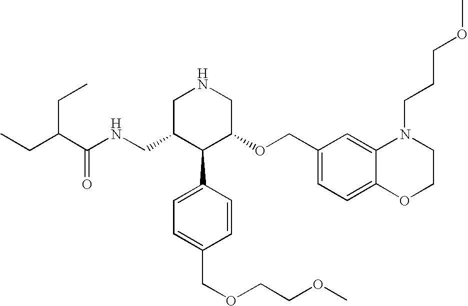 | | | |
| 127 | 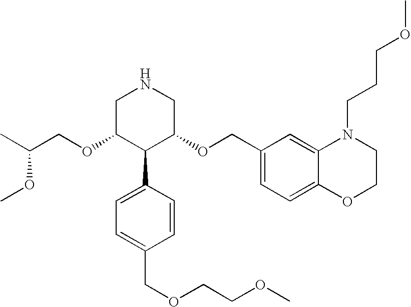 | yellow oil | 0.24 (A) | 3.58 (I) |
| 128 | 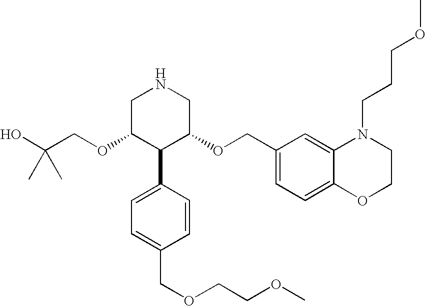 | yellowish oil | 0.31 (C) | 3.44 (I) |
| 129 | 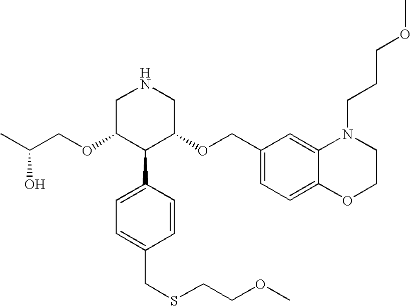 | yellow oil | 0.18 (A) | 3.62 (I) |

Thin-layer chromatography element systems:

A Dichloromethane/methanol/conc. ammonia 25%=200:20:1
B Dichloromethane/methanol/conc. ammonia 25%=200:20:0.5
C Dichloromethane/methanol/conc. ammonia 25%=200:10:1
D Dichloromethane/methanol/conc. ammonia 25%=90:10:1
E Dichloromethane/methanol/conc. ammonia 25%=60:10:1
F Dichloromethane/methanol/conc. ammonia 25%=200:30:1

### G Dichloromethane/methanol=9:1

HPLC gradients on Hypersil BDS C-18 (5 um); column: 4×125 mm
I 90% water*/10% acetonitrile* to 0% water*/100% acetonitrile* in 5 minutes+2.5 minutes (1.5 ml/min)
II 95% water*/5% acetonitrile* to 0% water*/100% acetonitrile* in 40 minutes (0.8 ml/min) * contains 0.1% trifluoroacetic acid

The following abbreviations are used:

M.p. melting point (temperature)
Rf ratio of distance migrated by a substance to the distance of the solvent front from the
starting point in thin-layer chromatography
Rt retention time of a substance in HPLC (in minutes)

### General Method A: (N-BOC Deprotection)

15 ml of methanol and 2.5 ml of 2N HCl are successively added to a solution of 1 mmol “N-BOC derivative” in 5 ml of chloroform, and the mixture is stirred at 60° C. for 18 hours. The reaction mixture is cooled to room temperature, poured into 1M aqueous sodium bicarbonate solution (40 ml) and extracted with tert-butyl methyl ether (2×60 ml). The organic phases are washed with brine (1×60 ml), dried with sodium sulphate and evaporated. The title compound is obtained from the residue by flash chromatography (SiO₂ 60F).

### General Method B: (Hydrogenation)

A solution of 1 mmol of “substrate” in 15 ml of tetrahydrofuran/methanol 1:1 is hydrogenated in the presence of 100-200 mg of Pd/C 10% at 15-20° C. for 2-20 hours. The reaction mixture is clarified by filtration and the filtrate is evaporated. The title compound is obtained from the residue by flash chromatography (SiO₂ 60F).

### General Method C: (9-BBN Reduction)

A solution of 1 mmol of “lactam” in 3 ml of tetrahydrofuran is mixed with 3.2-6.4 mmol of 9-BBN (0.5M in tetrahydrofuran) and stirred under reflux for 1-2 hours (conversion checked by HPLC). The reaction mixture is cooled to room temperature and, after addition of 3.2-6.4 mmol of ethanolamine, evaporated. The residue is stirred in ethyl acetate/heptane 1:1 (30 ml) at 0° C. overnight and clarified by filtration, and the filtrate is evaporated. The title compound is obtained from the residue by flash chromatography (SiO₂ 60F).

### General Method D: (O-Alkylation)

1.1 mmol of sodium hydride (60% dispersion in oil) are added to a solution of 1 mmol of “alcohol”, 1.0-2.0 mmol of “benzyl halide” in 2.0 ml of N,N-dimethylformamide while stirring at −10° C. The reaction mixture is stirred at −10° C. for 1 hour and at room temperature for 18 hours. The mixture is poured into 1M aqueous sodium bicarbonate solution (50 ml) and extracted with tert-butyl methyl ether (2×50 ml). The organic phases are washed successively with water (1×50 ml) and brine (1×60 ml), dried with sodium sulphate and evaporated. The title compound is obtained from the residue by flash chromatography (SiO₂ 60F).

### General Method E: (Chlorination)

A solution of 40 mmol of “benzyl alcohol” in 6.40 ml of pyridine and 100 ml of dichloromethane is slowly added dropwise to a solution, precooled to 0-5° C., of 7.65 ml of thionyl chloride in 20 ml of dichloromethane. The reaction mixture is stirred at 0° C. and then at room temperature for 1 hour each, and then poured into 200 ml of ice-water. The mixture is extracted with dichloromethane (2×200 ml). The organic phases are washed successively with 1M aqueous sodium bicarbonate solution (2×200 ml) and brine, dried with sodium sulphate and evaporated. The title compound is obtained from the residue by flash chromatography (SiO₂ 60F).

### General Method F: (Phenol Alkylation I)

A mixture of 20 mmol of “phenol” in 60 ml of N,N-dimethylformamide with 4.15 g of potassium carbonate and 30 mmol of “halide” or “tosylate” is stirred at 100° C. for 24 hours. The reaction mixture is then evaporated. The residue is mixed with 1M aqueous sodium bicarbonate solution (40 ml) and extracted with ethyl acetate (2×60 ml). The organic phases are washed with brine (1×60 ml), dried with sodium sulphate and evaporated. The title compound is obtained from the residue by flash chromatography (SiO₂ 60F).

### General Method G: (Phenol Alkylation II)

A suspension of 1 mmol of “tosylate”, 2 mmol “phenol”, 2 mmol of potassium carbonate and 20 ml of acetonitrile is stirred at 90° C. for 24 h. The reaction mixture is then evaporated. The residue is mixed with saturated aqueous sodium bicarbonate solution and extracted with ethyl acetate (2×). The organic phases are washed with brine, dried with sodium sulphate and evaporated. The title compound is obtained from the residue by flash chromatography (SiO₂ 60F).

### General Method H: (Tosylation)

A solution of 12 mmol of p-toluenesulphonyl chloride in 15 ml of dichloromethane is added dropwise to a solution of 10 mmol of “alcohol”, 15 mmol of triethylamine, 1 mmol of 4-dimethylaminopyridine in 90 ml of dichloromethane at 0° C. The reaction mixture is stirred at room temperature for 2-18 hours. The reaction mixture is diluted with dichloromethane and then washed with water and brine, dried with sodium sulphate and evaporated. The title compound is obtained from the residue by flash chromatography (SiO₂ 60F).

### General Method I: (Phenol Alkylation III)

A suspension of 1 mmol of “phenol”, 1.0-1.5 mmol of “tosylate” or “bromide”, 1.5 mmol of caesium carbonate and 2 ml of acetonitrile is stirred at 80° C. for 2 hours. The reaction mixture is cooled, poured into water and extracted with ethyl acetate (2×). The organic phases are washed with brine, dried with sodium sulphate and evaporated. The title compound is obtained from the residue by flash chromatography (SiO₂ 60F).

### General Method J (Alcohol Desilylation)

A solution of 1 mmol of “silyl ether” in 5 ml of tetrahydrofuran is mixed with 1.5-2.0 mmol of tetrabutylammonium fluoride (1M solution in tetrahydrofuran), and the solution is stirred at room temperature for 1-2 hours. The reaction solution is then diluted with water and extracted 2× with tert-butyl methyl ether. The combined organic phases are dried with sodium sulphate and evaporated. The title compound is obtained from the residue by flash chromatography (SiO₂ 60F).

### General Method K (Borane Reduction)

A solution of 1 mmol of “lactam” in 3 ml of tetrahydrofuran is mixed with 3.0-6.0 mmol of borane-tetrahydrofuran complex (1M in tetrahydrofuran) and stirred at room temperature for 1-3 hours (conversion checked by HPLC or TLC). The reaction mixture is mixed with 3.0-6.0 mmol of methanol and evaporated. The title compound is obtained from the residue by flash chromatography (SiO₂ 60F).

### General Method L (N-Tos Deprotection 1)

0.44 mmol of sodium dihydrogen phosphate and 0.90 mmol of sodium amalgam (10% Na) are successively added at room temperature to a solution of 0.09 mmol of “tosylamide” in 10 ml of methanol. The reaction mixture is stirred for 2-18 hours, diluted with water and extracted with ethyl acetate. The organic phase is separated off and washed with brine, dried with sodium sulphate and evaporated. The title compound is obtained from the residue by flash chromatography (SiO₂ 60F).

### General Method M (O-Alkylation II)

1 mmol of methylmagnesium bromide (35% solution in diethyl ether) is added to a solution of 1 mmol of “secondary alcohol” in 5 ml of tetrahydrofuran at room temperature. The reaction solution is heated to reflux for 5 minutes and then a solution of 2.2 mmol of “oxirane” in 1 ml of THF is added. The reaction mixture is heated to reflux for 1-5 hours and poured into saturated aqueous sodium bicarbonate solution, and the mixture is extracted with tert-butyl methyl ether. The combined organic phases are dried over sodium sulphate and evaporated. The title compound is obtained from the residue by flash chromatography (SiO₂ 60F).

### General Method N(N-Tos Deprotection II)

0.5 ml of a bluish green sodium naphthalenide stock solution (from 0.04 g of sodium and 0.22 g of naphthalene in 5 ml of dimethoxyethane) is added to a solution of 0.1 mmol of “tosylamide” in 2 ml of dimethoxyethane at −60° C. After 36 hours, the reaction mixture is diluted with water and extracted with dichloromethane (2×). The combined organic phases are washed with brine, dried with sodium sulphate and evaporated. The title compound is obtained from the residue by flash chromatography (SiO₂ 60F).

### Example 4

### 4(S)-[4-(3-Ethoxy-2(S)-methylpropoxymethyl)phenyl]-5(R)[4-(3-methoxypropyl)-3,4-dihydro-2H-benzo[1,4]oxazin-6-ylmethoxy]piperidin-3(S)-ol

The title compound is prepared in analogy to method L from 0.44 g of (3S,4S,5R)-4-[4-((S)-3-ethoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol.

The starting materials are prepared as follows:

### a) (3S,4S,5R)-4-[4-((S)-3-Ethoxy-2-methyloropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

0.62 g of 6-[(3R,4R,5S)-4-[4-((S)-3-ethoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine are reacted in analogy to method J. The title compound is obtained as a yellowish resin. Rf=0.33 (EtOAc/heptane 2:1); Rt=5.35 (gradient I).

### b) 6-[(3R,4R,5S)-4-[4-((S-3-Ethoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.119 g of sodium hydride dispersion (60%) is added to a mixture of 0.92 g of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)3,4-dihydro-2H-benzo[1,4]oxazine and 0.30 g of (R)-3-ethoxy-2-methylpropan-1-ol in 20 ml of N,N-dimethylformamide at 0° C. After stirring for 22 hours, the reaction mixture is mixed with saturated sodium bicarbonate solution and extracted with tert-butyl methyl ether (3×). The combined organic phases are washed with brine, dried with sodium sulphate and evaporated. The title compound is obtained as a colourless oil from the residue by flash chromatography (SiO₂ 60F). Rf=0.31 (EtOAc/heptane 1:2).

### c) 6-[(3R,4R,5S)-4-(4-Chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine

0.633 ml of methanesulphonyl chloride is added to a mixture of 5.28 g of {4-[(3R,4R,5S)-3-[4-(3-methoxypropyl)-3,4-dihydro-2H-benzo[1,4]oxazin-6-ylmethoxy]-1-(toluene-4-sulphonyl)-5-triisopropylsilanyloxypiperidin-4-yl]phenyl}methanol, 1.48 ml of triethylamine and 0.185 g of tetrabutylammonium chloride in 100 ml of dichloromethane at room temperature. After 66 hours, the reaction mixture is diluted with tert-butyl methyl ether and washed successively with water and brine, dried with sodium sulphate and evaporated. The title compound is obtained as a yellowish resin from the residue. Rf=0.26 (EtOAc/heptane 1:2).

### d) {4-[(3R,4R,5S)-3-[4-(3-Methoxypropyl)-3,4-dihydro-2H-benzo[1,4]oxazin-6-ylmethoxy]-1-(toluene-4-sulphonyl)-5-triisopropylsilanyloxypiperidin-4-yl]-phenyl}methanol

1.43 g of toluenesulphonyl chloride are added to a mixture of 4.16 g of (4-{(3R,4R,5S)-3-[4-(3-methoxypropyl)-3,4-dihydro-2H-benzo[1,4]oxazin-6-ylmethoxy]-5-triisopropylsilanyloxypiperidin-4-yl}phenyl)methanol in 125 ml of ethyl acetate and 125 ml of 2N sodium carbonate solution at room temperature. After 14 hours, the phases are separated and the aqueous phase is extracted with ethyl acetate. The combined organic phases are washed with brine, dried with sodium sulphate and evaporated. The title compound is obtained as an orange resin. Rf=0.30 (EtOAc/heptane 1:1); Rt=29.47 (II).

### e) (4-{(3R,4R,5S)-3-[4-(3-Methoxypropyl)-3,4-dihydro-2H-benzo[1,4]oxazin-6-ylmethoxy]-5-triisopropylsilanyloxypiperidin-4-yl}phenyl)methanol

5.0 g of benzyl (3R,4R,5S)-4-(4-hydroxymethylphenyl)-3-[4-(3-methoxypropyl)-3,4-dihydro-2H-benzo[1,4]oxazin-6-ylmethoxy]-5-triisopropylsilanyloxypiperidine-1-carboxylate are reacted in analogy to method B. The title compound is obtained as an orange resin. Rt=4.66 (gradient I).

### f) Benzyl (3R,4R,5S)-4-(4-hydroxymethylphenyl)-3-[4-(3-methoxypropyl)-3,4-dihydro-2H-benzo[1,4]oxazin-6-ylmethoxy]-5-triisopropylsilanyloxypiperidine-1-carboxylate

27.47 g of benzyl (3R,4R,5S)-4-(4-carboxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate are reacted in analogy to method K. The title compound is obtained as an orange resin. Rf=0.14 (EtOAc/heptane 1:2).

### g) Benzyl (3R,4R,5S)-4-(4-carboxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

A mixture of 28.85 g of benzyl (3R,4R,5S)-4-(4-methoxycarbonylphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate in 250 ml of tetrahydrofuran and 144.5 ml 1N NaOH is stirred at 80° C. for 48 hours. The reaction mixture is cooled, mixed with 100 ml of 2N HCl and extracted with tert-butyl methyl ether (3×750 ml). The combined organic phases are evaporated, stripped with toluene, taken up in ethyl acetate, dried with sodium sulphate, filtered and evaporated, and the crude title compound is obtained as a yellowish resin. Rt=6.31 (gradient I).

### h) Benzyl (3R,4R,5S)-4-(4-methoxycarbonylphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

36.80 g of benzyl (3R,4R,5S)-3-hydroxy-4-(4-methoxycarbonylphenyl)-5-triisopropylsilanyloxypiperidine-1-carboxylate and 22.60 g of 6-chloromethyl-4-(3-methoxypropyl)-4H-benzo[1,4]oxazin-3-one are reacted in analogy to method D. The title compound is obtained as a yellowish oil. Rf=0.14 (EtOAc/heptane 1:2); Rt=6.86 (gradient I).

### i) Benzyl (3R,4R,5S-3-hydroxy-4-(4-methoxycarbonylphenyl)-5-triisopropylsilanyloxypiperidine-1-carboxylate

160 ml of N,N-dimethylformamide, 116 ml of methanol, 1.08 g of diphenylphosphinopropane and 0.582 g of palladium(II) acetate are introduced into an autoclave under argon. The reaction mixture is stirred at room temperature for 20 minutes. Then 66.73 g of benzyl (3R,4R,5S)-3-hydroxy-4-(4-trifluoromethanesulphonyloxyphenyl)-5-triisopropylsilanyloxypiperidine-1-carboxylate and 16 ml of triethylamine are added, and the autoclave is loaded with 5 bar of carbon monoxide. The reaction mixture is then stirred under a pressure of 5 bar at 70° C. for 3 hours. The reaction mixture is subsequently cooled, and a solution of palladium(II) acetate (0.293 g) and diphenylphosphinopropane (0.539 g) in 90 ml of DMF and 65 ml of methanol is added. The reaction mixture is then stirred under 5 bar of carbon monoxide at 70° C. for a further 3 hours. The reaction solution is cooled and stirred with 580 ml of water and 180 ml of tert-butyl methyl ether. The phases are separated and the aqueous phase is extracted twice more with 180 ml of tert-butyl methyl ether. The organic phases are combined and evaporated to dryness. The title compound is obtained as an orange oil from the residue by flash chromatography (SiO₂ 60F). Rf=0.19 (EtOAc/heptane 1:2).

### j) Benzyl (3R,4R,5S)-3-hydroxy-4-(4-trifluoromethanesulphonyloxyphenyl)-5-triisopropylsilanyloxypiperidine-1-carboxylate

40.55 g of N-phenyl(trifluoromethanesulphonimide) and then 16.2 ml of triethylamine are added to a solution of 52.95 g of benzyl (3R,4R,5S-3-hydroxy-4-(4-hydroxyphenyl)-5-triisopropylsilanyloxypiperidine-1-carboxylate at room temperature. After 3.5 hours, the reaction mixture is mixed with 150 g of SiO₂ and evaporated. The title compound is obtained as a yellow oil from the residue by flash chromatography (SiO₂ 60F). Rf=0.30 (EtOAc/heptane 1:2); Rt=6.51 (gradient I).

### k) Benzyl (3R,4R,5S)-3-hydroxy-4-(4-hydroxyphenyl)-5-triisopropylsilanyloxypiperidine-1-carboxylate

A solution of 63.0 g of (3R,4R,5S)-4-(4-hydroxyphenyl)-5-triisopropylsilanyloxypiperidin-3-ol in 800 ml of ethyl acetate is mixed with 800 ml of saturated sodium bicarbonate solution. The two-phase mixture is cooled to 0° C. and, after slow addition of 31.1 g of benzyl chloroformate, stirred for 2 hours. The reaction mixture is extracted with ethyl acetate/tetrahydrofuran. The organic phases are evaporated, and the title compound is obtained as a white foam by crystallization (EtOAc/heptane) from the residue. Rf=0.38 (EtOAc/heptane 1:1); Rt=5.77 (gradient I).

### l) (3R,4R,5S)-4-(4-Hydroxyphenyl)-5-triisopropylsilanyloxypiperidin-3-ol

5.210 g of (3R,4R,5S)-4-(4-benzyloxyphenyl)-1-((R)-1-phenylethyl)-5-triisopropylsilanyloxypiperidin-3-ol are reacted in analogy to method B. The title compound is obtained as a colourless solid. Rf=0.19 (dichloromethane/methanol/25% conc. ammonia=200:20:1); Rt=3.80 (gradient I).

### m) (3R,4R,5S)-4-(4-Benzyloxyphenyl)-1-((R)-1-phenylethyl)-5-triisopropylsilanyloxypiperidin-3-ol

150 ml of borane-tetrahydrofuran complex (1M in tetrahydrofuran) are added dropwise toga solution of 20.00 g of (S)-4-(4-benzyloxyphenyl)-1-((R)-1-phenylethyl)-3-triisopropyl-silanyloxy-1,2,3,4-tetrahydropyridine in 280 ml of 1,2-dimethoxyethane at 0° C. The reaction solution is then stirred at 30° C. for 3 hours. The solution is cooled to room temperature and hydrolysed with 70 ml of water. The hydrolysed solution is stirred for 5 minutes and then 56.00 g of sodium percarbonate are added, and the suspension is stirred at 50° C. for 1 hour. The reaction mixture is poured into 600 ml of water and extracted with ethyl acetate (2×). The combined organic phases are washed with 400 ml each of water and brine and evaporated. The title compound is obtained as a yellowish oil from the residue by flash chromatography (SiO₂ F60). Rf=0.23 (EtOAc/heptane 1:2); Rt=5.75 (gradient I).

### n) (S)-4-(4-Benzyloxyphenyl)-1-((R)-1-phenylethyl)-3-triisopropylsilanyloxy-1,2,3,6-tetrahydropyridine

A suspension of 14.70 g of 4-(4-benzyloxyphenyl)-1-(1(R)phenylethyl)-1,2,3,6-tetrahydropyridin-3(S)-ol [257928-45-3] in 250 ml of dichloromethane is mixed with 6.80 ml of 2,6-lutidine and cooled to 0° C. 12.60 ml of triisopropylsilyl trifluoromethanesulphonate are added dropwise, and the reaction mixture is stirred at 0° C. for 1 hour. The reaction solution is poured into 400 ml of water, and the phases are separated. The aqueous phase is back-extracted with 200 ml of dichloromethane, and the combined organic phases are dried with sodium sulphate and evaporated. The title compound is obtained as a yellowish brown oil from the residue by flash chromatography (SiO₂ F60). Rf=0.66 (EtOAc/heptane 1:2); Rt=5.83 (gradient I).

### o) 6-Chloromethyl-4-3-methoxypropyl)-4H-benzo[1,4]oxazin-3-one

0.37 g of 6-hydroxymethyl-4-(3-methoxypropyl)-4H-benzo[1,4]oxazin-3-one is reacted in analogy to method E. The title compound is obtained as a colourless oil. Rf=0.60 (EtOAc/heptane 2:1); Rt=4.05 (gradient I).

### p) 6-Hydroxymethyl-4-(3-methoxypropyl)-4H-benzo[1,4]oxazin-3-one

A suspension of 1.79 g of 6-hydroxymethyl-4H-benzo[1,4]oxazin-3-one, 2.20 ml of 1-chloro-3-methoxypropane, 10 g of potassium fluoride on alumina and 0.033 g of potassium iodide in 150 ml of acetonitrile is stirred under reflux for 72 hours. The reaction mixture is cooled and clarified by filtration, and the filtrate is evaporated to dryness. The title compound is obtained as a yellow oil from the residue by flash chromatography (SiO₂ 60F). Rf=0.60 (dichloromethane/methanol 9:1); Rt=2.74 (gradient I).

### q) 6-Hydroxymethyl-4H-benzo[1,4]oxazin-3-one

A mixture of 6.9 g of methyl 3-oxo-3,4-dihydro-2H-benzo[1,4]oxazine-6-carboxylate [202195-67-3] and 230 ml of tetrahydrofuran is cooled to −40° C. 88.9 ml of diisobutylaluminium hydride (1.5M in toluene) are added dropwise over the course of 30 minutes at −40° C. The reaction mixture is stirred at −40° C. to −20° C. for 1.5 hours and then cautiously poured into 150 ml of 2N HCl (cold). The organic phase is separated off and the aqueous phase is extracted with tetrahydrofuran (5×100 ml). The organic phases are washed with brine (1×100 ml), filtered through cotton wool and evaporated. The title compound is obtained as beige crystals from the residue by crystallization (from ethanol). Rf=0.16 (EtOAc/heptane 2:1); Rt=2.23 (gradient I); m.p.: 186-187° C.

The following compounds are prepared in an analogous manner to the process described in Example 4:

### Examples

### 8 5(R)-[4-(3-Methoxypropyl)-3,4-dihydro-2H-benzo[1,4]oxazin-6-ylmethoxy]-4(S)-[4-(tetrahydropyran-4-ylmethoxymethyl)phenyl]piperidin-3(S)-ol

### 42 (3S,4S,5R)-4-(4-Cyclopropylmethoxymethylphenyl)-5-[4-(3-methoxypropyl)-3,4-dihydro-2H-benzo[1,4]oxazin-6-ylmethoxy]piperidin-3-ol

### Example 5

### (3S,4S,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-ol

The title compound is prepared in analogy to method L from 0.35 g of (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol.

The starting materials are prepared as follows:

### a) (3S,4S,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

1.42 g of 6-[(3R,4R,5S)-4-[4-((S)-3-methoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine are reacted in analogy to method J. The title compound is obtained as a yellowish resin. Rf=0.24 (EtOAc/heptane 2:1); Rt=5.13 (gradient I).

### b) 6-[(3R,4R,5S)-4-[4-((S)-3-Methoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

2 g of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and 0.551 g of (R)-3-methoxy-2-methylpropan-1-ol are reacted in analogy to Example 4b. The title compound is obtained as a yellowish resin. Rf=0.26 (EtOAc/heptane 1:2).

### c) (R-3-Methoxy-2-methylpropan-1-ol

3.03 g of triisopropyl-(3-methoxy-2(S)methylpropoxy)silane are reacted in analogy to method J. The title compound is obtained as a yellowish liquid. Rf=0.15 (EtOAc/heptane 1:4).

### d) Triisopropyl-((S)-3-methoxy-2-methylpropoxy)silane

3.09 g of sodium hydride (60% dispersion in oil) are added to a solution of 9.55 g of (S)-2-methyl-3-triisopropylsilanyloxypropan-1-ol [256643-28-4] and 7.3 ml of methyl iodide in 70 ml of N,N-dimethylformamide at 0° C. After 60 hours at room temperature, the reaction mixture is diluted with tert-butyl methyl ether and washed successively with water and brine, dried with sodium sulphate and evaporated. The title compound is obtained as a yellow oil from the residue by flash chromatography (SiO₂ 60F). Rf=0.41 (EtOAc/heptane 1:10).

### Example 7

### (2-{(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}ethyl)methylamine

The title compound is prepared from 0.28 g of N-{2-[(3S,4R,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]ethyl}-N-methylbenzenesulphonamide in analogy to method L.

The starting material is prepared as follows:

### a) N-{2-[(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-yloxy]ethyl}N-methylbenzenesulphonamide

0.104 g of sodium hydride dispersion (60%) is added to a solution of 0.38 g of (3S,4S,5R)-4-[4-((S)3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol and 0.80 g of 2-[methyl(toluene-4-sulphonyl)amino]ethyl toluene-4-sulphonate in 6 ml of tetrahydrofuran at room temperature, and the mixture is then heated to 45° C. 2-[methyl(toluene-4-sulphonyl)-amino]ethyl toluene-4-sulphonate and sodium hydride dispersion (60%) are again added after 1 and 2 hours. After 3 hours, the reaction mixture is diluted with tert-butyl methyl ether and washed successively with 1:1 water/saturated aqueous sodium bicarbonate solution and brine, dried with sodium sulphate and evaporated. The title compound is obtained as a colourless oil from the residue by flash chromatography (SiO₂ 60F). Rf=0.36 (EtOAc/heptane 2:1); Rt=5.96 (gradient I).

### b) (3S,4S,5R)-4-[4-((S)-3-Methoxy-2-methyloropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

0.62 g of 6-[(3R,4R,5S)-4-[4-((S)-3-methoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine are reacted in analogy to method J. The title compound is obtained as a yellowish resin. Rf=0.24 (EtOAc/heptane 2:1); Rt=5.13 (gradient I).

### c) 6-[(3R,4R,5S)-4-[4-((S)-3-Methoxy-2-methylpropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.92 g of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and 0.30 g of (R)-3-methoxy-2-methylpropan-1-ol (Example 5c) are reacted in analogy to Example 4b. The title compound is obtained as an orange oil. Rf=0.26 (EtOAc/heptane 1:2).

### Example 9

### (3S,4S,5R)-5-[4-(3-Methoxypropyl)-3,4-dihydro-2H-benzo[1,4]oxazin-6-methoxy]-4-[4-((R)-2-methyl-3-methylsulphanylpropoxymethyl)phenyl]piperidin-3-ol

The title compound is obtained from (3S,4S,5R)-5-[4-(3-methoxypropyl)-3,4-dihydro-2H-benzo[1,4]oxazin-6-ylmethoxy]-4-[4-((R)-2-methyl-3-methylsulphanylpropoxymethyl)phenyl]-1-(toluene-4-sulphonyl)piperidin-3-ol in analogy to method N.

The starting materials are prepared as follows:

### a) (3S,4S,5R)-5-[4-(3-Methoxypropyl)-3,4-dihydro-2H-benzo[1,4]oxazin-6-ylmethoxy]-4-[4-((R)-2-methyl-3-methylsulphanylpropoxymethyl)phenyl]-1-(toluene-4-sulphonyl)piperidin-3-ol

6-[(3R,4R,5S)-4-(4-Chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and (R-2-methyl-3-methylsulphanylpropan-1-ol are reacted in analogy to Example 4b. The title compound is obtained as a yellow resin. Rf=0.26 (EtOAc/heptane 1:1); Rt=5.45 (gradient I).

### b) (R-2-Methyl-3-methylsulphanylpropan-1-ol

2.55 g of triisopropyl-((R-2-methyl-3-methylsulphanylpropoxy)silane are reacted in analogy to method J. The title compound is obtained as a yellowish oil. Rf=0.14 (EtOAc/heptane 1:4).

### c) Triisopropyl-((R)-2-methyl-3-methylsulphanylpropoxy)silane

A solution of 2.06 g of sodium methanethiolate in 15 ml of ethanol is added dropwise to a solution of 6.05 g of ((R)-3-bromo-2-methylpropoxy)triisopropylsilane in 60 ml of tetrahydrofuran at room temperature. After 19 hours, the reaction mixture is diluted with diethyl ether and washed successively with water and brine, dried with sodium sulphate and evaporated. The crude title compound is obtained as yellowish oil from the residue. Rf=0.18 (heptane).

### d) ((R)-3-Bromo-2-methylpropoxy)triisopropylsilane

1.51 g of imidazole and 4.26 ml of chlorotriisopropylsilane are added to a solution of 3.15 g of (R)-3-bromo-2-methylpropan-1-ol [93381-28-3] in 50 ml of dichloromethane at 0° C. After 18 hours at room temperature, the reaction mixture is quenched with 200 ml of 0.1N HCl and extracted with diethyl ether (2×)—the combined organic phases are washed with water and brine, dried with sodium sulphate and evaporated. The crude title compound is obtained as a colourless liquid from the residue. Rf=0.75 (EtOAc/heptane 1:10).

### Example 10

### (3S,4S,5R)-4-[4-((2R,3S-3-Methoxy-2-methylbutoxymethyl)phenyl]-5-[4-(3-methoxypropyl)-3,4-dihydro-2H-benzo[1,4]oxazin-6-ylmethoxy]piperidin-3-ol

The title compound is prepared from 0.18 g of (3S,4S,5R)-4-[4-((2R,3S)-3-methoxy-2-methylbutoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol in analogy to method L.

The starting materials are prepared as follows:

### a) (3S,4S,5R)-4-[4-((2R,3S)-3-Methoxy-2-methylbutoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ol

0.282 g of 6-[(3R,4R,5S)-4-[4-((2R,3S)-3-methoxy-2-methylbutoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine is reacted in analogy to method J. The title compound is obtained as a colourless oil. Rf=0.23 (EtOAc/heptane 2:1); Rt=5.25 (gradient I).

### b) 6-[(3R,4R,5S)-4-[4-((2R,3S-3-Methoxy-2-methylbutoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.027 g of sodium hydride (60% dispersion in oil) is added to a solution of 0.278 g of (2S,3R)-4-{4-[(3R,4R,5S)-3-[4-(3-methoxypropyl)-3,4-dihydro-2H-benzo[1,4]oxazin-6-ylmethoxy]-1-(toluene-4-sulphonyl)-5-triisopropylsilanyloxypiperidin-4-yl]-benzyloxy}-3-methylbutan-2-ol and 0.237 g of methyl iodide in 5 ml of tetrahydrofuran at 0° C. After 6 hours at room temperature, the reaction mixture is diluted with 230 ml of tert-butyl methyl ether and washed successively with 70 ml of saturated aqueous sodium bicarbonate solution, 30 ml of water and 50 ml of brine, dried with sodium sulphate and evaporated. The title compound is obtained as a yellowish oil from the residue by flash chromatography (SiO₂ 60F). Rf=0.54 (EtOAc/heptane 1:1).

### c) (2S,3R)-4-{4-[(3R,4R,5S-3-[4-(3-Methoxypropyl)-3,4-dihydro-2H-benzo[1,4]oxazin-6-ylmethoxy]-1-(toluene-4-sulphonyl)-5-triisopropylsilanyloxypiperidin-4-yl]benzyloxy}-3-methylbutan-2-ol

1.04 g of magnesium bromide diethyl etherate complex are added to a solution of 1.46 g of 4-(3-methoxypropyl)-6-[(3R,4R,5S)-4-{4-[(2R,3S-2-methyl-3-(tetrahydropyran-2-yloxy)-butoxymethyl]phenyl}-1-(toluene-4-sulphonyl)-5-triisopropylsilanyloxypiperidin-3-yloxymethyl]-3,4-dihydro-2H-benzo[1,4]oxazine in 24 ml of diethyl ether. After 2 hours, a further 0.5 g of magnesium complex is added. The reaction mixture is stirred vigorously at room temperature for 20 hours and is then quenched at 0° C. successively with 20 ml of saturated aqueous sodium bicarbonate solution and 50 ml of water. The mixture is extracted with 300 ml of ethyl acetate. The organic phase is washed successively with 40 ml of water and 40 ml of brine, dried with sodium sulphate and evaporated. The title compound is obtained as a colourless oil from the residue by flash chromatography (SiO₂ 60F). Rf=0.22 (EtOAc(heptane 1:1).

### d) 4-(3-Methoxypropyl)-6-[(3R,4R,5S)-4-{4-[(2R,3S-2-methyl-3-(tetrahydropyran-2-yloxy)-butoxymethyl]phenyl}1-(toluene-4-sulphonyl)-5-triisopropylsilanyloxypiperidin-3-yloxymethyl]-3,4-dihydro-2H-benzo[1,4]oxazine

1.50 g of 6-[(3R,4R,5S)-4-(4-chloromethylphenyl)-(toluene-4-sulphonyl)-5-triisopropylsilanyloxypiperidin-3-yloxymethyl]-4-(3-methoxypropyl-3,4-dihydro-2H-benzo[1,4]oxazine (Example 4c) and 0.549 g of (2R,3S)-2-methyl-3-(tetrahydropyran-2-yloxy)butan-1-ol are reacted in analogy to Example 4b. The title compound is obtained as a colourless oil. Rf=0.21 (EtOAc/heptane 1:2).

### e) (2R,3S-2-Methyl-3-(tetrahydropyran-2-yloxy)butan-1-ol

A solution of 2.29 g of methyl (2S,3S-2-methyl-3-(tetrahydropyran-2-yloxy)butanecarboxylate in 15 ml of diethyl ether is added dropwise to a suspension of 0.804 g of lithium aluminium hydride in 20 ml of diethyl ether. The reaction solution is then stirred at 0° C. for 2 hours. The solution is hydrolysed at 0° C. successively with 1.3 ml of water and with 1.3 ml of 1N sodium hydroxide solution. The hydrolysed solution is stirred at 0° C. for 1 hour and then filtered through Celite and concentrated. The title compound is obtained as a colourless oil from the residue by flash chromatography (SiO₂ F60). Rf=0.20 und 0.11 (diastereomers of the protective group) (EtOAc/heptane 1:2).

### f) Methyl (2S,3S)-2-methyl-3-(tetrahydropyran-2-yloxy)butanecarboxylate

4.455 g of 3, 4-2H-dihydropyran and 0.027 g of pyridinium p-toluenesulphonate are successively added to a solution of 1.40 g of methyl (2S,3S)-3-hydroxy-2-methylbutanecarboxylate [66767-60-0] in 50 ml of dichloromethane. After 15 hours, the reaction mixture is concentrated. The residue is taken up in 50 ml of diethyl ether, and the white precipitate is filtered off. The filtrate is evaporated and the crude title compound is obtained as a colourless oil. Rf=0.50 (EtOAc/heptane 1:2).

The following compound is prepared in an analogous manner to the process described in Example 10:

### Example

### 11 (3S,4S,5R)-4-[4-((2R,3S-3-Ethoxy-2-methylbutoxymethyl)phenyl]-5-[4-(3-methoxypropyl)-3,4-dihydro-2H-benzo[1,4]oxazin-6-ylmethoxy]piperidin-3-ol

### Example 12

### 6-{(3R,4S,5S)-4-[4-((S)-3-Methoxy-2-methylpropoxymethyl)phenyl]-5-[2-(1H-tetrazol-5-yl)ethoxy]piperidin-3-yloxymethyl}-4-(3-methoxypropyl)-3,4-dihydro-2H-benzo[1,4]oxazine

The title compound is prepared from 0.286 g of 6-[(3R,4S,5S)-4-[4-((S)-3-methoxy-2-methylpropoxymethyl)phenyl]-5-[2-(1H-tetrazol-5-yl)ethoxy]-1-(toluene-4-sulphonyl)piperidin-3-yloxymethyl]-4-(3-methoxypropyl)-3,4-dihydro-2H-benzo[1,4]oxazine in analogy to method L.

The starting materials are prepared as follows:

### a) 6-[(3R,4S,5S)-4-[4-((S)-3-Methoxy-2-methylpropoxymethyl)phenyl]-5-[2-(1H-tetrazol-5-yl)ethoxy]-1-(toluene-4-sulphonyl)piperidin-3-yloxymethyl]-4-(3-methoxypropyl)-3,4-dihydro-2H-benzo[1,4]oxazine

0.033 g of dibutyltin oxide is added to a solution of 0.345 g of 3-[(3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propionitrile and 1.76 g of trimethylsilyl azide in 7 ml of toluene. After 14 hours at 100° C., the reaction solution is quenched with 10 ml of 1N HCl at room temperature. The mixture is extracted three times with 70 ml of ethyl acetate. The combined organic phases are washed with brine, dried with sodium sulphate and evaporated. The title compound is obtained as a brown oil from the residue by flash chromatography (SiO₂ 60F). Rf=0.29 (EtOAc/methanol 10:1); Rt=4.93 (gradient I).

### b) 3-[(3S,4S,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-yloxy]propionitrile

0.365 g of acrylonitrile is added to a solution of 0.47 g of (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 5a) and 0.105 g of 2,3,4,6,7,8,9,10-octahydropyrimido[1,2-a]azepine (DBU) in 3.5 ml of acetonitrile. After 18 hours at 50° C., 0.105 g of DBU and 0.365 g of acrylonitrile are again added to the reaction solution. After 62 hours, the reaction mixture is evaporated. The title compound is obtained as a colourless oil from the residue by flash chromatography (SiO₂ 60F). Rf=0.22 (EtOAc/heptane 1:1); Rt=5.43 (gradient I).

### Example 13

### (S)-1-Methoxy-3-{(3S,4R,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.28 g of (S)-1-methoxy-3-[(3S,4R,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting material is prepared as follows:

### a) (S)-1-Methoxy-3-[(3S,4R,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol

0.335 g of (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 5a) and 0.097 g of R-(−)-glycidyl methyl ether [64491-70-9] are reacted in analogy to method M. The title compound is obtained as a cloudy oil. Rf=0.28 (EtOAc/heptane 2:1); Rt=5.25 (gradient I).

The following compound is prepared in an analogous manner to the process described in Example 13:

### Example

### 14 (R)-1-Methoxy-3-{(3S,4R,5R)-4-[4-((S)-3-methoxy-2-methylpropoxymethyl)Phenyl]-5-[4-(3-methoxypropyl)-3,4-dihydro-2H-benzo[1,4]oxazin-6-ylmethoxy]piperidin-3-yloxy}propan-2-ol

### Example 15

### (R)-1-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-3-methoxypropan-2-ol

The title compound is prepared from 0.455 g of (R)-1-[(3S,4R,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]-3-methoxypropan-2-ol in analogy to method L.

The starting materials are prepared as follows:

### a) (R)-1-[(3S,4R,5R)-4-(4-Cylopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]-3-methoxypropan-2-ol

1.0 g of (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol and 0.290 g of S-(+)-glycidyl methyl ether [64491-68-5] are reacted in analogy to method M. The title compound is obtained as a beige oil. Rf=0.32 (EtOAc/heptane 2:1); Rt=5.25 (gradient I).

### b) (3S,4S,5R)-4-(4-Cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol

5.0 g of 6-[(3R,4R,5S)-4-(4-cyclopropylmethoxymethylphenyl)-(toluene-4-sulphonyl)-5-triisopropylsilanyloxypiperidin-3-yloxymethyl]-4-(3-methoxypropyl)-3,4-dihydro-2H-benzo[1,4]oxazine are reacted in analogy to method J. The title compound is obtained as a white foam. Rf=0.18 (EtOAc/heptane 1:1); Rt=5.14 (gradient I).

### c) 6-[(3R,4R,5S)-4-(4-Cyclopropylmethoxymethylphenyl)-1-(toluene-4-sulphonyl)-5-triisopropylsilanyloxypiperidin-3-yloxymethyl]-4-(3-methoxypropyl)-3,4-dihydro-2H-benzo-[1,4]oxazine

0.544 g of sodium hydride (60% dispersion in oil) is added to a stirred solution of 0.90 g of cyclopropylmethanol in 6 ml of N,N-dimethylformamide at −10° C. After 10 minutes, a solution of 5.30 g of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (example 4c) in 8 ml of tetrahydrofuran is added, and the mixture is then stirred at 0° C. for 3 hours. The reaction mixture is poured into 1M sodium bicarbonate solution (150 ml) and extracted with tert-butyl methyl ether (2×150 ml). The organic phases are washed with water (150 ml) and brine (150 ml), dried with sodium sulphate and evaporated. The title compound is obtained as a yellow oil from the residue by flash chromatography (SiO₂ 60F). Rf=0.46 (EtOAc/heptane 1:1).

The following compound is prepared in an analogous manner to the process described in Example 15:

### Example

### 16 (S)-1-{(3S,4R,5R)-4-(4-Cyclopropylmethoxymethylphenyl)-5-[4-(3-methoxypropyl)-3,4-dihydro-2H-benzo[l 1,4]oxazin-6-ylmethoxy]piperidin-3-yloxy}-3-methoxypropan-2-ol

### Example 17

### (R)-1-{(3S,4R,5R)-4-(4-Ethylphenyl)-5-[4-(3-methoxypropyl)-3,4-dihydro-2H-benzo-[1,4]oxazin-6-Ylmethoxy]piperidin-3-yloxy}-3-methoxypropan-2-ol

The title compound is prepared from 0.11 g of (R)-1-[(3S,4R,5R)-4-(4-ethylphenyl)-5-[4-(3-methoxypropyl)-3,4-dihydro-2H-benzo[1,4]oxazin-6-ylmethoxy]-1-(toluene-4-sulphonyl)-piperidin-3-yloxy]-3-methoxypropan-2-ol in analogy to method L.

The starting materials are prepared as follows:

### a) (R)-1-[(3S,4R,5R)-4-(4-Ethylphenyl)-5-[4-(3-methoxypropyl-3,4-dihydro-2H-benzo-[1,4]oxazin-6-ylmethoxy]-1-(toluene-4-sulphonyl)piperidin-3-yloxy]-3-methoxypropan-2-ol

0.18 g of (3S,4S,5R)-4-(4-ethylphenyl)-5-[4-(3-methoxypropyl)-3,4-dihydro-2H-benzo-[1,4]oxazin-6-ylmethoxy]-1-(toluene-4-sulphonyl)piperidin-3-ol and 0.058 g of S-(+)-glycidyl methyl ether [64491-68-5] are reacted in analogy to method M. The title compound is obtained as a colourless oil. Rf=0.17 (EtOAc/heptane 1:1); Rt=5.30 (gradient I).

### b) (3S,4S,5R)-4-(4-Ethylphenyl)-5-[4-(3-methoxypropyl)-3,4-dihydro-2H-benzo[1,4]oxazin-6-ylmethoxy]-1-(toluene-4-sulphonyl)piperidin-3-ol

0.233 g of 6-[(3R,4R,5S)-4-(4-ethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine is reacted in analogy to method J. The title compound is obtained as a colourless resin. Rf=0.32 (EtOAc/heptane 1:1); Rt=5.20 (gradient I).

### c) 6-[(3R,4R,5S)-4-(4-Ethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine

0.274 g of 6-[(3R,4R,5S)-4-(4-ethylphenyl)-1-(toluene-4-sulphonyl)-5-triisopropylsilanyloxypiperidin-3-yloxymethyl]-4-(3-methoxypropyl)-4H-benzo[1,4]oxazin-3-one is reacted in analogy to method K. The title compound is obtained as a colourless oil. Rf=0.34 (EtOAc/heptane 1:2).

### d) 6-[(3R,4R,5S)-4-(4-Ethylphenyl)-1-(toluene-4-sulphonyl)-5-triisopropylsilanyloxypiperidin-3-yloxymethyl]-4-(3-methoxypropyl)-4H-benzo[1,4]oxazin-3-one

0.483 g of (3R,4R,5S)-4-(4-ethylphenyl)-1-(toluene-4-sulphonyl)-5-triisopropylsilanyloxypiperidin-3-ol and 0.282 g of 6-bromomethyl-4-(3-methoxypropyl)-4H-benzo[1,4]oxazin-3-one are reacted in analogy to method D. The title compound is obtained as a colourless oil. Rf=0.38 (EtOAc/heptane 1:2).

### e) (3R,4R,5S)-4-(4-Ethylphenyl)-1-(toluene-4-sulphonyl)-5-triisopropylsilanyloxypiperidin-3-ol

0.49 g of (3R,4R,5S)-4-(4-ethylphenyl)-5-triisopropylsilanyloxypiperidin-3-ol and 0.223 g of toluenesulphonyl chloride are reacted in analogy to Example 4d. The title compound is obtained as a colourless oil. Rf=0.09 (EtOAc/heptane 1:10); Rt=6.85 (gradient I).

### f) (3R,4R,5S)-4-(4-Ethylphenyl)-5-triisopropylsilanyloxypiperidin-3-ol

0.62 g of benzyl (3R,4R,5S)-3-hydroxy-5-triisopropylsilanyloxy-4-(4-vinylphenyl)piperidine-1-carboxylate in 10 ml of methanol are reacted in analogy to method B. The title compound is obtained as a colourless oil. Rt=5.24 (gradient I).

### g) Benzyl (3R,4R,5S)-3-hydroxy-5-triisopropylsilanyloxy-4-(4-vinylphenyl)piperidine-1-carboxylate

0.639 g of 2-vinyl-4,4,5,5-tetramethyl-1,3,2-dioxaborolane and 0.409 g of potassium carbonate are successively added to a solution of 1.246 g of benzyl (3R,4R,5S)-3-hydroxy-4-(4-trifluoromethanesulphonyloxyphenyl)-5-triisopropylsilanyloxypiperidine-1-carboxylate (Example 4j) in 10 ml of dioxane in a Schlenk tube. The mixture is briefly degassed and 0.184 g of tetrakis-(triphenylphosphine)palladium(0) complex is also added. After 14 hours at 85° C., a further 0.32 g of 2-vinyl-4,4,5,5-tetramethyl-1,3,2-dioxaborolane and 0.09 g of Pd(0) complex are added at room temperature. After 24 hours at 95° C., the reaction mixture is cooled at room temperature, diluted with 200 ml of tert-butyl methyl ether and washed successively with 40 ml of water and 20 ml of brine. The organic phase is dried with sodium sulphate and evaporated. The title compound is obtained as a brown oil from the residue by flash chromatography (SiO₂ 60F). Rf=0.12 (EtOAc/heptane 1:4); Rt=6.54 (gradient I).

### Example 18

### (R)-1-Methoxy-3-[(3S,4R,5R)-5-[4-(3-methoxypropyl)-3,4-dihydro-2H-benzo[1,4]oxazin-6-ylmethoxy]-4-(4-propylphenyl)piperidin-3-yloxy]propan-2-ol

The title compound is prepared from 0.117 g of benzyl (3S,4R,5R)-3-((R)-2-hydroxy-3-methoxypropoxy)-5-[4-(3-methoxypropyl)-3,4-dihydro-2H-benzo[1,4]oxazin-6-ylmethoxy]-4-(4-propylphenyl)piperidine-1-carboxylate in analogy to method B.

The starting materials are prepared as follows:

### a) Benzyl (3S,4R,5R)-3-((R-2-hydroxy-3-methoxypropoxy)-5-[4-(3-methoxypropyl)-3,4-dihydro-2H-benzo[1,4]oxazin-6-ylmethoxy]-4-(4-propylphenyl)piperidine-1-carboxylate

0.299 g of benzyl (3S,4S,5R)-3-hydroxy-5-[4-(3-methoxypropyl)-3,4-dihydro-2H-benzo-[1,4]oxazin-6-ylmethoxy]-4-(4-propylphenyl)piperidine-1-carboxylate and 0.101 g of (S)-(+)-glycidyl methyl ether [64491-68-5] are reacted in analogy to method M. The title compound is obtained as a colourless oil. Rf=0.21 (EtOAc/heptane 1:1); Rt=5.50 (gradient I).

### b) Benzyl (3S,4S,5R)-3-hydroxy-5-[4-(3-methoxypropyl)-3,4-dihydro-2H-benzo[1,4]oxazin-6-ylmethoxy]-4-(4-propylphenyl)piperidine-1-carboxylate

0.423 g of benzyl (3R,4R,5S)-3-[4-(3-methoxypropyl)-3,4-dihydro-2H-benzo[1,4]oxazin-6-ylmethoxy]-4-(4-propylphenyl)-5-triisopropylsilanyloxypiperidine-1-carboxylate is reacted in analogy to method J. The title compound is obtained as a colourless oil. Rf=0.20 (EtOAc/heptane 1:2); Rt=5.38 (gradient I).

### c) Benzyl (3R,4R,5S-3-[4-(3-methoxypropyl)-3,4-dihydro-2H-benzo[1,4]oxazin-6-ylmethoxy]-4-(4-propylphenyl)-5-triisopropylsilanyloxypiperidine-1-carboxylate

1.3 ml of propylmagnesium bromide (2N solution in THF) are added to a solution of 1.75 g of benzyl (3R,4R,5S-3-[4-(3-methoxypropyl)-3,4-dihydro-2H-benzo[1,4]oxazin-6-ylmethoxy]-4-(4-trifluoromethanesulphonyloxyphenyl)-5-triisopropylsilanyloxypiperidine-1-carboxylate, 0.037 g of iron(III) acetylacetonate and 5 ml of N-methylpyrrolidon in 50 ml of tetrahydrofuran in a Schlenk tube. After 1 hour at room temperature, a further 0.037 g of iron(III) complex and 1.3 ml of propylmagnesium bromide are added. After 48 hours, the reaction mixture is diluted with tert-butyl methyl ether and quenched with 1N aqueous HCl. The organic phase is separated and washed successively with water and brine, dried with sodium sulphate and evaporated. The title compound is obtained as a yellowish resin from the residue by flash chromatography (SiO₂ 60F). Rf=0.13 (EtOAc/heptane 1:10).

### d) Benzyl (3R,4R,5S)-3-[4-(3-methoxypropyl)-3,4-dihydro-2H-benzo[1,4]oxazin-6-ylmethoxy]-4-(4-trifluoromethanesulphonyloxyphenyl)-5-triisopropylsilanyloxypiperidine-1-carboxylate

6.53 g of benzyl (3R,4R,5S)-4-(4-hydroxyphenyl)-3-[4-(3-methoxypropyl)-3,4-dihydro-2H-benzo[1,4]oxazin-6-ylmethoxy]-5-triisopropylsilanyloxypiperidine-1-carboxylate and 3.41 g of N-phenyltrifluoromethanesulphonimide in analogy to Example 4j. The title compound is obtained as a reddish oil. Rf=0.61 (EtOAc/heptane 1:1).

### e) Benzyl (3R,4R,5S)-4-(4-hydroxyphenyl)-3-[4-(3-methoxypropyl)-3,4-dihydro-2H-benzo-[1,4]oxazin-6-ylmethoxy]-5-triisopropylsilanyloxypiperidine-1-carboxylate

10.2 g of benzyl (3R,4R,5S)-4-(4-hydroxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate are reacted in analogy to method K. The title compound is obtained as a colourless oil. Rf=0.36 (EtOAc/heptane 1:1).

### f) Benzyl (3R,4R,5S)-4-(4-Hydroxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

1.45 g of tetrakis(tirphenylphosphine)palladium(0) complex are added to a mixture of 12.58 g of benzyl (3R,4R,5S)-4-(4-allyloxyphenyl)-3-[4-(3-methoxypropyl)-oxo-3,4-dihydro-2H-benzo[1,4]oxazin-6-ylmethoxy]-5-triisopropylsilanyloxypiperidine-1-carboxylate and 6.33 g of potassium carbonate in 100 ml of methanol. After 5 hours at room temperature, the reaction mixture is filtered and the filtrate is evaporated. The title compound is obtained as a yellowish resin from the residue by flash chromatography (SiO₂ 60F). Rf=0.25 (EtOAc/heptane 1:1); Rt=6.39 (gradient I).

### g) Benzyl (3R,4R,5S)-4-(4-allyloxyphenyl)-3-[4-(3-methoxypropyl)-3-oxo-3,4-dihydro-2H-benzo[1,4]oxazin-6-ylmethoxy]-5-triisopropylsilanyloxypiperidine-1-carboxylate

10.1 g of benzyl (3R,4R,5S)-4-(4-allyloxyphenyl)-3-hydroxy-5-triisopropylsilanyloxypiperidine-1-carboxylate and 5.55 g of 6-chloromethyl-4-(3-methoxypropyl)-4H-benzo[1,4]oxazin-3-one (Example 4o) are reacted in analogy to method D. The title compound is obtained as a yellowish oil. Rf=0.43 (EtOAc/heptane 1:1); Rt=7.13 (gradient I).

### h) Benzyl (3R,4R,5S)-4-(4-allyloxyphenyl)-3-hydroxy-5-triisopropylsilanyloxypiperidine-1-carboxylate

76.2 g of benzyl (3R,4R,5S-3-hydroxy-4-(4-hydroxyphenyl)-5-triisopropylsilanyloxypiperidine-1-carboxylate (Example 4k) and 30.22 g of allyl bromide are reacted at 60° C. in analogy to method F. The title compound is obtained as a yellowish resin. Rf=0.33 (EtOAc/heptane 1:2); Rt=6.59 (gradient I).

The following compound is prepared in an analogous manner to the process described in Example 18:

### Example

### 19 (R)-1-{(3S,4R,5R)-4-(4-Butylphenyl)-5-[4-(3-methoxypropyl)-3,4-dihydro-2H-benzo[1,4]oxazin-6-ylmethoxy]piperidin-3-yloxy}-3-methoxypropan-2-ol

### Example 20

### (R)-1-{(3S,4R,5R)-4-(4-Ethoxymethylphenyl)-5-[4-(3-methoxypropyl)-3,4-dihydro-2H-benzo[1,4]oxazin-6-ylmethoxy]piperidin-3-yloxy}-3-methoxypropan-2-ol

The title compound is prepared from 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and ethanol in analogy to the process described in Example 13 and Example 5.

### Example 21

### (R)-1-Methoxy-3-{(3S,4R,5R)-4-(4-methoxymethylphenyl)-5-[4-(3-methoxypropyl)-3,4-dihydro-2H-benzo[1,4]oxazin-6-ylmethoxy]piperidin-3-yloxy}propan-2-ol

The title compound is prepared from 0.28 g of (R)-1-methoxy-3-[(3S,4R,5R)-4-(4-methoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting materials are prepared as follows:

### a) (R)-1-Methoxy-3-[(3S,4R,5R)-4-(4-methoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]-propan-2-ol

0.370 g of (3S,4S,5R)-4-(4-methoxymethylphenyl)-5-[4-(3-methoxypropyl)-3,4-dihydro-2H-benzo[1,4]oxazin-6-ylmethoxy]-1-(toluene-4-sulphonyl)piperidin-3-ol and 0.119 g of S-(+)-glycidyl methyl ether [64491-68-5] are reacted in analogy to method M. The title compound is obtained as a colourless resin. Rf=0.06 (EtOAc/heptane 1:1); Rt=4.84 (gradient I).

### b) (3S,4S,5R)-4-(4-Methoxymethylphenyl)-5-[4-(3-methoxypropyl)-3,4-dihydro-2H-benzo[1,4]oxazin-6-ylmethoxy]-1-(toluene-4-sulphonyl)piperidin-3-ol

The title compound is prepared from 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and methanol in analogy to the process described in Example 15. The title compound is obtained as a grey resin. Rf=0.11 (EtOAc/heptane 1:1); Rt=4.74 (gradient I).

### Example 22

### (R)-1-Methoxy-3-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.17 g of (R)-1-methoxy-3-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1 toluene-4-sulphonyl)piperidin-3-yloxy]propan-2-ol in analogy to method L.

The starting materials are prepared as follows:

### a) (R)-1-Methoxy-3-[(3S,4R,5R)-4-4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol

0.260 g of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol and 0.078 g of S-(+)-glycidyl methyl ether [64491-68-5] are reacted in analogy to method M. The title compound is obtained as a colourless resin. Rf=0.23 (EtOAc/heptane 4:1); Rt=4.76 (gradient I).

### b) (3S,4S,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

0.380 g of 6-[(3R,4R,5S)-4-[4-(2-methoxyethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine is reacted in analogy to method J. The title compound is obtained as a yellow oil. Rf=0.35 (EtOAc/heptane 4:1); Rt=4.62 (gradient I).

### c) 6-[(3R,4R,5S)-4-[4-(2-Methoxyethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.026 g of sodium hydride (60% dispersion in oil) is added to a solution of 0.40 g of {4-[(3R,4R,5S)-3-[4-(3-methoxypropyl)-3,4-dihydro-2H-benzo[1,4]oxazin-6-ylmethoxy]-1-(toluene-4-sulphonyl)-5-triisopropylsilanyloxypiperidin-4-yl]phenyl}methanol (Example 4d), 0.11 g of 2-bromoethyl methyl ether and 0.19 g of tetrabutylammonium iodide in 2 ml of N,N-dimethylformamide at −5° C. and stirred at room temperature for 18 hours. The reaction mixture is poured into ice-water and extracted with tert-butyl methyl ether. The organic phases are washed with water and brine, dried with sodium sulphate and evaporated. The title compound is obtained as a yellow oil from the residue by flash chromatography (SiO₂ 60F). Rf=0.4 (EtOAc/heptane 1:1).

The following compounds are prepared in an analogous manner to the process described in Example 22

### Examples

### 74 (S)-1-Methoxy-3-{(3S,4R,5R)-4-[4-(3-methoxypropoxymethyl)phenyl]-5-[4-(3-methoxypropyl)-3,4-dihydro-2H-benzo[1,4]oxazin-6-ylmethoxy]piperidin-3-yloxy}-propan-2-ol

Starting from (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 48a).

### 75 (R)-1-Methoxy-3-{(3S,4R,5R)-4-[4-(3-methoxypropoxymethyl)phenyl]-5-[4-(3-methoxypropyl)-3,4-dihydro-2H-benzo[1,4]oxazin-6-ylmethoxy]piperidin-3-yloxy}-propan-2-ol

Starting from (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 48a).

### Example 23

### (R)-1-Methoxy-3-{(3S,4R,5R)-4-[4-(2-methoxyethyl)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.08 g of (R)-1-methoxy-3-[(3S,4R,5R)-4-[4-(2-methoxyethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting materials are prepared as follows:

### a) (R)-1-Methoxy-3-[(3S,4R,5R)-4-[4-(2-methoxyethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol

0.106 g of (3S,4S,5R)-4-[4-(2-methoxyethyl)phenyl]-5-[4-(3-methoxypropyl)-3,4-dihydro-2H-benzo[1,4]oxazin-6-ylmethoxy]-1-(toluene-4-sulphonyl)piperidin-3-ol and 0.032 g of (S)-(+)-glycidyl methyl ether [64491-68-5] are reacted in analogy to method M. The title compound is obtained as a colourless oil. Rf=0.17 (EtOAc/heptane 2:1); Rt=4.88 (gradient I).

### b) (3S,4S,5R)-4-[4-(2-Methoxyethyl)phenyl]-5-[4-(3-methoxypropyl)-3,4-dihydro-2H-benzo[1,4]oxazin-6-ylmethoxy]-1-(toluene-4-sulphonyl)piperidin-3-ol

0.155 g of 6-[(3R,4R,5S)-4-[4-(2-methoxyethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine is reacted in analogy to method J. The title compound is obtained as a colourless resin. Rf=0.11 (EtOAc/heptane 1:1); Rt=4.79 (gradient I).

### c) 6-[(3R,4R,5S)-4-[4-(2-Methoxyethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.032 g of sodium hydride (60% dispersion in oil) is added to a solution of 0.261 g of 2-{4-[(3R,4R,5S)-3-[4-(3-methoxypropyl)-3,4-dihydro-2H-benzo[1,4]oxazin-6-ylmethoxy]-1-(toluene-4-sulphonyl)-5-triisopropylsilanyloxypiperidine-4-yl]phenyl}ethanol and 0.232 ml of methyl iodide in 1 ml of N,N-dimethylformamide and 3 ml of tetrahydrofuran. After 6 hours at room temperature, the reaction mixture is diluted with 250 ml of tert-butyl methyl ether and washed successively with 50 ml of saturated sodium bicarbonate solution, 50 ml of water and 30 ml of brine, dried with sodium sulphate and evaporated. The title compound is obtained as a yellow oil from the residue by flash chromatography (SiO₂ 60F). Rf=0.60 (EtOAc/heptane 1:1).

### d) 2-{4-[(3R,4R,5S-3-[4-(3-Methoxypropyl)-3,4-dihydro-2H-benzo[1,4]oxazin-6-ylmethoxy]-1-(toluene-4-sulphonyl)-5-triisopropylsilanyloxypiperidin-4-yl]phenyl}ethanol

2.6 ml of diisobutylaluminium hydride (1N solution in dichloromethane) are added dropwise to a solution of 1.45 g of {4-[(3R,4R,5S)-3-[4-(3-methoxypropyl)-3,4-dihydro-2H-benzo[1,4]oxazin-6-ylmethoxy]-1-(toluene-4-sulphonyl-5-triisopropylsilanyloxypiperidin-4-yl]phenyl}acetonitrile in 15 ml of dichloromethane at −78° C. After 30 minutes at −78° C., the reaction mixture is stirred at room temperature for 2 hours and then quenched successively with 1N aqueous-ammonium chloride solution and with 1N aqueous HCl (pH 2). The mixture is extracted twice with 100 ml of tert-butyl methyl ether. The combined organic phases are washed with 30 ml of water and then 20 ml of brine, dried with sodium sulphate and evaporated. The residue is dissolved in 20 ml of tetrahydrofuran and, at 0° C., 2.88 ml of borane-THF complex (1M solution in tetrahydrofuran) are added. After 2 hours, 50 ml of methanol are cautiously added at 0° C., and the mixture is evaporated. The title compound is obtained as a yellowish oil from the residue by flash chromatography (SiO₂ 60F). Rf=0.21 (EtOAc/heptane 1:1).

### e) {4-[(3R,4R,5S-3-[4-(3-Methoxypropyl)-3,4-dihydro-2H-benzo[1,4]oxazin-6-ylmethoxy]-1-(toluene-4-sulphonyl)-5-triisopropylsilanyloxypiperidin-4-yl]phenyl}acetonitrile

0.072 g of tetrabutylammonium cyanide, 0.069 g of 18-crown-6 and 0.258 g of potassium cyanide are added to a solution of 2.0 g of 6-[(3R,4R,5S)-4-(4-chloromethylphenyl) 1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) in 20 ml of tetrahydrofuran and 3 ml of acetonitrile. After 2 hours at 50° C., the reaction mixture is diluted at room temperature with 250 ml of tert-butyl methyl ether. The mixture is washed successively with 20 ml of saturated sodium bicarbonate solution, 20 ml of water and 20 ml of brine, dried with sodium sulphate and evaporated. The title compound is obtained as a colourless oil from the residue by flash chromatography (SiO₂ 60F). Rf=0.25 (EtOAc/heptane 1:2).

### Example 24

### 6-[(3R,4S,5S)-4-[4-(2-Methoxyethoxymethyl)phenyl]-5-(3-methoxypropoxy)piperidin-3-yloxymethyl]-4-(3-methoxypropyl)-3,4-dihydro-2H-benzo[1,4]oxazine

The title compound is prepared from 0.281 mmol of 6-[(3R,4S,5S)-4-[4-(2-methoxyethoxymethyl)phenyl]-5-(3-methoxypropoxy)-1-(toluene-4-sulphonyl)piperidin-3-yloxymethyl]-4-(3-methoxypropyl)-3,4-dihydro-2H-benzo[1,4]oxazine in analogy to method L.

The starting material is prepared as follows:

### a) 6-[(3R,4S,5S)-4-[4-(2-Methoxyethoxymethyl)phenyl]-5-(3-methoxypropoxy)-1-(toluene-4-sulphonyl)piperidin-3-yloxymethyl]-4-(3-methoxypropyl)-3,4-dihydro-2H-benzo[1,4]oxazine

0.83 mmol of sodium hydride (60% dispersion in oil) is added to a solution of 0.55 mmol of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b), 0.69 mmol of 1-bromo-3-methoxypropane and 0.055 mmol of sodium iodide in 2 ml of N,N-dimethylformamide at −5° C., and the mixture is stirred at room temperature for 4 hours. the reaction mixture is poured into ice-water and extracted with tert-butyl methyl ether (3×). The combined organic phases are washed with water and brine, dried with sodium sulphate and evaporated. The title compound is obtained as a yellow oil from the residue by flash chromatography (SiO₂ 60F). Rf=0.26 (EtOAc/heptane 3:1); Rt=5.30 (gradient I).

### Example 25

### 6-[(3R,4S,5S)-4-[4-((S)-3-Methoxy-2-methylpropoxymethyl)phenyl]-5-(3-methoxypropoxy)-piperidin-3-yloxymethyl]-4-(3-methoxypropyl)-3,4-dihydro-2H-benzo[1,4]oxazine

The title compound is prepared from 6-[(3R,4R,5S)-4-(4-chloromethylphenyl)-1-(toluene-4-sulphonyl)-5-triisopropylsilanyloxypiperidin-3-yloxymethyl]-4-3-methoxypropyl-3,4-dihydro-2H-benzo[1,4]oxazine (Example 4c) in analogy to the process described in Example 24 and Example 5.

### Example 26

### 6-[(3R,4S,5S)-4-(4-Cyclopropylmethoxymethylphenyl)-5-(3-methoxypropoxy)piperidin-3-yloxymethyl]-4-(3-methoxypropyl)-3,4-dihydro-2H-benzo[1,4]oxazine

The title compound is prepared from 0.247 g of 6-[(3R,4S,5S)-4-(4-cyclopropylmethoxy-methylphenyl)-5-(3-methoxypropoxy)-1-(toluenesulphonyl)piperidin-3-yloxymethyl]-4(3-methoxypropyl)-3,4-dihydro-2H-benzo[1,4]oxazine in analogy to method L.

The starting material is prepared as follows:

### a) 6-[(3R,4S,5S)-4-(4-Cyclopropylmethoxymethylphenyl)-5-(3-methoxypropoxy)-1-(toluene-4-sulphonyl)piperidin-3-yloxymethyl]-4-(3-methoxypropyl)-3,4-dihydro-2H-benzo[1,4]oxazine

31 mg of sodium hydride (60% dispersion in oil) are added to a solution of 0.35 g of (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b), 0.133 g 1-bromo-3-methoxypropane and 8 mg of sodium iodide in 2 ml of N,N-dimethylformamide at −5° C., and the mixture is stirred at room temperature for 4 hours. The reaction mixture is poured into ice-water and extracted with tert-butyl methyl ether. The organic phases are washed with water and brine, dried with sodium sulphate and evaporated. The title compound is obtained as a colourless oil from the residue by flash chromatography (SiO₂ 60F). Rf=0.23 (EtOAc/heptane 3:1); Rt=5.73 (gradient I).

### Example 27

### (S)-1-Methoxy-3-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is prepared from 0.208 g of (S)-1-methoxy-3-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting material is prepared as follows:

### a) (S)-1-Methoxy-3-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-yloxy]propan-2-ol

0.20 g of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) and 0.060 g of (R)-(−)-glycidyl methyl ether [64491-70-9] are reacted in analogy to method M. The title compound is obtained as a yellow oil. Rf=0.05 (EtOAc/heptane 2:1); Rt=4.76 (gradient I).

### Example 28

### 6-[(3R,4R,5S)-4-[4-(2-Methoxyethoxymethyl)phenyl]-5-(2-[1,2,4]triazol-1-yl-ethoxy)piperidin-3-yloxymethyl]-4-(3-methoxypropyl)-3,4-dihydro-2H-benzo[1,4]oxazine

The title compound is prepared from 0.180 g of benzyl (3R,4R,5S)-4-[4-(2-methoxy-ethoxymethyl)phenyl]-3-[4-(3-methoxypropyl)-3,4-dihydro-2H-benzo[1,4]oxazin-6-ylmethoxy]-5-(2-[1,2,4]triazol-1-yl-ethoxy)piperidine-1-carboxylate in analogy to method B.

The starting materials are prepared as follows:

### a) Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-(2-[1,2,4]triazol-1-yl-ethoxy)piperidine-1-carboxylate

0.146 g of 1,2,4-triazole sodium salt [41253-21-8] is added to a solution of 0.240 g of benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-[2-(toluene-4-sulphonyloxy)ethoxy]piperidine-1-carboxylate in 6 ml of N,N-dimethylformamide at 0° C., and the mixture is then stirred at room temperature for 4 hours. The reaction mixture is poured into ice-water and extracted with tert-butyl methyl ether. The organic phases are washed with water and brine, dried with sodium sulphate and evaporated. The title compound is obtained as a colourless oil from the residue by flash chromatography (SiO₂ 60F). Rf=0.40 (dichloromethane/methanol/25% conc. ammonia=200:20:1); Rt=4.49 (gradient I).

### b) (3R,4R,5S)-4-[4-(2-Methoxyethoxymethyl)phenyl]-3-[4-(3-methoxypropyl)-3,4-dihydro-2H-benzo[1,4]oxazin-6-ylmethoxy]-5-[2-(toluene-4-sulphonyloxy)ethoxy]piperidine-1-carboxylate

0.815 g of benzyl (3S,4R,5R-3-(2-hydroxyethoxy)-4-[4-(2-methoxyethoxymethyl)phenyl]-5-[4-(3-methoxypropyl)-3,4-dihydro-2H-benzo[1,4]oxazin-6-ylmethoxy]piperidine-1-carboxylate is reacted in analogy to method H. The title compound is obtained as a yellowish oil. Rf=0.16 (EtOAc/heptane 2:1); Rt=5.51 (gradient I).

### c) Benzyl (3S,4R,5R-3-(2-Hydroxyethoxy)-4-[4-(2-methoxyethoxymethyl)phenyl]-5-[4-(3-methoxypropyl)-3,4-dihydro-2H-benzo[1,4]oxazin-6-ylmethoxy]piperidine-1-carboxylate

1.14 g of benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-(2-triisopropylsilanyloxyethoxy)piperidine-1-carboxylate are reacted in analogy to method J. The title compound is obtained as a yellowish oil. Rf=0.38 (EtOAc/heptane 2:1); Rt=4.63 (gradient I).

### d) Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-(2-triisopropylsilanyloxyethoxy)piperidine-1-carboxylate

0.165 g of sodium hydride (60% dispersion in oil) is added to a solution of 1.65 g of benzyl (3S,4S,5R)-3-hydroxy-4-[4-(2-methoxyethoxymethyl)phenyl]-5-[4-(3-methoxypropyl-3,4-dihydro-2H-benzo[1,4]oxazin-6-ylmethoxy]piperidine-1-carboxylate at 0° C., and the mixture is stirred for 30 minutes. 1.11 g of (2-iodoethoxy)triisopropylsilane are added to the resulting solution, and it is then stirred at room temperature for 14 hours. The reaction mixture is poured into ice-water and extracted with tert-butyl methyl ether. The organic phases are washed with water and brine, dried with sodium sulphate and evaporated. The title compound is obtained as a colourless oil from the residue by flash chromatography (SiO₂ 60F). Rf=0.39 (EtOAc/heptane 2:1).

### e) Benzyl (3S,4S,5R)-3-hydroxy-4-[4-(2-methoxyethoxymethyl)phenyl]-5-[4-(3-methoxypropyl)-3,4-dihydro-2H-benzo[1,4]oxazin-6-ylmethoxy]piperidine-1-carboxylate

Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-triisopropylsilanyloxypiperidine-1-carboxylate is reacted in analogy to method J. The title compound is obtained as a yellowish resin. Rf=0.30 (EtOAc/heptane 2:1); Rt=4.63 (gradient I).

### f) 2-Methoxyethyl (3R,4R,5S)-4-[4-(2-methoxyethoxymethyl)phenyl]-3-[4-(3-methoxypropyl-3,4-dihydro-2H-benzo[1,4]oxazin-6-ylmethoxy]-5-triisopropylsilanyloxypiperidine-1-carboxylate

and

### Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-triisopropylsilanyloxypiperidine-1-carboxylate

The two title compounds are obtained from 4.650 g of benzyl (3R,4R,5S)-4-(4-chloromethylphenyl)-3-[4-(3-methoxypropyl)-3,4-dihydro-2H-benzo[1,4]oxazin-6-ylmethoxy]-5-triisopropylsilanyloxypiperidine-1-carboxylate in analogy to method D.
2-Methoxyethyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-triisopropylsilanyloxypiperidine-1-carboxylate:
Yellowish resin; Rf=0.26 (EtOAc/heptane 1:1); Rt=29.90 (gradient II).
Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-triisopropylsilanyloxypiperidine-1-carboxylate:
Yellowish resin; Rf=0.36 (EtOAc/heptane 1:1); Rt=31.96 (gradient II).

### g) Benzyl (3R,4R,5S)-4-(4-chloromethylphenyl)-3-[4-(3-methoxypropyl)-3,4-dihydro-2H-benzo[1,4]oxazin-6-ylmethoxy]-5-triisopropylsilanyloxypiperidine-1-carboxylate

A solution of 5.430g of benzyl (3R,4R,5S)-4-(4-hydroxyethylphenyl)-3-[4-(3-methoxypropyl)-3,4-dihydro-2H-benzo[1,4]oxazin-6-ylmethoxy]-5-triisopropylsilanyloxypiperidine-1-carboxylate (Example 4f) in 100 ml of dichloromethane is cooled to 0° C., and 12.12 ml of 1-chloro-N,N,2-trimethylpropenylamine are added dropwise. The reaction solution is warmed to 20° C. over 16 hours, tert-butyl methyl ether and water are added, and the phases are separated. The organic phase is washed with brine, dried (sodium sulphate) and evaporated. The title compound is obtained as a yellowish oil from the residue by flash chromatography (SiO₂ 60F). Rf=0.39 (EtOAc/heptane 1:2).

### Example 29

### (2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}ethyl)dimethylamine

The title compound is prepared from 0.215 g of benzyl (3S,4R,5R)-3-(2-dimethylamino-ethoxy)-4-[4-(2-methoxyethoxymethyl)phenyl]-5-[4-(3-methoxypropyl)-3,4-dihydro-2H-benzo-[1,4]oxazin-1-ylmethoxy]piperidine-1-carboxylate in analogy to method B.

The starting material is prepared as follows:

### a) Benzyl (3S,4R,5R)-3-(2-dimethylaminoethoxy)-4-[4-(2-methoxyethoxymethyl)phenyl]-5-[4-(3-methoxypropyl)-3,4-dihydro-2H-benzo[1,4]oxazin-6-ylmethoxy]piperidine-1-carboxylate

A solution of 0.290 g of benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-[2-(toluene-4-sulphonyloxy)ethoxy]piperidine-1-carboxylate (Example 28a), 0.24 ml of triethylamine and 3.13 ml of dimethylamine (33% in ethanol) is stirred at room temperature for 3 hours. The reaction mixture is then poured into ice-water and extracted with tert-butyl methyl ether. The organic phases are washed with water and brine, dried with sodium sulphate and evaporated. The title compound is obtained as a yellowish oil from the residue by flash chromatography (SiO₂ 60F). Rf=0.17 (dichloromethane/methanol/25% conc. ammonia=200:20:1); Rt=4.33 (gradient I).

### Example 30

### 6-[(3R,4S,5S)-4-[4-(2-Methoxyethoxymethyl)phenyl]-5-(3-[1,2,4]triazol-1-yl-propoxy)-piperidin-3-yloxymethyl]-4-(3-methoxypropyl)-3,4-dihydro-2H-benzo[1,4]oxazine

The title compound is prepared from 0.062 g of 6-[(3R,4S,5S)-4-[4-(2-methoxyethoxymethyl)phenyl]-1-(toluene-4-sulphonyl)-5-(3-[1,2,4]triazol-1-yl-propoxy)piperidin-3-yloxymethyl]-4-(3-methoxypropyl)-3,4-dihydro-2H-benzo[1,4]oxazine in analogy to method L.

The starting materials are prepared as follows:

### a) 6-[(3R,4S,5S)-4-[4-2-Methoxyethoxymethyl)phenyl]-1-(toluene-4-sulphonyl)-5-(3-[1,2,4]-triazol-1-yl-propoxy)piperidin-3-yloxymethyl]-4-3-methoxypropyl)-3,4-dihydro-2H-benzo-[1,4]oxazine

The title compound is obtained as a yellowish oil from 0.099 g of 3-[(3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-yloxy]propyl toluene-4-sulphonate in analogy to Example 28a. Rf=0.19 (dichloromethane/methanol 95:5); Rt=4.70 (gradient I).

### b) 3-[(3S,4S,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propyl toluene-4-sulphonate

The title compound is obtained as a colourless oil from 0.107 g of 3-[(3S,4S,5R)-4-[4-2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-1-ol in analogy to method H. Rf=0.34 (EtOAc/heptane 3:1); Rt=5.63 (gradient I).

### c) 3-[(3S,4S,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-1-ol

The title compound is obtained as a colourless oil from 0.177 g of 3-[(3R,4S,5S)-4-[4-(2-methoxyethoxymethyl)phenyl]-1-(toluene-4-sulphonyl)-5-(3-triisopropylsilanyloxypropoxy)-piperidin-3-yloxymethyl]-4-(3-methoxypropyl)-3,4-dihydro-2H-benzo[1,4]oxazine in analogy to method J. Rf=0.07 (EtOAc/heptane 4:1); Rt=4.75 (gradient I).

### d) 3-[(3R,4S,5S)-4-[4-2-Methoxyethoxymethyl)phenyl]-1-(toluene-4-sulphonyl)-5-(3-triisopropylsilanyloxypropoxy)piperidin-3-yloxymethyl]-4-(3-methoxypropyl)-3,4-dihydro-2H-benzo[1,4]oxazine

0.030g of sodium hydride (60% dispersion in oil) is added to a solution of 0.324 g of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) in 3 ml of N,N-dimethylformamide at 0° C. The reaction mixture is stirred at room temperature for 30 minutes and then 0.008 g of sodium iodide and 0.221 g of (3-bromopropoxy)triisopropylsilane [215650-24-1] are added. The reaction mixture is stirred at room temperature for 2 hours. The reaction mixture is poured into saturated aqueous sodium bicarbonate solution, and the mixture is extracted with tert-butyl methyl ether. The combined organic extracts are washed with brine, dried with sodium sulphate and evaporated. The title compound is obtained as a yellowish oil from the residue by flash chromatography (SiO₂ 60F). Rf=0.49 (EtOAc/heptane 2:1); Rt=32.67 (gradient II).

### Example 31

### (R)-1{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}propan-2-01

The title compound is obtained from 0.262 g of benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate in analogy to method B.

The starting material is prepared as follows:

### a) Benzyl (3R,4R,5S)-4-[4-(2-methoxyethoxymethyl)phenyl]-3-[4-(3-methoxypropyl)-3,4-dihydro-2H-benzo[1,4]oxazin-6-ylmethoxy]-5-((R)-1-oxiranylmethoxy)piperidine-1-carboxylate

0.043 g of sodium hydride (60% dispersion in oil) is added to a solution of 0.507 g of benzyl (3S,4S,5R)-3-hydroxy-4-[4-(2-methoxyethoxymethyl)phenyl]-5-[4-(3-methoxypropyl)-3,4-di-hydro-2H-benzo[1,4]oxazin-6-ylmethoxy]piperidine-1-carboxylate (Example 28e) in 5 ml of tetrahydrofuran. The mixture is stirred at 40° C. for 45 minutes. A solution of 0.354 g of (R)-1-oxiranylmethyl toluene-4-sulphonate [113826-06-5] in 3 ml tetrahydrofuran is added, and the mixture is heated at 50° C. for 3 hours. The reaction mixture is poured into saturated aqueous sodium bicarbonate solution, and the mixture is extracted with tert-butyl methyl ether. The combined organic extracts are washed with brine, dried over sodium sulphate and evaporated. The title compound is obtained as a colourless oil from the residue by flash chromatography (SiO₂ 60F). Rf=0.24 (EtOAc/heptane 2:1); Rt=5.25 (gradient I).

The following compounds are prepared in an analogous manner to the process described in Example 31:

### Examples

### 33 (S)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}propan-2-ol

### 49 (R)-1-{(3S,4R,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-[4-(3-methoxypropoxymethyl)phenyl]-5-[4-(3-methoxypropyl, 3,4-dihydro-2H-benzo[1,4]oxazin-6-ylmethoxy]-1-(toluene-4-sulphonyl)piperidin-3-ol (Example 48a). Deprotection of the protective group on the nitrogen (last stage of the synthesis) is carried out in analogy to method L.

### 76 (S)-1-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b). Deprotection of the protective group on the nitrogen (last stage of the synthesis) is carried out in analogy to method L.

### 77 (S)-1-{(3S,4R,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-[4-(3-methoxypropoxymethyl)phenyl]-5-[4-(3-methoxypropyl) 3,4-dihydro-2H-benzo[1,4]oxazin-6-ylmethoxy]-1-(toluene-4-sulphonyl)piperidin-3-ol (Example 48a). Deprotection of the protective group on the nitrogen (last stage of the synthesis) is carried out in analogy to method L.

### 80 (S)-1-{(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-[4-((S-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 5a). Deprotection of the protective group on the nitrogen (last stage of the synthesis) is carried out in analogy to method L.

### 112 (R)-1-{(3S,4R,5R)-4-[4-(1-Methoxymethylcyclopropylmethoxymethyl)phenyl]-5-[4-(3-methoxypropyl)-3,4-dihydro-2H-benzo[1,4]oxazin-6-ylmethoxy]piperidin-3-yloxy}-propan-2-ol

Starting from (3S,4S,5R)-4-[4-(1-methoxymethylcyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol. Deprotection of the protective group on the nitrogen (last stage of the synthesis) is carried out in analogy to method L.

The starting materials are prepared as follows:

### a) (3S,4S,5R)-4-[4-(1-Methoxymethylcyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

The title compound is identified on the basis of the Rf from 0.5 mmol of 6-[(3R,4R,5S)-4-[4-(1-methoxymethylcyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo-[1,4]oxazine in analogy to method J.

### b) 6-[(3R,4R,5S)-4-[4-(1-Methoxymethylcyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

The title compound is identified on the basis of the Rf from 1 mmol of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and (1-methoxymethylcyclopropyl)methanol [338455-22-4] in analogy to example 4b.

### 113 (R)-1-{(3S,4R,5R)-4-[4-(1-Methoxycyclopropylmethoxymethyl)phenyl]-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-[4-(1-methoxycyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol. Deprotection of the protective group on the nitrogen (last stage of the synthesis) is carried out in analogy to method L.

The starting materials are prepared as follows:

### a) (3S,4S,5R)-4-[4-(1-Methoxycyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

The title compound is prepared in analogy to the process described in Example 112 from 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and (1-methoxycyclopropyl)methanol and identified on the basis of the Rf.

### b) (1-Methoxycyclopropyl)methanol

The title compound is identified on the basis of the Rf from 2 mmol of methyl 1-methoxy-cyclopropanecarboxylate in analogy to example 10e.

### Example 32

### 2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-1-pyrrolidin-1-ylethanone

The title compound is prepared from 0.121 g of 2-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]-1-pyrrolidin-1-ylethanone in analogy to method L.

The starting materials are prepared as follows:

### a) 2-[(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]-1-pyrrolidin-1-ylethanone

0.194 ml of propylphosphonic anhydride [68957-94-8, T3P] (50% in ethyl acetate) is added to a solution of 0.196 g of [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]acetic acid, 0.024 g of pyrrolidine and 0.193 ml of triethylamine in 2 ml of dichloromethane at 0° C., and the mixture is stirred at room temperature for 16 hours. The reaction mixture is diluted with dichloromethane, and 0.1M aqueous HCl is added. The phases are separated and the aqueous phase is extracted twice more with dichloromethane. The combined organic phases are washed with brine, dried with sodium sulphate and evaporated. The title compound is obtained as a yellow oil from the residue by flash chromatography (SiO₂ 60F). Rf=0.17 (EtOAc); Rt=4.86 (gradient I).

### b) [(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy] acetic acid

4 ml of a 1.5M aqueous lithium hydroxide solution are added to a solution of 0.24 g of methyl [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy] acetate in 4 ml of tetrahydrofuran, and the mixture is stirred at room temperature for 5 hours. 2M aqueous HCl is added to the reaction mixture until the pH is 2. The resulting mixture is extracted twice with 80 ml of ethyl acetate each time. The combined organic phases are washed with brine, dried with sodium sulphate and evaporated. The title compound is obtained without further purification as a yellow oil. Rt=4.67 (gradient I).

### c) Methyl [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy] acetate

0.02 g of sodium hydride (60% dispersion in oil) is added to a solution of 0.25 g of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b), 0.241 g of methyl bromoacetate and 5.7 mg of sodium iodide in 3 ml of N,N-dimethylformamide at room temperature, and the mixture is stirred at room temperature for 3 hours. The reaction mixture is diluted with ethyl acetate and poured into 0.1M aqueous HCl. The resulting mixture is extracted three times with ethyl acetate. The combined organic phases are washed with brine, dried with sodium sulphate and evaporated. The title compound is obtained as a yellow oil from the residue by flash chromatography (SiO₂ 60F). Rt=5.11 (gradient I).

The following compounds are prepared in an analogous manner to the process described in Example 32:

### Examples

### 35 N,N-Diethyl-2-{(3S,4R,5R)4-[4-(2-methoxyethoxymethyl)phenyl]-5-[4-(3-methoxypropyl)-3,4-dihydro-2H-benzo[1,4]oxazin-6-ylmethoxy]piperidin-3-yloxy}acetamide

### 36 N-Ethyl-2-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-yloxy}-N-methylacetamide

### 37 2-{(3S,4R,5R)-4-[4-(2-Methoxyethoxymethyl)phenyl]-5-[4-(3-methoxypropyl) 3,4-dihydro-2H-benzo[1,4]oxazin-4-ylmethoxy]piperidin-3-yloxy}-N-methyl-N-propylacetamide

### 38 2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-N-propylacetamide

### 39 2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-N,N-dimethylacetamide

### 65 2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-piperidin-1-ylethanone

### 66 2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-1-((R)-2-methylpyrrolidin-1-yl)ethanone

The following compounds are prepared in analogous manner to the process described in Example 32 starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b):

### Examples

### 46 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-N,N-dimethylacetamide

### 47 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-1-pyrrolidin-1-yl-ethanone

### 56 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-N-propylacetamide

### 58 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-N,N-diethyl-acetamide

### 59 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-N-ethyl-N-methylacetamide

### 62 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-1-((R)-2-methyl-pyrrolidin-1-yl)ethanone

### 63 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-piperidin-1-ylethanone

### 64 2-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}-1-morpholin-4-ylethanone

The following compounds are prepared in an analogous manner to the process described in Example 32 starting from (3S,4S,5R)-4-(4-methoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 21b):

### Examples

### 53 2-{(3S,4R,5R)-4-(4-Methoxymethylphenyl)-5-[4-(3-methoxypropyl)-3,4-dihydro-2H-benzo[1,4]oxazin-6-ylmethoxy]piperidin-3-yloxy}-1-((R-2-methylpiperidin-1-yl)ethanone

### 54 1-((2S,6R)-2,6-Dimethylpiperidin-1-yl)-2-{(3S,4R,5R)-4-(4-methoxymethylphenyl)-5-[4-(3-methoxypropyl)-3,4-dihydro-2H-benzo[1,4]oxazin-6-ylmethoxy]piperidin-3-yl-oxy}ethanone

### 55 2-{(3S,4R,5R)-4-(4-Methoxymethylphenyl)-5-[4-(3-methoxypropyl)-3,4-dihydro-2H-benzo[1,4]oxazin-6-ylmethoxy]piperidin-3-yloxy}-1-piperidin-1-ylethanone

### 57 2-{(3S,4R,5R)-4-(4-Methoxymethylphenyl)-5-[4-(3-methoxypropyl)-3,4-dihydro-2H-benzo[1,4]oxazin-6-ylmethoxy]piperidin-3-yloxy}-1-pyrrolidin-1-ylethanone

### 60 2-{(3S,4R,5R)-4-(4-Methoxymethylphenyl)-5-[4-(3-methoxypropyl)-3,4-dihydro-2H-benzo[1,4]oxazin-6-ylmethoxy]piperidin-3-yloxy}-1-((R)-3-methylmorpholin-4-yl)-ethanone

### 61 1-((3S,5R)-3,5-Dimethylmorpholin-4-yl)-2-{(3S,4R,5R)-4-(4-methoxymethylphenyl)-5-[4-(3-methoxypropyl)-3,4-dihydro-2H-benzo[1,4]oxazin-6-ylmethoxy]piperidin-3-yloxy}ethanone

### 73 N-Ethyl-2-{(3S,4R,5R)-4-(4-methoxymethylphenyl)-5-[4-(3-methoxypropyl)-3,4-dihydro-2H-benzo[1,4]oxazin-6-ylmethoxy]piperidin-3-yloxy}-N-methylacetamide

The following compounds are prepared in an analogous manner to the process described in Example 32 starting from (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 48a):

### Examples

### 90 2-{(3S,4R,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}-1-pyrrolidin-1-yl-ethanone

### 91 N,N-Diethyl-2-{(3S,4R,5R)-4-[4-(3-methoxypropoxymethyl)phenyl]-5-[4-(3-methoxypropyl)-3,4-dihydro-2H-benzo[1,4]oxazin-6-ylmethoxy]piperidin-3-yloxy}acetamide

### 92 N-Ethyl-2-{(3S,4R,5R)-4-[4-(3-methoxypropoxymethyl)phenyl]-5-[4-(3-methoxypropyl)-3,4-dihydro-2H-benzo[1,4]oxazin-6-ylmethoxy]piperidin-3-yloxy}-N-methylacetamide

### Example 34

### 6-[(3R,4R,5S)-4-[4-(2-Methoxyethoxymethyl)phenyl]-5-(3-methyl-3H-imidazol-4-ylmethoxy)piperidin-3-yloxymethyl]-4-(3-methoxypropyl)-3,4-dihydro-2H-benzo[1,4]oxazine

2-Methoxyethyl (3R,4R,5S)-4-[4-(2-methoxyethoxymethyl)phenyl]-3-[4-(3-methoxypropyl) 3,4-dihydro-2H-benzo[1,4]oxazin-6-ylmethoxy]-5-(3-methyl-3H-imidazol-4-ylmethoxy)-piperidine-1-carboxylate (0.150 g) is dissolved in 1:1 methanol/dioxane (4 ml), and 2 ml of 40% aqueous potassium hydroxide solution are added to the solution. The mixture is heated in a closed flask at 80° C. for 4 hours. The reaction solution is poured into water and extracted with tert-butyl methyl ether. The combined organic extracts are washed with brine, dried over sodium sulphate and concentrated. The title compound is obtained from the residue by flash chromatography (SiO₂ 60F).

The starting material is prepared as follows:

### a) 2-Methoxyethyl (3R,4R,5S)-4-[4-(2-methoxyethoxymethyl)phenyl]-3-[4-(3-methoxy-propyl)-3,4-dihydro-2H-benzo[1,4]oxazin-6-ylmethoxy]-5-(3-methyl-3H-imidazol-4-ylmethoxy)piperidine-1-carboxylate

0.086 g of sodium hydride (60% dispersion in oil) is added to a solution of 0.430 g of 2-methoxyethyl (3S,4S,5R)-3-hydroxy-4-[4-(2-methoxyethoxymethyl)phenyl]-5-[4-(3-methoxypropyl)-3,4-dihydro-2H-benzo[1,4]oxazin-6-ylmethoxy]piperidine-1-carboxylate (Example 28e) and 0.241 g of 5-chloromethyl-1-methyl-1H-imidazole hydrochloride [90773-41-4] in 4 ml of N,N-dimethylformamide at 0° C. 0.027 g of tetrabutylammonium iodide is added, and the reaction mixture is stirred at room temperature for 18 hours. The reaction mixture is poured into saturated aqueous sodium bicarbonate solution, and the mixture is extracted with tert-butyl methyl ether. The combined organic extracts are washed with brine, dried over sodium sulphate and evaporated. The title compound is obtained as a yellowish oil from the residue by flash chromatography (SiO₂ 60F). Rf=0.11 (dichloromethane/methanol 95:5); Rt=3.79. (gradient I).

### Example 40

### (R)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}butan-2-ol

The title compound is obtained from 0.726 g of (R)-1-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-toluene-3-sulphony)piperidin-3-yloxy]butan-2-ol in analogy to method L.

The starting material is prepared as follows:

### a) (R)-1-[(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-toluene-3-sulphonyl)piperidin-3-yloxy]-butan-2-ol

0.015 g of copper(I) cyanide is taken up in 10 ml of dry tetrahydrofuran under argon in a heat-dried Schlenk tube. The suspension is cooled to −78° C., and 0.429 ml of methyl-magnesium bromide solution (35% in diethyl ether) is added dropwise. A solution of 0.815 g of 6-[(3R,4R,5S)-4-[4-(2-methoxyethoxymethyl)phenyl]-5-((R)-1-oxiranylmethoxy-1-(toluene-4-sulphonyl)piperidinyloxymethyl]-4-(3-methoxypropyl)-3,4-dihydro-2H-benzo[1,4]oxazine in 5 ml of dry tetrahydrofuran is added, and the reaction mixture is stirred at −78° C. for 30 minutes and then thawed to 20° C. over 16 hours. The reaction mixture is poured into saturated aqueous ammonium chloride solution and adjusted to pH 10 with 25% ammonium hydroxide solution. The mixture is extracted with diethyl ether, and the combined organic extracts are washed with brine, dried with sodium sulphate and evaporated. The title compound is obtained as a yellow resin from the residue by flash chromatography (SiO₂ 60F). Rf=0.14 (EtOAc/heptane 2:1); Rt=5.06 (gradient I).

### b) 6-[(3R,4R,5S)-4-[4-(2-Methoxyethoxymethyl)phenyl]-5-((R)-1-oxiranylmethoxy)-1-(toluene-4-sulphonyl)piperidinyloxymethyl]-4-(3-methoxypropyl)-3,4-dihydro-2H-benzo[1,4]oxazine

The title compound is obtained as a colourless oil from (3S,4S,5R)-4-[4-(2-methoxyethoxymethyl)phenyl]-5-[4-(3-methoxypropyl)-3,4-dihydro-2H-benzo[1,4]oxazin-6-yloxymethyl]-1-(toluene-4-sulphonyl)piperidin-3-ol (Example 22b) in analogy to Example 31a. Rf=0.13 (EtOAc/heptane 3:1); Rt=5.09 (gradient I).

The following compounds are prepared in an analogous manner to the process described in Example 40:

### Examples

### 78 (S)-1{(3S,4R,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-(3-methoxypropoxymethyl)phenyl]-5-[4-(3-methoxypropyl) 3,4-dihydro-2H-benzo[1,4]oxazin-6-ylmethoxy]-1-(toluene-4-sulphonyl)piperidin-3-ol (Example 48a).

### 79 (S)-1-{(3S,4R,5R)-4-[4-(3-Methoxypropoxymethyl)phenyl]-5-[4-(3-methoxypropyl) 3,4-dihydro-2H-benzo[1,4]oxazin-6-ylmethoxy]piperidin-3-yloxy}pentan-2-01

Starting from (3S,4S,5R)-4-[4-(3-methoxypropoxymethyl)phenyl]-5-[4-(3-methoxypropyl) 3,4-dihydro-2H-benzo[1,4]oxazin-6-ylmethoxy]-1-(toluene-4-sulphonyl)piperidin-3-ol (Example 48a) using ethylmagnesium bromide solution.

### 81 (R)-1-{(3S,4R,5R)-4-[4-(3-Methoxypropoxymethyl)phenyl]-5-[4-(3-methoxypropyl) 3,4-dihydro-2H-benzo[1,4]oxazin-6-ylmethoxy]piperidin-3-yloxy}butan-2-ol

Starting from (3S,4S,5R)-4-[4-(3-methoxypropoxymethyl)phenyl]-5-[4-(3-methoxypropyl) 3,4-dihydro-2H-benzo[1,4]oxazin-6-ylmethoxy]-1-(toluene-4-sulphonyl)piperidin-3-ol (Example 48a).

### 86 (R)-1-{(3S,4R,5R)-4-(4-Cyclopropylmethoxymethylphenyl)-5-[4-(3-methoxypropyl) 3,4-dihydro-2H-benzo[1,4]oxazin-6-ylmethoxy]piperidin-3-yloxy}butan-2-ol

Starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b).

### 87 (S)-1-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}butan-2-01

Starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b).

### 93 (R)-1-{(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 5a).

### 94 (S)-1-{(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-((S)-3-methoxy-2-methylpropoxymethyl)phenyl]-5-[4-(3-methoxypropyl-3,4-dihydro-2H-benzo[1,4]oxazin-6-ylmethoxy]-1-(toluene-4-sulphonyl)piperidin-3-ol (Example 5a).

### 96 (R)-1-{(3S,4R,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}pentan-2-ol

Starting from (3S,4S,5R)-4-[4-(3-methoxypropoxymethyl)phenyl]-5-[4-(3-methoxy-propyl)-3,4-dihydro-2H-benzo[1,4]oxazin-6-ylmethoxy]-1-(toluene-4-sulphonyl)piperidin-3-ol (Example 48a) using ethylmagnesium bromide solution.

### 98 (R)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}pentan-2-ol

Starting from (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) using ethylmagnesium bromide solution.

### 99 (S)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}pentan-2-ol

Starting from (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) using ethylmagnesium bromide solution.

### 110 (R)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b).

### 111 (S)-1-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b).

### Example 41

### (3S,4S,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ol

The title compound is prepared from 14.64 g of (3S,4S,5R)-4-[4-(2-methoxyethoxymethyl)-phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) in analogy to method L.

### Example 43

### (R)-1-{(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is prepared from 215 mg (R)-1-[(3S,4R,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting materials are prepared as follows:

### a) (R)-1-[(3S,4R,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol

42 mg of sodium borohydride are added to a solution of 275 mg of 6-[(3R,4R,5S)-4-[4-((S)-3-methoxy-2-methylpropoxymethyl)phenyl]-5-((R)-1-oxiranylmethoxy)-1-(toluene-4-sulphonyl)piperidin-3-yloxymethyl]-4-(3-methoxypropyl)-3,4-dihydro-2H-benzo[1,4]oxazine in 5 ml of ethanol and 0.25 ml of tetrahydrofuran. After 21 hours at 45° C., the reaction mixture is diluted with tert-butyl methyl ether. The mixture is washed successively with saturated ammonium chloride solution, water and brine. The combined aqueous phases are back-extracted with dichloromethane (1×). The combined organic phases are dried with sodium sulphate and evaporated. The title compound is obtained as a yellowish resin from the residue by flash chromatography (SiO₂ 60F). Rf=0.08 (EtOAc/heptane 1:1); Rt=5.34 (gradient I).

### b) 6-[(3R,4R,5S)-4-[4-((S)-3-Methoxy-2-methylpropoxymethyl)phenyl]-5-((R)-1-oxiranyl-methoxy)-1-(toluene-4-sulphonyl)piperidin-3-yloxymethyl]-4-(3-methoxypropyl)-3,4-dihydro-2H-benzo[1,4]oxazine

396 mg of (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 5a) and 334 mg of (R)-1-oxiranymethyl toluene-4-sulphonate [113826-06-5] are reacted in analogy to Example 31a. The title compound is obtained as a colourless resin. Rf=0.05 (EtOAc/heptane 1:2); Rt=5.49 (gradient I).

### Example 44

### (R)-1-{(3S,4R,5R)-5-[4-(3-Methoxypropyl)-3,4-dihydro-2H-benzo[1,4]oxazin-6-ylmethoxy]-4-[4-((R)-2-methyl-3-methylsulphanylpropoxymethyl)phenyl]piperidin-3-yloxy}propan-2-ol

The title compound is prepared from 565 mg of (R)-1-[(3S,4R,5R)-5-[4-(3-methoxypropyl)-3,4-dihydro-2H-benzo[1,4]oxazin-6-ylmethoxy]-4-[4-((R)-2-methyl-3-methylsulphanylpropoxymethyl)phenyl]-1-(toluene-4-sulphonyl)piperidin-3-yloxy]propan-2-ol in analogy to method N.

The starting materials are prepared as follows:

### a) (R)-1-[(3S,4R,5R)-5-[4-(3-Methoxypropyl)-3,4-dihydro-2H-benzo[1,4]oxazin-6-ylmethoxy]-4-[4-((R)-2-methyl-3-methylsulphanylpropoxymethyl)phenyl]-1-(toluene-4-sulphonyl)piperidin-3-yloxy]propan-2-ol

670 mg of 4-3-methoxypropyl)-6-[(3R,4R,5S)-4-[4-((R-2-methyl-3-methylsulphanylpropoxymethyl)phenyl]-5-((R)-1-oxiranylmethoxy)-1-(toluene-4-sulphonyl)piperidin-3-yloxymethyl]-3,4-dihydro-2H-benzo[1,4]oxazine are reacted in analogy to Example 43a. The title compound is obtained as a yellowish resin. Rf=0.09 (EtOAc/heptane 1:1); Rt=5.63 (gradient I).

### b) 4-(3-Methoxypropyl)-6-[(3R,4R,5S)-4-[4-((R)-2-methyl-3-methylsulphanylpropoxymethyl)phenyl]-5-((R)-1-oxiranylmethoxy)-1-(toluene-4-sulphonyl)piperidin-3-yloxymethyl]-3,4-dihydro-2H-benzo[1,4]oxazine

848 mg of (3S,4S,5R)-5-[4-(3-methoxypropyl)-3,4-dihydro-2H-benzo[1,4]oxazin-6-ylmethoxy]-4-[4-((R)-2-methyl-3-methylsulphanylpropoxymethyl)phenyl]-1-(toluene-4-sulphonyl)piperidin-3-ol (Example 9a) and 699 mg of (R)-1-oxiranylmethyl toluene-4-sulphonate [113826-06-5] are reacted in analogy to Example 31a. The title compound is obtained as a colourless resin. Rf=0.10 (EtOAc/heptane 1:2); Rt=5.79 (gradient I).

### Example 45

### (R)-1-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}propan-2-ol

The title compound is prepared from 210 mg of (R)-1-[(3S,4R,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]propan-2-ol in analogy to method L.

The starting materials are prepared as follows:

### a) (R)-1-[(3S,4R,5R)-4-(4-Cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-yloxy]-propan-2-ol

480 mg of 6-[(3R,4R,5S)-4-(4-cyclopropylmethoxymethylphenyl)-5-((R)-1-oxiranylmethoxy)-1-(toluene-4-sulphonyl)piperidin-3-yloxymethyl]-4-(3-methoxypropyl)-3,4-dihydro-2H-benzo-[1,4]oxazine are reacted in analogy to Example 43a. The title compound is obtained as a cloudy colourless oil. Rf=0.20 (EtOAc/heptane 3:1); Rt=5.27 (gradient I).

### b) 6-[(3R,4R,5S)-4-(4-Cyclopropylmethoxymethylphenyl)-5-((R)-1-oxiranylmethoxy)-1-(toluene-4-sulphonyl)piperidin-3-yloxymethyl]-4-(3-methoxypropyl)-3,4-dihydro-2H-benzo[1,4]oxazine

370 mg of (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b) and 256 mg of (R)-1-oxiranylmethyl toluene-4-sulphonate [113826-06-5] are reacted in analogy to Example 31a. The title compound is obtained as a yellow oil. Rf=0.50 (EtOAc/heptane 3:1); Rt=5.47 (gradient I).

### Example 48

### (3S,4S,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-ol

The title compound is prepared from 342 mg of (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol in analogy to method L.

The starting materials are prepared as follows:

### a) (3S,4S,5R)-4-[4-(3-Methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

1.18 g of 6-[(3R,4R,5S)-4-[4-(3-methoxypropoxymethyl)phenyl]-1-(toluene-4-sulphonyl-5-triisopropylsilanyloxypiperidin-3-yloxymethyl]-4-(3-methoxypropyl)-3,4-dihydro-2H-benzo-[1,4]oxazine are reacted in analogy to method J. The title compound is obtained as a yellow oil. Rf=0.3 (EtOAc/heptane 2:1); Rt=4.85 (gradient I).

### b) 6-[(3R,4R,5S)-4-[4-(3-Methoxypropoxymethyl)phenyl]-1-(toluene-4-sulphonyl)-5-triisopropylsilanyloxypiperidin-3-yloxymethyl]-4-(3-methoxypropyl)-3,4-dihydro-2H-benzo-[1,4]oxazine

2 g of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and 0.48 g of 3-methoxy-1-propanol are reacted in analogy to Example 4b. The title compound is obtained as a yellow oil. Rf=0.5 (EtOAc/heptane 1:1); Rt=29.43 (II).

### Example 50

### (3S,4S,5R)-5-[4-(3-Methoxypropyl)-3,4-dihydro-2H-benzo[1,4]oxazin-6-ylmethoxy]-4-[4-(2-methylsulphanylethoxymethyl)phenyl]piperidin-3-ol

The title compound is prepared from 0.20 mmol of (3S,4S,5R)-5-[4-(3-methoxypropyl)-3,4-dihydro-2H-benzo[1,4]oxazin-6-ylmethoxy]-4-[4-(2-methylsulphanylethoxymethyl)phenyl]-1-(toluene-4-sulphonyl)piperidin-3-ol in analogy to method N.

The starting material is prepared as follows:

### a) (3S,4S,5R-5-[4-(3-Methoxypropyl)-3,4-dihydro-2H-benzo[1,4]oxazin-6-ylmethoxy]-4-[4-(2-methylsulphanylethoxymethyl)phenyl]-1-(toluene-4-sulphonyl)piperidin-3-ol

0.8 mmol of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl-3,4-dihydro-2H-benzo[1,4]oxazine (Example 4c) and 1.0 mmol of 2-methylsulphanylethanol are reacted in analogy to Example 4b. The title compound is obtained as a yellow oil. Rf=0.18 (EtOAc/heptane 1:1); Rt=5.06 (gradient I).

The following compound is prepared in an analogous manner to the process described in Example 50.

### Example

### 51 (3S,4S,5R)-4-[4-(2-Methoxyethylsulphanylmethyl)phenyl]-5-[4-(3-methoxy-propyl)-3,4-dihydro-2H-benzo[1,4]oxazin-6-ylmethoxy]piperidin-3-ol

Starting from (3S,4S,5R)-4-[4-(2-methoxyethylsulphanylmethyl)phenyl]-5-[4-(3-methoxypropyl)-3,4-dihydro-2H-benzo[1,4]oxazin-6-ylmethoxy]-1-(toluene-4-sulphonyl)piperidin-3-ol.

The starting materials are prepared as follows:

### a) (3S,4S,5R)-4-[4-(2-Methoxyethylsulphanylmethyl)phenyl]-5-[4-(3-methoxypropyl)-3,4-dihydro-2H-benzo[l. 4]oxazin-6-ylmethoxy]-1-(toluene-4-sulphonyl)piperidin-3-ol

0.75 mmol of 6-[(3R,4R,5S)-4-[4-(2-methoxyethylsulphanylmethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine are reacted in analogy to method J. The title compound is identified on the basis of the Rf.

### b) 6-[(3R,4R,5S)-4-[4-(2-Methoxyethylsulphanylmethyl)phenyl]-1-(toluene-4-sulphonyl)-5-triisopropylsilanyloxypiperidin-3-yloxymethyl]-4-(3-methoxypropyl)-3,4-dihydro-2H-benzo-[1,4]oxazine

1.0 mmol of 2-{4-[(3R,4R,5S)-3-[4-(3-methoxypropyl)-3,4-dihydro-2H-benzo[1,4]oxazin-6-ylmethoxy]-1-(toluene-4-sulphonyl)-5-triisopropylsilanyloxypiperidin-4-yl]benzylsulphanyl}ethanol and 1.5 mmol of methyl iodide are reacted in analogy to method D. The title compound is identified on the basis of the Rf.

### c) 2-{4-[(3R,4R,5S)-3-[4-(3-Methoxypropyl)-3,4-dihydro-2H-benzo[1,4]oxazin-6-ylmethoxy]-1-(toluene-4-sulphonyl)-5-triisopropylsilanyloxypiperidin-4-yl]benzylsulphanyl}ethanol

A mixture of 2 mmol of 6-[(3R,4R,5S)-4-(4-chloromethylphenyl)-1-(toluene-4-sulphonyl)-5-triisopropylsilanyloxypiperidin-3-yloxymethyl]-4-(3-methoxypropyl)-3,4-dihydro-2H-benzo-[1,4]oxazine (Example 4c), 2 mmol of 2-mercaptoethanol and 3 mmol of potassium carbonate in 8 ml of N,N-dimethylformamide is stirred at room temperature for 4 hours. The reaction mixture is diluted with water and extracted with tert-butyl methyl ether (3×). The combined organic phases are washed with water, dried with sodium sulphate and evaporated. The title compound is identified on the basis of the Rf from the residue by flash chromatography (SiO₂ 60F).

### Example 52

### (R)-1-{(3S,4R,5R)-5-[4-(3-Methoxypropyl)-3,4-dihydro-2H-benzo[1,4]oxazin-6-ylmethoxy]-4-[4-(2-methylsulphanylethoxymethyl)phenyl]piperidin-3-yloxy}propan-2-ol

60.1 mg of lithium aluminium hydride are added to a solution of 263 mg of (R)-1-[(3S,4R,5R)-5-[4-(3-methoxypropyl)-3,4-dihydro-2H-benzo[1,4]oxazin-6-ylmethoxy]-4-[4-(2-methylsulphanylethoxymethyl)phenyl]-1-(toluene-4-sulphonyl)piperidin-3-yloxy]propan-2-ol in 4 ml of tetrahydrofuran. The suspension is heated at 50° C. for 26 hours, cooled to room temperature and, after cautious addition successively of 20 drops of water, 20 drops of 4N NaOH and 60 drops of water, stirred for 30 minutes. It is filtered through Hyflow and evaporated. The title compound is obtained from the residue by flash chromatography (SiO₂ 60F).

The starting materials are prepared as follows:

### a) (R)-1-[(3S,4R,5R)-5-[4-(3-Methoxypropyl)-3,4-dihydro-2H-benzo[1,4]oxazin-6-ylmethoxy]-4-[4-(2-methylsulphanylethoxymethyl)phenyl]-1-(toluene-4-sulphonyl)piperidin-3-yloxy]propan-2-ol

The title compound is obtained as a pale brown oil from (3S,4S,5R)-5-[4-(3-methoxypropyl)-3,4-dihydro-2H-benzo[1,4]oxazin-6-ylmethoxy]-4-[4-(2-methylsulphanylethoxymethyl)phenyl]-1-(toluene-4-sulphonyl)piperidin-3-ol (Example 50a) in analogy to Example 43a-b. Rf=0.25 (EtOAc/heptane 3:1); Rt=5.17 (gradient I).

The following compound is prepared in an analogous manner to the process described in Example 52:

### Example

### 67 (R)-1-{(3S,4R,5R)-4-[4-(2-Methoxyethylsulphanylmethyl)phenyl]-5-[4-(3-methoxypropyl)-3,4-dihydro-2H-benzo[1,4]oxazin-6-ylmethoxy]piperidin-3-yloxy}propan-2-ol

Starting from (3S,4S,5R)-4-[4-(2-methoxyethylsulphanylmethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 51a).

### Example 68

### Isopropyl{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}amine

The title compound is prepared from isopropyl[(3R,4R,5R)-4-[4-(2-methoxyethoxymethyl)phenyl]-5-[4-(3-methoxypropyl-3,4-dihydro-2H-benzo[1,4]oxazin-6-ylmethoxy]-1-(toluene-4-sulphonyl)piperidin-3-ylmethyl]amine in analogy to method L.

The starting materials are prepared as follows:

### a) Isopropyl[(3R,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl]amine

A solution of 0.50 mmol of (3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl toluene-4-sulphonate and 1.0 mmol of isopropylamine in 4 ml of 1-methylpyrrolidin-2-one (NMP) is stirred at 85° C. for 8 hours. The reaction mixture is cooled to room temperature, diluted with water and extracted with dichloromethane (3×). The combined organic phases are washed with brine, dried with sodium sulphate and evaporated. The title compound is identified on the basis of the Rf from the residue by flash chromatography (SiO₂ 60F).

### b) (3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl toluene-4-sulphonate

The title compound is identified on the basis of the Rf from 1 mmol of [(3S,4R,5R)-4-[4-(2-methoxyethoxymethyl)phenyl]-5-[4-(3-methoxypropyl)-3,4-dihydro-2H-benzo-[1,4]oxazin-6-ylmethoxy]-1-(toluene-4-sulphonyl)piperidin-3-yl]methanol in analogy to method H.

### c) [(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yl]methanol

The title compound is identified on the basis of the Rf from benzyl (3R,4R,5S)-3-hydroxy-4-(4-hydroxyphenyl)-5-triisopropylsilanyloxymethylpiperidine-1-carboxylate in analogy to the process described in Example 4a-j.

### d) Benzyl (3R,4R,5S-3-hydroxy-4-(4-hydroxyphenyl)-5-triisopropylsilanyloxymethylpiperidine-1-carboxylate

1.09 g of imidazole and 0.68 g of triisopropylchlorosilane are added to a solution of 1.76 g of benzyl (3R,4R,5S)-3-hydroxy-5-hydroxymethyl-4-(4-hydroxyphenyl)piperidine-1-carboxylate in 40 ml N,N-dimethylformamide at room temperature. After 16 hours, the reaction mixture is diluted with 1N HCl and extracted with tert-butyl methyl ether (3×). The combined organic phases are dried with sodium sulphate and evaporated. The title compound is identified on the basis of the Rf from the residue by flash chromatography (SiO₂ 60F).

### e) Benzyl (3R,4R,5S-3-hydroxy-5-hydroxymethyl-4-(4-hydroxyphenyl)piperidine-1-carboxylate

3.30 ml of benzyl chloroformate are slowly-added to a solution of 5.58 g (3R,4R,5S)-5-hydroxymethyl-4-(4-hydroxyphenyl)piperidin-3-ol hydrobromide [303043-56-3] in 100 ml of saturated sodium bicarbonate solution and 100 ml of ethyl acetate at 0° C., and the mixture is stirred for 5 hours. The reaction mixture is extracted with ethyl acetate/tetrahydrofuran (2×). The combined organic phases are evaporated and the title compound is identified on the basis of the Rf from the residue.

The following compounds are prepared in an analogous manner to the process described in Example 68:

### Examples

### 69 tert-Butyl{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}amine

### 70 {(3R,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}(2-methoxyethyl)amine

### 71 6-{(3R,4R,5S)-4-[4-(2-Methoxyethoxymethyl)phenyl]-5-morpholin-4-ylmethylpiperidin-3-yloxymethyl}-4-(3-methoxypropyl)-3,4-dihydro-2H-benzo[1,4]oxazine

### Example 72

### N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}acetamide

The title compound is prepared from N-[(3R,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl]acetamide in analogy to method L.

The starting materials are prepared as follows:

### a) N-[(3R,4R,5R)-4-[4-(2-Methoxyethoxymethyl)phenyl]-5-[4-(3-methoxy-propyl)-3,4-dihydro-2H-benzo[1,4]oxazin-6-ylmethoxy]-1-(toluene-4-sulphonyl)piperidin-3-ylmethyl]-acetamide

5 mmol of triethylamine and 1 mmol of propylphosphonic an hydride [68957-94-8, T3P] (50% in ethyl acetate) are successively added to a solution of 1 mmol of C-[(3R,4R,5R)-4-[4-(2-methoxyethoxymethyl)phenyl]-5-[4-(3-methoxypropyl)-3,4-dihydro-2H-benzo-[1,4]oxazin-6-ylmethoxy]-1-(toluene-47sulphonyl)piperidin-3-yl]methylamine and 1 mmol of acetic acid in 20 ml of dichloromethane at room temperature. After 12 hours, the reaction mixture is diluted with dichloromethane and washed successively with 1N HCl and brine, dried with sodium sulphate and evaporated. The title compound is identified on the basis of the Rf from the residue by flash chromatography (SiO₂ 60F).

### b) C-[(3R,4R,5R)-4-[4-(2-Methoxyethoxymethyl)phenyl]-5-[4-(3-methoxypropyl)-3,4-d hydro-2H-benzo[1,4]oxazin-6-ylmethoxy]-1-(toluene-4-sulphonyl)piperidin-3-yl]-methylamine

A solution of 0.5 mmol of 6-[(3R,4R,5R)-5-azidomethyl-4-[4-(2-methoxyethoxymethyl)phenyl]-1-(toluene-4-sulphonyl)piperidin-3-yloxymethyl]-4-(3-methoxypropyl)-3,4-dihydro-2H-benzo[1,4]oxazine in. 15 ml of tetrahydrofuran is hydrogenated in the presence of 50 mg of 10% Pd/C (moist) at room temperature for 6 hours. The reaction mixture is clarified by filtration and the filtrate is evaporated. The title compound is identified on the basis of the Rf from the residue by flash chromatography (SiO₂ 60F).

### c) 6-[(3R,4R,5R)-5-Azidomethyl-4-[4-(2-methoxyethoxymethyl)phenyl]-1-(toluene-4-sulphonyl)piperidin-3-yloxymethyl]-4-(3-methoxypropyl)-3,4-dihydro-2H-benzo-[1,4]oxazine

A solution of 0.50 mmol of (3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl toluene-4-sulphonate (Example 68b) and 0.75 mmol of sodium azide in 5 ml of N,N-dimethylformamide is stirred at room temperature for 24 hours. The reaction mixture is diluted with water and extracted with tert-butyl methyl ether (3×). The combined organic phases are dried with sodium sulphate and evaporated. The title compound is identified on the basis of the Rf from the residue by flash chromatography (SiO₂ 60F).

The following compounds are prepared in an analogous manner to the process described in Example 72:

### Examples

### 101 N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}pentanamide

### 103 N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}-2-(tetrahydropyran-4-yl)acetamide

Using (tetrahydropyran-4-yl)acetic acid [85064-61-5]

### 104 N-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}tetrahydropyran-4-carboxamide

Using tetrahydropyran-4-carboxylic acid [5337-03-1]

### 105 2-Cyclopentyl-N-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}acetamide

Using cyclopentylacetic acid [1123-00-8]

### 106 N-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}-(meso-1S,5R,6R)-3-oxabicyclo[3.1.0]hexane-6-carboxamide

Using (meso-1S,5R,6R)-3-oxabicyclo[3.1.0]hexane-6-carboxylic acid [55780-88-6]

### 107 N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}-2-(meso-1R,5S,6S)-3-oxabicyclo[3.1.0]hex-6-ylacetamide

Using (meso-1R,5S,6S)-(3-oxabicyclo[3.1.0]hex-6-yl)acetic acid

The starting materials are prepared as follows:

### a) (meso-1R,5S,6S)-(3-Oxabicyclo[3.1.0]hex-6-yl)acetic acid

3 mmol of triethylamine and 0.5 mmol of silver trifluoroacetate are added to a solution of 1 mmol of 1-diazo-3-(meso-1R,5S,6S)-3-oxabicyclo[3.1.0]hex-6-ylpropan-2-one in 70 ml of tetrahydrofuran/water 10:1 at −15° C. The reaction mixture is warmed to room temperature and stirred at room temperature for 2 hours. It is diluted with tert-butyl methyl ether, washed with 1M HCl and brine, dried with sodium sulphate and evaporated. The title compound is identified on the basis of the Rf from the residue by flash chromatography (SiO₂ 60F).

### b) 1-Diazo-3-(meso-1R,5S,6S)-3-oxabicyclo[3.1.0]hex-6-yl-Propan-2-one

1.2 mmol of triethylamine and 1 mmol of ethyl chloroformate are added to a solution of 1 mmol of (meso-1S,5R,6R)-3-oxabicyclo[3.1.0]hexane-6-carboxylic acid [55780-88-6] in 60 ml of tetrahydrofuran at −15° C. The reaction mixture is warmed to −5° C. and stirred at this temperature for 1 hour. It is cooled to −30° C., and 2.5 mmol of a diazomethane solution in ether are added and the mixture is stirred overnight. It is diluted with tert-butyl methyl ether, washed with saturated aqueous sodium bicarbonate solution and brine, dried with sodium sulphate and evaporated. The title compound is identified on the basis of the Rf from the residue by flash chromatography (SiO₂ 60F).

### 108 N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}-4-methoxy-cyclohexanecarboxamide

using 4-methoxycyclohexanecarboxylic acid [99183-14-9]

### 125 N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}cyclopentane-carboxamide

Using cyclopentancarboxylic acid [340045-1]

### 126 2-Ethyl-N-{(3S,4R,5R)-4-[4-(2-methoxyethoxymethyl)phenyl]-5-[4-(3-methoxypropyl)-3,4-dihydro-2H-benzo[1,4]oxazin-6-ylmethoxy]piperidin-3-ylmethyl}butyramide

Using 2-ethylbutyric acid [88-09-5]

### Example 82

### (S)-4-{(3S,4S,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}butan-2-ol

The title compound is prepared from (S)-4-[(3S,4S,5R)-4-[4-(2-methoxyethoxymethyl)-phenyl]-5-[4-(3-methoxypropyl)-3,4-dihydro-2H-benzo[1,4]oxazin-6-ylmethoxy]-1-(toluene-4-sulphonyl)piperidin-3-yloxy]butan-2-ol in analogy to method L.

The starting materials are prepared as follows:

### a) (S)-4-[(3S,4S,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]butan-2-ol

The title compound is obtained as a colourless wax from 1.04 g of 6-[(3R,4S,5S)-4-[4-(2-methoxyethoxymethyl)phenyl]-1-(toluene-4-sulphonyl)-5-((S)-3-triisopropylsilanyloxybutoxy)-piperidin-3-yloxymethyl]-4-(3-methoxypropyl)-3,4-dihydro-2H-benzo[1,4]oxazine in analogy to method J. Rf=0.07 (EtOAc/heptane 3:1).

### b) 6-[(3R,4S,5S)-4-[4-(2-Methoxyethoxymethyl)phenyl]-1-(toluene-4-sulphonyl)-5-((S)-3-triisopropylsilanyloxybutoxy)piperidin-3-yloxymethyl]-4-(3-methoxypropyl)-3,4-dihydro-2H-benzo[1,4]oxazine

140 mg of sodium hydride (60% dispersion in oil) are added to a solution of 1.04 g of (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22b) in 8 ml of DMF at 0° C., and the mixture is stirred for 1 hour. It is then cooled to −5° C., and 1.27 g of (S)-triisopropylsilanyloxybutyl toluene-4-sulphonate are added. The reaction mixture is stirred at 60° C. for 3 hours and then cooled to room temperature. It is subsequently diluted with tert-butyl methyl ether and poured into ice-water. The resulting mixture is extracted three times with tert-butyl methyl ether. The combined organic phases are washed with brine, dried with sodium sulphate and evaporated. The title compound is obtained as a colourless oil from the residue by flash chromatography (SiO₂ 60F). Rf=0.28 (EtOAc/heptane 1:1).

### c) (S)-3-triisopropylsilanyloxybutyl toluene-4-sulphonate

7.33 ml of lutidine are added to a solution of 10 g of (S)-3-hydroxybutyl toluene-4-sulphonate [82614-84] in 100 ml of dichloromethane at 0° C. 12.49 ml of triisopropylsilyl trifluoromethanesulphonate are added dropwise, and the mixture is stirred at 0° C. for 1 hour. It is quenched with 0.5M HCl and extracted with dichloromethane (2×). The combined organic phases are washed with brine, dried with sodium sulphate and evaporated. The title compound is obtained as a colourless liquid from the residue by flash chromatography (SiO₂ 60F). Rf=0.72 (EtOAc/heptane 1:1); Rt=6.64 (gradient I).

The following compounds are prepared in an analogous manner to the process described in Example 82:

### Examples

### 83 (R)-4-{(3S,4S,5R)-4-[4-(2-Methoxyethoxymethyl)phenyl]-5-[4-(3-methoxypropyl-3,4-dihydro-2H-benzo[1,4]oxazin-6-ylmethoxy]piperidin-3-yloxy}butan-2-ol

Using tert-butyl ((R)-3-iodo-1-methylpropoxy)dimethylsilane [109715-47-1]

### 84 (R-4-{(3S,4S,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 48a) using tert-butyl ((R)-3-iodo-1-methylpropoxy)dimethylsilane [109715-47-1]

### 85 (S)-4-{(3S,4S,5R)-4-[4-(3-Methoxypropoxymethyl)phenyl]-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-[4-(3-methoxypropoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 48a).

### 88 (S)-4-{(3S,4S,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b).

### 89 (R)-4{(3S,4S,5R)-4-(4-Cyclopropylmethoxymethylphenyl)-5-[4-(3-methoxypropyl)-3,4-dihydro-2H-benzo[1,4]oxazin-6-ylmethoxy]piperidin-3-yloxy}butan-2-ol

Starting from (3S,4S,5R)-4-(4-cyclopropylmethoxymethylphenyl)-5-[4-(3-methoxypropyl)-3,4-dihydro-2H-benzo[1,4]oxazin-6-ylmethoxy]-1-(toluene-4-sulphonyl)piperidin-3-ol (Example 15b) using tert-butyl ((R)-3-iodo-1-methylpropoxy)dimethylsilane [109715-47-1].

### 95 (R)-4-{(3S,4S,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}-butan-2-ol

Starting from (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ol (Example 5a) using tert-butyl((R)-3-iodo-1-methylpropoxy)dimethylsilane [109715-47-1].

### 97 (S)-4-{(3S,4S,5R)-4-[4-((S)-3-Methoxy-2-methylpropoxymethyl)phenyl]-5-[4-(3-methoxypropyl)-3,4-dihydro-2H-benzo[1,4]oxazin-6-ylmethoxy]piperidin-3-yloxy}-butan-2-ol

Starting from (3S,4S,5R)-4-[4-((S)-3-methoxy-2-methylpropoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ol (Example 5a).

### Example 100

### N-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-ylmethyl}acetamide

The title compound is prepared by the process described in Example 68 and 72

The following compound is prepared in an analogous manner to the process described in Example 100:

### Example

### 102 N-{(3S,4R,5R)-4-(4-Cyclopropylmethoxymethylphenyl)-5-[4-(3-methoxypropyl)-3,4-dihydro-2H-benzo[1,4]oxazin-6-ylmethoxy]piperidin-3-ylmethyl}pentanamide

### Example 109

### N-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-ylmethyl}morpholine-4-carboxamide

The title compound is prepared from N-[(3R,4R,5R)-4-[4-2-methoxyethoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ylmethyl]morpholine-4-carboxamide in analogy to method L.

The starting material is prepared as follows:

### a) N-[(3R,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl]-morpholine-4-carboxamide

3 mmol of triethylamine are added to a solution of 1 mmol of C-[(3R,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yl]methylamine (Example 72b) and 1.1 mmol of morpholine-4-carbonyl chloride [1515940-7] in 20 ml of dichloromethane at 0° C. After 1.5 hours, the reaction mixture is poured into 1M sodium bicarbonate solution and extracted with tert-butyl methyl ether (3×), and the combined organic phases are washed with brine, dried with sodium sulphate and evaporated. The title compound is obtained as a yellowish oil from the residue by flash chromatography (SiO₂ 60F). Rf=0.29 (dichloromethane/methanol/25% conc. ammonia=200:20:1); Rt=4.58 (gradient I).

### Example 114

### (R)-1-((3S,4S,5R)-4-[4-(2-Methoxyethoxymethyl)phenyl]-5-{2-[4-(3-methoxypropyl)-3,4-dihydro-2H-benzo[1,4]oxazin-6-yl]ethyl}piperidin-3-yloxy)propan-2-ol

The title compound is prepared from 0.420 g of (R)-1-[(3S,4S,5R)-4-[4-(2-methoxyethoxymethyl)phenyl]-5-{2-[4-(3-methoxypropyl)-3,4-dihydro-2H-benzo[1,4]oxazin-6-yl]ethyl}-1-(toluene-4-sulphonyl)piperidin-3-yloxy]propan-2-ol in analogy to method L.

The starting materials are prepared as follows:

### a) (R)-1-[(3S,4S,5R)-4-[4-(2-Methoxyethoxymethyl)phenyl]-5-{2-[4-(3-methoxypropyl)-3,4-dihydro-2H-benzo[1,4]oxazin-6-yl]ethyl}-1-(toluene-4-sulphonyl)piperidin-3-yloxy]propan-2-ol

0.10 g of sodium borohydride is added to a solution of 0.67 g of 6-{2-[(3R,4S,5S)-4-[4-(2-methoxyethoxymethyl)phenyl]-5-((R)-1-oxiranylmethoxy)-1-(toluene-4-sulphonyl)piperidin-3-yl]ethyl}-4-(3-methoxypropyl)-3,4-dihydro-2H-benzo[1,4]oxazine in 10 ml of ethanol and 0.75 ml of tetrahydrofuran, and the mixture is stirred at 45° C. for 18 hours. The reaction mixture is poured into 1M ammonium chloride (50 ml) and extracted with tert-butyl methyl ether (2×50 ml). The combined organic phases are washed with brine (50 ml), dried with sodium sulphate and evaporated. The title compound is obtained from the residue by flash chromatography (SiO₂ 60F) and identified on the basis of the Rf.

### b) 6-{2-[(3R,4S,5S)-4-[4-(2-Methoxyethoxymethyl)phenyl]-5-((R)-1-oxiranylmethoxy)-1-(toluene-4-sulphonyl)piperidin-3-yl]ethyl}-4-(3-methoxypropyl)-3,4-dihydro-2H-benzo[1,4]oxazine

1.20 g of (3S,4S,5R)-4-[4-(2-methoxyethoxymethyl)phenyl]-5-{2-[4-(3-methoxypropyl)-3,4-dihydro-2H-benzo[1,4]oxazin-6-yl]ethyl}-1-(toluene-4-sulphonyl)piperidin-3-ol are reacted in analogy to Example 31a. The title compound is identified on the basis of the Rf.

### c) (3S,4S,5R)-4-[4-(2-Methoxyethoxymethyl)phenyl]-5-{2-[4-(3-methoxypropyl)-3,4-dihydro-2H-benzo[1,4]oxazin-6-yl]ethyl}-1-(toluene-4-sulphonyl)piperidin-3-ol

2.0 g of 6-(2-[(3R,4S,5S)-4-[4-(2-methoxyethoxymethyl)phenyl]-1-(toluene-4-sulphonyl)-5-triisopropylsilanyloxypiperidin-3-yl]ethyl)-4-(3-methoxypropyl)-3,4-dihydro-2H-benzo[1,4]oxazine are reacted in analogy to method J. The title compound is identified on the basis of the Rf.

### d) 6-{2-[(3R,4S,5S)-4-[4-(2-methoxyethoxymethyl)phenyl]-1-(toluene-4-sulphonyl)-5-triisopropylsilanyloxypiperidin-3-yl]ethyl}-4-(3-methoxypropyl)-3,4-dihydro-2H-benzo[1,4]oxazine

0.747 ml of 2-bromoethyl methyl ether and 1.48 g of tetrabutylammonium iodide are successively added to a stirred solution of 3.0 g of {4-[(3R,4S,5S)-3-{2-[4-(3-methoxypropyl)-3,4-dihydro-2H-benzo[1,4]oxazin-6-yl]ethyl}-1-(toluene-4-sulphonyl)-5-triisopropylsilanyloxy-piperidin-4-yl]phenyl}methanol in 15 ml of N,N-dimethylformamide. The mixture is cooled to −5° C. and, after addition of 0.316 g of sodium hydride dispersion (60% in oil), stirred at room temperature for 24 hours. The reaction mixture is poured into ice-water (60 ml) and extracted with dichloromethane (3×60 ml). The combined organic phases are washed with water (2×150 ml) and brine (150 ml), dried with sodium sulphate and evaporated. The title compound is obtained from the residue by flash chromatography (SiO₂ 60F) and identified on the basis of the Rf.

### e) {4-[(3R,4S,5S)-3-{2-[4-(3-Methoxypropyl)-3,4-dihydro-2H-benzo[1,4]oxazin-6-yl]ethyl}-1-(toluene-4-sulphonyl)-5-triisopropylsilanyloxypiperidin-4-yl]phenyl}methanol

4.50 g of 4-[(3R,4S,5S)-3-{2-[4-(3-methoxypropyl-3-oxo-3,4-dihydro-2H-benzo[1,4]oxazin-6-yl]ethyl}-1-(toluene-4-sulphonyl)-5-triisopropylsilanyloxypiperidin-4-yl]benzoic acid are reacted in analogy to method K. The title compound is identified on the basis of the Rf.

### f) 4-[(3R,4S,5S)-3-{2-[4-(3-Methoxypropyl)-3-oxo-3,4-dihydro-2H-benzo[1,4]oxazin-6-yl]-ethyl}-(toluene-4-sulphonyl)-5-triisopropylsilanyloxypiperidin-4-yl]benzoic acid

6.0 g of methyl 4-[(3R,4S,5S)-3{2-[4-(3-methoxypropyl)-3-oxo-3,4-dihydro-2H-benzo[1,4]oxazin-6-yl]ethyl}-(toluene-4-sulphonyl)-5-triisopropylsilanyloxypiperidin-4-yl]benzoate are reacted in analogy to Example 4g. The title compound is identified on the basis of the Rf.

### g) Methyl 4-[(3R,4S,5S)-3-{2-[4-(3-Methoxypropyl)-3-oxo-3,4-dihydro-2H-benzo[1,4]oxazin-6-yl]ethyl}-1-(toluene-4-sulphonyl)-5-triisopropylsilanyloxypiperidin-4-yl]benzoate

A solution of 7.25 g of methyl 4-[(3R,4S,5S)-3-{2-[4-(3-methoxypropyl)-3-oxo-3,4-dihydro-2H-benzo[1,4]oxazin-6-yl]vinyl}-1-(toluene-4-sulphonyl)-5-triisopropylsilanyloxypiperidin-4-yl]benzoate in 80 ml of ethanol is hydrogenated in the presence of 0.80 g of Pd/C 10% at room temperature for 2 hours. The reaction mixture is clarified by filtration and the filtrate is evaporated. The title compound is obtained from the residue by flash chromatography (SiO₂ 60F) and identified on the basis of the Rf.

### h) Methyl 4-[(3R,4S,5S-3-{2-[4-3-methoxypropyl)-3-oxo-3,4-dihydro-2H-benzo[1,4]oxazin-6-yl]vinyl}-1-(toluene-4-sulphonyl)-5-triisopropylsilanyloxypiperidin-4-yl]benzoate

10.0 ml of n-butyllithium (1.6M in hexane) are added to a stirred suspension of 11.90 g of [4-(3-methoxypropyl)-3-oxo-3,4-dihydro-2H-benzo[1,4]oxazin-6-ylmethyl]triphenyl-phosphonium chloride (Example 114s) and 100 ml of tetrahydrofuran at 0° C., and the mixture is stirred at room temperature for 1 hour. A solution of 8.0 g of methyl 4-[(3R,4S,5S)-3-formyl-1-(toluene-4-sulphonyl)-5-triisopropylsilanyloxypiperidin-4-yl]benzoate in 50 ml of tetrahydrofuran is added to the reaction mixture over the course of 10 minutes, and the mixture is then stirred at room temperature for 4 hours. The reaction mixture is poured into 1M ammonium chloride solution (250 ml) and extracted with tert-butyl methyl ether (2×250 ml). The combined organic phases are washed with brine (250 ml), dried with sodium sulphate and evaporated. The title compound is obtained from the residue by flash chromatography (SiO₂ 60F) and identified on the basis of the Rf.

### i) Methyl 4-[(3R,4S,5S)-3-formyl-1-(toluene-4-sulphonyl)-5-triisopropylsilanyloxypiperidin-4-yl]benzoate

8.10 g of 3A molecular sieves and 2.54 g of 4-methylmorpholine N-oxide are added to a stirred solution of 8.10 g of methyl 4-[(3R,4S,5S)-3-hydroxymethyl-1-(toluene-4-sulphonyl)-5-triisopropylsilanyloxypiperidin-4-yl]benzoate in 160 ml of dichloromethane, and the mixture is stirred at room temperature for 10 minutes. 0.247 g of tetra-N-propylammonium perruthenate(VII) is added to the reaction mixture, which is then stirred at room temperature for 20 minutes. The resulting mixture is clarified by filtration and the filtrate is washed successively with 2M sodium sulphite (80 ml), brine (80 ml) and 2M copper(II) sulphate (80 ml). The organic phase is dried with sodium sulphate and evaporated. The crude title compound is identified on the basis of the Rf.

### j) Methyl 4-[(3R,4S,5S-3-hydroxymethyl-1-(toluene-4-sulphonyl)-5-triisopropylsilanyloxypiperidin-4-yl]benzoate

1.90 g of p-toluenesulphonic acid are added to a stirred solution of 8.18 g of methyl 4-[(3S,4S,5R)-1-(toluene-4-sulphonyl)-3-triisopropylsilanyloxy-5-trityloxymethylpiperidin-4-yl]benzoate and 100 ml of methanol/tetrahydrofuran (1:1) at 0° C., and then the mixture is stirred for 20 hours at room temperature. The reaction mixture is poured into ice-cold 1M NaOH (250 ml) and extracted with tert-butyl methyl ether (2×250 ml). The combined organic phases are washed with brine (250 ml), dried with sodium sulphate and evaporated. The title compound is obtained from the residue by flash chromatography (SiO₂ 60F) and identified by means of the Rf.

### k) Methyl 4-[(3S,4S,5R)-1-(toluene-4-sulphonyl)-3-triisopropylsilanyloxy-5-trityloxymethylpiperidin-4-yl]benzoate

4.85 ml of triisopropyl trifluoromethanesulphonate are added to a solution of 9.92 g of methyl 4-[(3S,4S,5R)-3-hydroxy-1-(toluene-4-sulphonyl)-5-trityloxymethylpiperidin-4-yl]benzoate, 2.61 ml of 2,6-lutidine in 150 ml of dichloromethane over the course of 10 minutes at 0° C., and the mixture is stirred for 3 hours. The reaction mixture is poured into ice-water (250 ml) and extracted with tert-butyl methyl ether (2×250 ml). The organic phases are washed with brine (250 ml), dried with sodium sulphate and evaporated. The title compound is obtained as a yellow oil from the residue by flash chromatography (SiO₂ 60F) and is identified on the basis of the Rf.

### l) Methyl 4-[(3S,4S,5R)-3-hydroxy-1-toluene-4-sulphonyl)-5-trityloxymethylpiperidin-4-yl]benzoate

A mixture of 0.29 g of trityl chloride, 0.43 g of methyl 4-[(3S,4S,5R)-3-hydroxy-5-hydroxymethyl-1-(toluene-4-sulphonyl)-piperidin-4-yl]-benzoate and 0.006 g of 4-dimethylaminopyridine is diluted with 2 ml of pyridine and then the reaction mixture is stirred at 70° C. for 12 hours. The reaction mixture is evaporated, diluted with 1:1 ice/1N aqueous hydrochloric acid and extracted twice with tert-butyl methyl ether. The combined organic phases are washed with 1M aqueous sodium bicarbonate solution and brine, dried with sodium sulphate and evaporated. The title compound is obtained as a white foam from the residue by flash chromatography (SiO₂ 60F) and is identified on the basis of the Rf.

### m) Methyl 4-[(3S,4S,5R-3-Hydroxy-5-hydroxymethyl-1-(toluene-4-sulphonyl)-piperidin-4-yl]-benzoate

14.62 g of 4-[(3S,4S,5R)-3-hydroxy-5-hydroxymethyl-1-(toluene-4-sulphonyl)piperidin-4-yl]phenyl trifluoromethanesulphonate are reacted in analogy to Example 4i. The title compound is identified on the basis of the Rf.

### n) 4-[(3S,4S,5R)-3-Hydroxy-5-hydroxymethyl-1-(toluene-4-sulphonyl)piperidin-4-yl]phenyl trifluoromethanesulphonate

11.30 g of (3S,4S,5R)-5-hydroxymethyl-4-(4-hydroxyphenyl)-1-(toluene-4-sulphonyl)piperidin-3-ol are reacted in analogy to Example 4j. The title compound is identified on the basis of the Rf.

### o) (3S,4S,5R)-5-Hydroxymethyl-4-(4-hydroxyphenyl)-1-(toluene-4-sulphonyl)piperidin-3-ol

8.90 g of (3S,4S,5R)-5-hydroxymethyl (4-hydroxyphenyl)piperidin-3-ol are reacted in analogy to Example 4d. The title compound is identified on the basis of the Rf.

### p) (3S,4S,5R)-5-Hydroxymethyl-4-(4-hydroxyphenyl)piperidin-3-ol

17.2 g of (3S,4S,5R)-1-benzyl-5-hydroxymethyl-4-(4-hydroxyphenyl)piperidin-3-ol hydrobromide are reacted in analogy to method B. The title compound is identified on the basis of the Rf.

### q) (3S,4S,5R)—-Benzyl-5-hydroxymethyl-4-(4-hydroxyphenyl)Piperidin-3-ol hydrobromide

160 ml of 1M boron tribromide (in dichloromethane) are added over the course of 15 minutes to a solution of 22.8 g of (3S,4S,5R)-1-benzyl-4-(4-methoxyphenyl)-5-trityloxymethylpiperidin-3-ol and 900 ml of dichloromethane at 0° C., and the mixture is stirred for 1 hour. The mixture is cooled to −15° C., and the crystals are filtered off with suction. The material on the filter is taken up in 900 ml of methanol and then evaporated to dryness in a rotary evaporator. The title compound is obtained from the residue and identified on the basis of the Rf.

### r) (3S,4S,5R)-1-Benzyl-4-(4-methoxyphenyl)-5-trityloxymethylpiperidin-3-ol

1.46 g of (D>(−)-mandelic acid are added to a solution of 9.12 g of (R,S)-(3S,4S,5R)-1-benzyl-4-(4-methoxyphenyl)-5-trityloxymethylpiperidin-3-ol [188879-88-1] in 110 ml of tetrahydrofuran at 60° C. (oil bath temperature). 110 ml of n-hexane are slowly added dropwise at 60° C. The mixture is slowly cooled to room temperature over the course of 3 hours and, after a brief treatment in an ultrasonic bath, then cooled at 0° C. for 2 hours. The precipitate is filtered off and washed with tetrahydrofuran/n-hexane 1:3 (2×20 ml). The salt is dissolved in ethyl acetate and washed with saturated aqueous sodium carbonate solution (2×). The combined organic phases are washed with brine, dried with sodium sulphate and evaporated. The title compound is obtained from the residue. HPLC; Rt=24.10 (chiralpak AD 0.46×25 cm daicel; 95% hexane/5% isopropanol flow. 0.7 ml/minute (total 60 minutes).

### s) [4-(3-Methoxypropyl)-3-oxo-3,4-dihydro-2H-benzo[1,4]oxazin-6-ylmethyl]triphenylphosphonium chloride

10.3 g of triphenylphosphine are added to a stirred solution of 10.09 of 6-chloromethyl-4-(3-methoxypropyl)-4H-benzo[1,4]oxazin-3-one (Example 4o) in 100 ml of xylene, and the mixture is refluxed for 18 hours. The reaction mixture is cooled to room temperature, and the solid is filtered off with suction. The title compound is identified on the basis of the Rf.

### Example 115

### (3S,4S,5R)-4-[4-((1S,2S)-2-Methoxycyclopropylmethoxymethyl)phenyl]-5-[4-(3-methoxypropyl)-3,4-dihydro-2H-benzo[1,4]oxazin-6-ylmethoxy]piperidin-3-ol

The title compound is prepared from (3S,4S., 5R)-4-[4-((1S,2S-2-methoxycyclopropylmethoxymethyl)phenyl]-5-[4-3-methoxypropyl)-3,4-dihydro-2H-benzo[1,4]oxazin-6-ylmethoxy]-1-(toluene-4-sulphonyl)piperidin-3-l in analogy to method L and identified on the basis of the Rf.

The starting materials are prepared as follows:

### a) (3S,4S,5R)-4-[4-((1S,2S)-2-Methoxycyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol

The title compound is prepared from 6-[(3R,4R,5S)-4-[4-((1S,2S-2-methoxycyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine in analogy to method J and identified on the basis of the Rf.

### b) 6-[(3R,4R,5S)-4-[4-((1S,2S)-2-Methoxycyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

The title compound is prepared starting from 6-[(3R,4R,5S)-4-(4-chloromethylphenyl) 1-(toluene-4-sulphonyl)-5-triisopropylsilanyloxypiperidin-3-yloxymethyl]-4-(3-methoxypropyl)-3,4-dihydro-2H-benzo[1,4]oxazine (Example 4c) and ((1R,2S)-2-methoxycyclopropyl)-methanol in analogy to Example 4b and identified on the basis of the Rf.

### c) ((1R,2S)-2-Methoxycyclopropyl)methanol

0.560 g of lithium borohydride is added to a solution of 4.410 g of (R)-4-benzyl-3-((1S,2S)-2-methoxycyclopropanecarbonyl)oxazolidin-2-one in 40 ml of tetrahydrofuran and 1 ml of methanol at 0° C. After the addition is complete, the reaction mixture is stirred for 3 hours at 0° C., and then phosphate buffer is added with pH 7. The mixture is extracted with ethyl acetate, and the combined organic extracts are washed with brine, dried with sodium sulphate and evaporated. The title compound is obtained from the residue by flash chromatography (SiO₂ 60F) and identified on the basis of the Rf.

### d) (R)-4-Benzyl-3-((1S,2S-2-methoxycyclopropanecarbonyl)oxazolidin-2-one

and

### (R)-4-Benzyl-3-((1R,2R-2-methoxycyclopropanecarbonyl)oxazolidin-2-one

A solution of 2.000 g of (R)benzyl-2-oxazolidinone in 11 ml of dry tetrahydrofuran is cooled to −75° C. 7.10 ml of n-butyllithium solution (1.6M in hexane) are added dropwise to the solution at −75-−70° C. After the addition is complete, the reaction mixture is stirred at −75° C. for 10 minutes and then a solution of 1.346 g of trans-2-methoxycyclopropanecarbonyl chloride in 10 ml of tetrahydrofuran is added. The reaction solution is warmed to room temperature and saturated aqueous ammonium chloride solution is added, and the mixture is extracted with tert-butyl methyl ether. The combined organic extracts are washed with brine, dried with sodium sulphate and evaporated. The title compounds are identified on the basis of the Rf from the residue by flash chromatography (SiO₂ 60F).

### e) trans-2-Methoxycyclopropanecarbonyl chloride

1.01 ml of oxalyl chloride are added to a solution of 1.160 g of trans-2-methoxycyclopropanecarboxylic acid [60212-42-2] in 10 ml of dichloromethane at 0° C. One drop of N,N-dimethyl-formamide is added, and the reaction solution is stirred at 0° C. for one hour and then evaporated. The residue is employed without further purification in the next stage.

### Example 116

### (3S,4S,5R)-4-[4-((1S,2S)-2-Methoxymethylcyclopropylmethoxymethyl)phenyl]-5-[4-(3-methoxypropyl)-3,4-dihydro-2H-benzo[1,4]oxazin-6-ylmethoxy]piperidin-3-ol

The title compound is prepared from (3S,4S,5R)-4-[4-((1S,2S)-2-methoxymethylcyclopropylmethoxymethyl)phenyl]-5-[4-(3-methoxypropyl-3,4-dihydro-2H-benzo[1,4]oxazin-4-ylmethoxy]-1-(toluene-4-sulphonyl)piperidin-3-l in analogy to method L, and identified on the basis of the Rf.

The starting materials are prepared as follows:

### a) (3S,4S,5R)-4-[4-((1S,2S)-2-Methoxymethylcyclopropylmethoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ol

The title compound is obtained from 6-[(3R,4R,5S)-4-[4-2-((1S,2S)-2-methoxymethylcyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine in analogy to method J, and identified on the basis of the Rf.

### b) 6-[(3R,4R,5S)-4-[4-2-((1S,2S)-2-Methoxymethylcyclopropylmethoxymethyl)phenyl]-1-(toluene-4-sulphonyl)-5-triisopropylsilanyloxypiperidin-3-yloxymethyl]-4-(3-methoxypropyl)-3,4-dihydro-2H-benzo[1,4]oxazine

0.032 g of sodium hydride (60% dispersion in paraffin) is taken up in 5 ml of N,N-dimethylformamide, and the suspension is cooled to −10° C. A solution of 0.0406 g of ((1S,2S)-2-methoxymethylcyclopropyl)methanol in 2 ml of N,N-dimethylformamide is added dropwise over the course of 5 minutes, and the reaction mixture is then stirred at −10° C. for 10 minutes.

A solution of 0.400g of 6-[(3R,4R,5S)-4-(4-chloromethylphenyl)-1-(toluene-4-sulphonyl)-5-triisopropylsilanyioxypiperidin-3-yloxymethyl]-4-(3-methoxypropyl)-3,4-dihydro-2H-benzo[1,4]oxazine (Example 4c) in 3 ml of N,N-dimethylformamide is added dropwise, and the reaction mixture is stirred at room temperature for 16 hours. The reaction mixture is poured into water, and the aqueous phase is extracted with tert-butyl methyl ether. The combined organic extracts are washed with brine, dried with sodium sulphate and evaporated. The title compound is obtained as a colourless oil from the residue by flash chromatography (SiO₂ 60F). Rf=0.31 (EtOAc/heptane 1:2).

### c) ((1S,2S-2-Methoxymethylcyclopropyl)methanol

A suspension of 0.083 g of lithiumaluminium hydride in 5 ml of diethyl ether is cooled to 0° C.

A solution of 0.220 g of ethyl (1S,2S)-2-methoxymethylcyclopropanecarboxylate in 5 ml of diethyl ether is added dropwise at 0° C., and the reaction mixture is stirred at this temperature for 2 hours. Water, 4M sodium hydroxide solution and again water are successively added to the reaction mixture, the resulting solid is filtered off through Hyflo, the filter cake is washed with diethyl ether, and the filtrate is evaporated. The title compound is obtained as a colourless liquid and employed without further purification in the next stage.

### d) Ethyl (1S,2S)-2-methoxymethylcyclopropanecarboxylate

4.60 ml of triethyl phosphonoacetate are added dropwise over 5 minutes to a suspension of 0.940 g of sodium hydride (60% dispersion in oil) in 10 ml of toluene. The reaction mixture is stirred for 10 minutes and then 1.01 g of (R-(−)-glycidyl methyl ether are added, and the mixture is heated to reflux for 16 hours. The reaction mixture is cooled to room temperature and diluted with tert-butyl methyl ether, and saturated aqueous ammonium chloride solution is added. The phases are separated, the aqueous phase is extracted with tert-butyl methyl ether, and the combined organic phases are washed with brine, dried with sodium sulphate and evaporated. The title compound is obtained as a yellowish oil from the residue by flash chromatography (SiO₂ 60F). Rf=0.10 (diethyl ether/hexane 1:4).

### Example 117

### (R)-1-{(3S,4R,5R)-4-[4-((1S,2S)-2-Methoxycyclopropylmethoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is obtained starting from (3S,4S,5R)-4-[4-((1S,2S)-2-methoxycyclopropylmethoxymethyl)phenyl]-5-[4-3-methoxypropyl)-3,4-dihydro-2H-benzo[1,4]oxazin-6-ylmethoxy]-1-(toluene-4-sulphonyl)piperidin-3-ol (Example 115a) in analogy to the process described in Example 31. Deprotection of the protective group on the nitrogen (last stage of the synthesis) is carried out in analogy to method L. The title compound is identified on the basis of the Rf.

### Example 118

### (R)-1-{(3S,4R,5R)-4-[4-((1S,2S)-Methoxymethylcyclopropylmethoxymethyl)phenyl]-5-[4-(3-methoxypropyl)-3,4-dihydro-2H-benzo[1,4]oxazin-6-ylmethoxy]piperidin-3-yloxy}propan-2-ol

The title compound is obtained starting from (3S,4S,5R)-4-[4-((1S,2S)-2-methoxymethylcyclopropylmethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 116a) in analogy to the process described in Example 31. Deprotection of the protective group on the nitrogen (last stage of the synthesis) is carried out in analogy to method L. The title compound is identified on the basis of the Rf.

### Example 119

### (3S,4S,5R)-4-[4-(2-Methoxyethoxymethyl)phenyl]-5-[4-(3-methoxypropyl)-2,2-dimethyl-2H-chromen-6-ylmethoxy]piperidin-3-ol

The title compound is prepared from (3S,4S,5R)-4-[4-(2-methoxyethoxymethyl)phenyl]-5-[4-(3-methoxypropyl)-2,2-dimethyl-2H-chromen-6-ylmethoxy]-1-(toluene-4-sulphonyl)-piperidin-3-ol in analogy to method L and identified on the basis of the Rf.

The starting materials are prepared as follows:

### a) (3S,4S,5R)-4-[4-(2-Methoxyethoxymethyl)phenyl]-5-[4-(3-methoxypropyl)-2,2-dimethyl-2H-chromen-6-ylmethoxy]-1-(toluene-4-sulphonyl)piperidin-3-ol

The title compound is prepared from (3R,4R,5S)-4-[4-(2-methoxyethoxymethyl)phenyl]-3-[4-(3-methoxypropyl)-2,2-dimethyl-2H-chromen-6-ylmethoxy]-1-(toluene-4-sulphonyl)-5-triisopropylsilanyloxypiperidine in analogy to method J and identified on the basis of the Rf.

### b) (3R,4R,5S)-4-[4-(2-Methoxyethoxymethyl)phenyl]-3-[4-(3-methoxypropyl)-2,2-dimethyl-2H-chromen-6-ylmethoxy]-1-(toluene-4-sulphonyl)-5-triisopropylsilanyloxypiperidine

The title compound is prepared from {([(3R,4R,5S)₃-[4-(3-methoxypropyl)-2,2-dimethyl-2H-chromen-6-ylmethoxy]-1-(toluene-4-sulphonyl)-5-triisopropylsilanyloxypiperidin-4-yl]-phenyl}methanol in analogy to Example 22c and identified on the basis of the Rf.

### c) {4-[(3R,4R,5S)-3-[4-(3-Methoxypropyl)-2,2-dimethyl-2H-chromen-6-ylmethoxy]-1-(toluene-4-sulphonyl)-5-triisopropylsilanyloxypiperidin-4-yl]phenyl}methanol

A solution of 8.060 g of methyl 4-[(3R,4R,5S)-3-[4-(3-methoxypropyl)-2,2-dimethyl-2H-chromen-6-ylmethoxy]-1-(toluene-4-sulphonyl)-5-triisopropylsilanyloxypiperidin-4-yl]-benzoate in 50 ml of diethyl ether is added dropwise to a suspension of 0.759 g of lithium aluminium hydride in 50 ml of diethyl ether at 0° C. After the addition is complete, the reaction mixture is stirred at 0° C. for 1 hour. Water, 4M sodium hydroxide solution and water are successively added to the reaction mixture, the resulting solid is filtered off through Hyflo, the filter cake is washed with diethyl ether, and the filtrate is evaporated. The title compound is obtained from the residue by flash chromatography (SiO₂ 60F) and identified on the basis of the Rf.

### d) Methyl 4-[(3R,4R,5S)-3-[4-(3-methoxypropyl)-2,2-dimethyl-2H-chromen-6-ylmethoxy]-1-(toluene-4-sulphonyl)-5-triisopropylsilanyloxypiperidin-4-yl]benzoate

The title compound is obtained from methyl 4-[(3R,4R,5S-3-hydroxy-1-(toluenesulphonyl)-5-triisopropylsilanyloxypiperidin-4-yl]benzoate and 6-bromomethyl-4-(3-methoxypropyl) 2,2-dimethyl-2H-chromene in analogy to method D and identified on the basis of the Rf.

### e) Methyl 4-[(3R,4R,5S)-3-hydroxy-1-(toluenesulphonyl)-5-triisopropylsilanyloxypiperidin-4-yl]benzoate

130 ml of saturated aqueous sodium bicarbonate solution are added to a solution of 6.170 g of methyl 4-(3R,4R,5S-3-hydroxy-5-triisopropylsilanyloxypiperidin-4-yl)benzoate in 1.30 ml of ethyl acetate. 3.210 g of p-toluenesulphonyl chloride are added in portions while stirring vigorously. The reaction mixture is stirred at 0° C. for a further 2 hours and then the phases are separated. The aqueous phase is back-extracted with ethyl acetate, and the combined organic phases are washed with brine, dried with sodium sulphate and evaporated. The title compound is obtained as a white foam from the residue by flash chromatography (SiO₂ 60F). Rf=0.63 (EtOAc/heptane 1:2); Rt=6.28 (gradient I).

### f) Methyl 4-((3R,4R,5S-3-hydroxy-5-triisopropylsilanyloxypiperidin-4-yl)benzoate

The title compound is obtained as a white foam from 5.400 g of benzyl (3R,4R,5S)-3-hydroxy-4-(4-methoxycarbonyl phenyl)-5-triisopropylsilanyloxypiperidine-1-carboxylate (Example 41) in analogy to method B. Rf=0.36 (dichloromethane/methanol/25% conc. ammonia 200:20:1); Rt=4.36 (gradient I).

### g) 6-Bromomethyl-4-(3-methoxypropyl)-2,2-dimethyl-2H-chromene

1.560 ml of trimethylsilyl bromide are added dropwise to a solution of 2.067 g of [4-(3-methoxypropyl)-2,2-dimethyl-2H-Chromen-6-yl]methanol in chloroform at room temperature. The reaction solution is stirred at room temperature for 30 minutes and then evaporated. The title compound is obtained from the residue by flash chromatography (SiO₂ 60F) and identified on the basis of the Rf.

### h) [4-(3-Methoxypropyl)-2,2-dimethyl-2H-chromen-6-yl]methanol

0.0278 g of lithium borohydride is added in portions to a solution of 0.316 g of 4-(3-methoxypropyl)-2,2-dimethyl-2H-chromene-6-carbaldehyde in 5 ml of dry tetrahydrofuran at 0° C. The reaction mixture is stirred at 0° C. for 2 hours and then 5 ml of methanol and 0.5 ml are added, and the mixture is evaporated. The title compound is obtained as a white solid and employed without further purification in the next stage. Rt=4.00 (gradient I).

### i) 4-(3-Methoxypropyl)-2,2-dimethyl-2H-chromene-6-carbaldehyde

A solution of 1.000 g of 6-bromo-4-(3-methoxypropyl)-2,2-dimethyl-2H-chromene in 12 ml of dry tetrahydrofuran is cooled to −78° C. 1.77 ml of n-butyllithium solution (1.6M in hexane) are added dropwise at −78°-−70° C., and the reaction solution is then stirred at −78° C. for 30 minutes. 0.398 ml of N,N-dimethylformamide is added dropwise, the solution is stirred at the same temperature for a further 45 minutes, and then saturated aqueous ammonium chloride solution is added. The mixture is warmed to room temperature and extracted with tert-butyl methyl ether. The combined organic extracts are washed with brine, dried with sodium sulphate and evaporated. The total compound is obtained as a colourless oil from the residue by flash chromatography (SiO₂ 60F). Rt=4.79 (gradient I).

### j) 6-Bromo-4-(3-methoxypropyl)2,2-dimethyl-2H-chromene

A solution of 17.10 g of 6-bromo-4-(3-methoxyprop-1-ynyl-2,2-dimethyl-2H-chromene in 600 ml of ethyl acetate is mixed with 3.10 ml of acetic acid. The reaction mixture is cooled to −15 to −10° C., and 8.88 g of 10% Pd/C are added and a hydrogen atmosphere is provided by means of a balloon. The reaction mixture is then stirred at 0-25° C. for 1 hour. The catalyst is subsequently filtered off through Hyflo, and the filtrate is washed with saturated aqueous sodium bicarbonate solution. The organic phase is dried with sodium sulphate and evaporated. The title compound is obtained as a yellowish oil from the residue by flash chromatography (SiO₂ 60F). Rf=0.28 (EtOAc/heptane 1:5); Rt=5.66 (gradient I).

### k) 6-Bromo-4-(3-methoxyprop-1-ynyl)-2,2-dimethyl-2H-chromene

497 ml of triethylamine are added to a suspension of 2.518 g of bis(triphenylphosphine)-palladium(II) chloride and 0.683 g of copper(1) iodide in 500 ml of dry tetrahydrofuran at room temperature. A solution of 29.60 g of 6-bromo-2,2-dimethyl-2H-chromen-4-yl trifluoromethanesulphonate and 7.698 g of methyl 2-propynyl ether in 200 ml of tetrahydrofuran is added, and the reaction mixture is heated to 50° C. The mixture is stirred at this temperature for 1.5 hours and then evaporated. The title compound is obtained as a yellowish oil from the residue by flash chromatography (SiO₂ 60F). Rf=0.45 (EtOAc/heptane 1:10); Rt=5.44 (gradient I).

### l) 6-Bromo-2,2-d-dimethyl-2H-chromen-4-yl trifluoromethanesulphonate

20.0 ml of N,N-diisopropylethylamine are added to a solution of 21.0.0 g of 6-bromo-2,2-dimethylchroman-4-one [99853-21-1] in 200 ml of dichloromethane at −15° C. 20.6 ml of trifluoromethanesulphonic anhydride are added dropwise over the course of 10 minutes at −15° C., and the reaction solution is then stirred at room temperature for 16 hours. Water is added to the reaction mixture, the phases are separated, and the aqueous phase is back-extracted with dichloromethane. The combined organic phases are dried with sodium sulphate and evaporated. The title compound is obtained as a yellowish oil from the residue by flash chromatography (SiO₂ 60F). Rf=0.55 (EtOAc/heptane 1:10); Rt=5.84 (gradient I).

### Example 120

### (R)-1-Methoxy-3-{(3S,4R,5R)-4-[4-(2-methoxyethoxymethyl)phenyl]-5-[4-(3-methoxypropyl)-2,2-dimethyl-2H-chromen-6ylmethoxy]piperidin-3-yloxy}propan-2-ol

The title compound is obtained starting from (3S,4S,5R)-4-[4-(2-methoxyethoxymethyl)-phenyl]-5-[4-(3-methoxypropyl)-2,2-dimethyl-2H-chromen-6-ylmethoxy]-1-(toluene-4-sulphonyl)piperidin-3-ol (Example 119a) in analogy to the process described in Example 22.

### Example 121

### (R)-4{(3S,4R,5R)-4-[4-(2-methoxyethoxymethyl)Phenyl]-5-[4-(3-methoxypropyl).3,4-dihydro-2H-benzo[1,4]oxazin-6-ylmethoxy]piperidin-3-yl}-butan-2-ol

The title compound is prepared from (R)-4-[(3S,4R,5R)-4-[4-(2-methoxyethoxymethyl)-phenyl]-5-[4-(3-methoxypropyl)-3,4-dihydro-2H-benzo[1,4]oxazin-6-ylmethoxy]-1-(toluene-4-sulphonyl)piperidin-3-yl]butan-2-ol in analogy to method L.

The starting materials are prepared as follows:

### a) (R)-4-[(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yl]butan-2-ol

The title compound is prepared from 6-[(3R,4R,5S)-4-[4-(2-methoxyethoxymethyl)phenyl]-1-(toluene-4-sulphonyl)-5-((R)-3-triisopropylsilanyloxybutyl)piperidin-3-yloxymethyl]-4-(3-methoxypropyl)-3,4-dihydro-2H-benzo[1,4]oxazine in analogy to method J and identified on the basis of the Rf.

### b) 6-[(3R,4R,5S)-4-[4-(2-Methoxyethoxymethyl)phenyl]-1-(toluene-4-sulphonyl)-5-((R)-3-triisopropylsilanyloxybutyl)piperidin-3-yloxymethyl]-4-(3-methoxypropyl)-3,4-dihydro-2H-benzo[1,4]oxazine

The title compound is prepared from [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ylmethyl]triphenylphosphonium bromide and (R)-2-triisopropylsilanyloxypropionaldehyde [178802-51-2] in analogy to the process described in Example 114g-h and identified on the basis of the Rf.

### c) [(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl]triphenylphosphonium bromide

1.66 mmol of triphenylphosphine are added to a stirred solution of 1.37 mmol of 6-[(3R,4R,5S)-5-bromomethyl-4-[4-(2-methoxyethoxymethyl)phenyl]-1-(toluene-4-sulphonyl)piperidin-3-yloxymethyl]-4-(3-methoxypropyl)-3,4-dihydro-2H-benzo[1,4]oxazine in 2 ml of acetonitrile, and the mixture is kept at 80° C. for 18 hours. The reaction mixture is cooled to room temperature, and the solid is filtered off with suction. The title compound is identified on the basis of the Rf.

### d) 6-[(3R,4R,5S)-5-Bromomethyl-4-[4-(2-methoxyethoxymethyl)phenyl]-1-(toluene-4-sulphonyl)piperidin-3-yloxymethyl]-4-(3-methoxypropyl)-3,4-dihydro-2H-benzo[1,4]oxazine

20 mmol of lithium bromide are added to a solution of 2 mmol of (3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl methanesulphonate in 5 ml of N,N-dimethylformamide, and the mixture is heated at 65° C. for 14 hours. The reaction mixture is cooled to room temperature and quenched with water. It is extracted with tert-butyl methyl ether (3×), and the combined organic phases are dried with sodium sulphate and evaporated. The title compound is identified on the basis of the Rf from the residue by flash chromatography (SiO₂ 60F).

### e) (3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ylmethyl methanesulphonate

6 mmol of methanesulphonyl chloride are added to a solution of 3 mmol of [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yl]methanol (Example 68c) and 15 mmol of triethylamine in 30 ml of dichloromethane at 0° C., and the mixture is stirred at 0° C. for 1 hour. It is diluted with dichloromethane and washed with 1N HBr. The organic phase is dried with sodium sulphate and evaporated. The title compound is used without further purification in the next stage.

The following compound is prepared in an analogous manner to the process described in Example 121:

### Example

### 122 (R-4-(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yl)butan-2-ol

Using (S)-2-triisopropylsilanyloxypropionaldehyde [135614-51-7]

### Example 123

### N—((R)-2-{(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]piperidin-3-yloxy}-methylethyl)acetamide

The title compound is prepared from (S)-2-[(3S,4R,5R)-4-[4-(2-methoxyethoxymethyl)-phenyl]-5-[4-(3-methoxypropyl)-3,4-dihydro-2H-benzo[1,4]oxazin-6-ylmethoxy]-1-(toluene-4-sulphonyl)piperidin-3-yloxy]-1-methylethyl toluene-4-sulphonate in analogy to the process described in Example 72.

The starting material is prepared as follows:

### a) (S)-2-[(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]-1-methylethyl toluene-4-sulphonate

The title compound is identified on the basis of the Rf from (S)-1-[(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-toluene-4-sulphonyl)piperidin-3-yloxy]propan-2-ol in analogy to method H.

### b) (S)-1-[(3S,4R,5R)-4-[4-(2-Methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]propan-2-ol

The title compound is prepared from (3S,4S,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-ol (Example 22c) using (S)-1-oxiranymethyl toluene-4-sulphonate [70987-78-9] in analogy to the process described in Example 31, and identified on the basis of the Rf.

### Example 124

### 6-{(3R,4R,5S)-5-((R-2-Ethoxypropoxy)-4-[4-(2-methoxyethoxymethyl)phenyl]piperidin-3-yloxymethyl}-4-(3-methoxypropyl)-3,4-dihydro-2H-benzo[1,4]oxazine

The title compound is prepared from 6-[(3R,4R,5S)-5-((R)-2-ethoxypropoxy)-4-[4-(2-methoxyethoxymethyl)phenyl]-1-(toluene-4-sulphonyl)piperidin-3-yloxymethyl]-4-(3-methoxypropyl)-3,4-dihydro-2H-benzo[1,4]oxazine in analogy to method L.

The starting materials are prepared as follows:

### a) 6-[(3R,4R,5S)-5-((R)-2-Ethoxypropoxy)-4-[4-(2-methoxyethoxymethyl)phenyl]-1-(toluene-4-sulphonyl)piperidin-3-yloxymethyl]-4-(3-methoxypropyl)-3,4-dihydro-2H-benzo[1,4]oxazine

1.0 mmol of (R)-1-[(3S,4R,5R)-4-[4-2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]-propan-2-ol and 1.5 mmol of ethyl iodide are reacted in analogy to method D. The title compound is identified on the basis of the Rf.

### b) (R)-1-[(3S,4R,5R)-4-[4-(2-Methoxyethoxymethyl)phenyl]-5-[4-(3-methoxypropyl) 3,4-dihydro-2H-benzo[1,4]oxazin-6-ylmethoxy]-1-(toluene-4-sulphonyl)piperidin-3-yloxy]propan-2-ol

The title compound is prepared from (3S,4S,5R)[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)-piperidin-3-ol (Example 22c) in analogy to the process described in Example 31, and identified on the basis of the Rf.

The following compound is prepared in an analogous manner to the process described in Example 124:

### Example

### 127 6-[(3R,4R,5S)-4-[4-(2-Methoxy-ethoxymethyl)phenyl]-5-((R)-2-methoxy-propoxy)-piperidin-3-yloxymethyl]-4-(3-methoxy-propyl)-3,4-dihydro-2H-benzo[1,4]oxazine

Using methyl iodide

### Example 128

### 1-{(3S,4R,5R)-4-[4-(2-Methoxy-ethoxymethyl)-phenyl]-5-[4-(3-methoxy-propyl)-3,4-dihydro-2H-benzo[1,4]oxazin-6-ylmethoxy]-piperidin-3-yloxy}-2-methyl-propan-2-ol

The title compound is prepared in analogy to method L from 0.51 g of 1-[(3S,4R,5R)[4-(2-methoxy-ethoxymethyl)-phenyl]-5-[4-(3-methoxy-propyl)-3,4-dihydro-2H-benzo[1,4]oxazin-6-ylmethoxy]-1-(toluene-4-sulphonyl)-piperidin-3-yloxy]-2-methyl-propan-2-ol.

The starting material is prepared as follows:

### a) 1-[(3S,4R,5R)-4-[4-(2-Methoxy-ethoxymethyl)-phenyl]-5-[4-(3-methoxy-propyl)-3,4-dihydro-2H-benzo[1,4]oxazin-6-ylmethoxy]-1-(toluene-4-sulphonyl)-piperidin-3-yloxy]-2-methyl-propan-2-ol

1.67 ml of methyl magnesium bromide (3M in diethyl ether) is added dropwise to a solution of 0.73 g of methyl [(3S,4R,5R)-4-[4-(2-methoxyethoxymethyl)phenyl]-5-[4-(3-methoxypropyl)-3,4-dihydro-2H-benzo[1,4]oxazin-6-ylmethoxy]-1-(toluene-4-sulphonyl)piperidin-3-yloxy]acetat (Example 32c) in 6.7 ml of tetrahydrofuran at 0° C. and then the mixture is stirred at 50° C. for 1 hour. The reaction mixture is cooled to 0° C. and quenched with 1M aqueous potassium bisulphate solution. The mixture is partitioned between ethyl acetate and water—the aqueous layer is re-extracted with ethyl acetate. The combined organic phases are washed with brine, dried with sodium sulphate and evaporated. The title compound is obtained as a yellowish oil from the residue by flash chromatography (SiO₂ 60F). Rf=0.11 (EtOAc/heptane 1:1); Rt=5.05 (gradient I).

### Example 129

### (R)-1-{(3S,4R,5R)₄₋₄-(2-Methoxy-ethylsulphanylmethyl)-phenyl]-5-[4-(3-methoxy-propyl)-3,4-dihydro-2H-benzo[1,4]oxazin-6-ylmethoxy]-piperidin-3-yloxy}-propan-2-ol

0.097 g of lithium aluminium hydride are added to a solution of 0.40 g of (R)-1-[(3S,4R,5R)-4-[4-(2-methoxy-ethylsulphanylmethyl)-phenyl]-5-[4-(3-methoxy-propyl)-3,4-dihydro-2H-benzo[1,4]oxazin-6-ylmethoxy]-1-(toluene-4-sulphonyl)-piperidin-3-yloxy]-propan-2-ol in 6 ml of tetrahydrofuran at room temperature and then the mixture is stirred at 50° C. for 40 hours. (Note: an additional 0.097 g of lithium aluminium hydride were added after 24 hours.) The mixture was cooled to room temperature and diluted with tert-butyl methyl ether. Water, 4M sodium hydroxide solution and again water are successively added to the reaction mixture—the resulting solid is filtered off through Hyflo, the filter cake is washed with tert-butyl methyl ether, and the filtrate is evaporated. The title compound is obtained from the residue by flash chromatography (SiO₂ 60F).

The starting materials are prepared as follows:

### a) (R)-1-[(3S,4R,5R)-4-[4-(2-methoxy-ethylsulphanylmethyl)phenyl]-5-[4-(3-methoxypropyl)-3,4-dihydro-2H-benzo[1,4]oxazin-6-ylmethoxy]-1 4toluene-4-sulphonyl)-piperidin-3-yloxy]-propan-2-ol

0.81 g of 6-[(3R,4R,5S)-4-[4-(2-methoxy-ethylsulphanylmethyl)phenyl]-5-((R)-1-oxiranylmethoxy)-1-(toluene-4-sulphonyl)piperidin-3-yloxymethyl]-4-(3-methoxy-propyl)-3,4-dihydro-2H-benzo[1,4]oxazine and 0.11 g of sodium borohydride are reacted in analogy to Example 43a. The title compound is obtained as a yellow oil. Rf=0.20 (EtOAc/heptane 3:1); Rt=5.09 (gradient I).

### b) 6-[(3R,4R,5S)-4-[4-(2-Methoxy-ethylsulphanylmethyl)phenyl]-5-((R)-1-oxiranylmethoxy)-1-(toluene-4-sulphonyl)-piperidin-3-yloxymethyl]-4-(3-methoxy-propyl)-3,4-dihydro-2H-benzo[1,4]oxazine

0.80 g of (3S,4S,5R)-4-[4-(2-methoxy-ethylsulphanylmethyl)-phenyl]-5-[4-(3-methoxy-propyl) 3,4-dihydro-2H-benzo[1,4]oxazin-6-ylmethoxy]-1-(toluene-4-sulphonyl)-piperidin-3-ol and 0.55 g of (R)-1-oxiranymethyl toluene-4-sulphonate [113826-06-5] are reacted in analogy to Example 31a. The title compound is obtained as an orange-brown oil. Rf=0.20 (EtOAc/heptane 3:1); Rt=5.29 (gradient I).

### c) (3S,4S,5R)-4-[4-(2-Methoxy-ethylsulphanylmethyl)-phenyl]-5-[4-(3-methoxy-propyl)-3,4-dihydro-2H-benzo[1,4]oxazin-6-ylmethoxy]-1-(toluene-4-sulphonyl)-piperidin-3-ol

2.04 g of 6-[(3R,4R,5S)-4-[4-(2-methoxy-ethylsulphanylmethyl)-phenyl]-1-(toluene-4-sulphonyl)-5-triisopropylsilanyloxy-piperidin-3-yloxymethyl]-4-(3-methoxy-propyl)-3,4-dihydro-2H-benzo[1,4]oxazine are reacted in analogy to method J. The title compound is obtained as a yellow oil. Rf=0.20 (EtOAc/heptane 1:1); Rt=4.92 (gradient I).

### d) 6-[(3R,4R,5S)-4-[4-(2-Methoxy-ethylsulphanylmethyl)phenyl]-1-(toluene-4-sulphonyl)-5-triisopropylsilanyloxy-piperidin-3-yloxymethyl]-4-(3-methoxy-propyl)-3,4-dihydro-2H-benzo[1,4]oxazine

0.16 g of sodium hydride (60% dispersion in oil) are added to a solution of 2.27 g of 2-4-[(8R,4R,5S)₃-[4-(3-methoxy-propyl)-3,4-dihydro-2H-benzo[1,4]oxazin-6-ylmethoxy]-1-(toluene-4-sulphonyl)-5-triisopropylsilanyloxy-piperidin-4-yl]-benzylsulphanyl)ethanol and 0.34 ml of methyl iodide in 25 ml of tetrahydrofuran at 0° C. The reaction mixture is stirred at 0° C. for 1 hour and then at room temperature for 16 hours. The mixture quenched by pouring into a mixture of 1:1 ice-water/brine and extracting three times with dichloromethane—the combined organic layers are washed with brine, dried with sodium sulphate and evaporated. The title compound is obtained as a yellow oil from the residue by flash chromatography (SiO₂ 60F). Rf=0.50 (EtOAc/heptane 1:1); Rt=32.09 (gradient II).

### e) 2-{4-[(3R,4R,5S)-3-[4-(3-Methoxy-propyl)-3,4-dihydro-2H-benzo[1,4]oxazin-6-ylmethoxy]-1 4toluene-4-sulphonyl)-5-triisopropylsilanyloxy-piperidin-4-yl]-benzylsulphanyl}-ethanol

A mixture of 2.2 g of 6-[(3R,4R,5S)-4-(4-chloromethylphenyl)-1-(toluene sulphonyl)-5-triisopropylsilanyloxypiperidin-3-yloxymethyl]-4-(3-methoxypropyl)-3,4-dihydro-2H-benzo[1,4]oxazine (Example 4c), 0.23 ml of 2-mercaptoethanol and 0.60 g of potassium carbonate in 10 ml of N,N-dimethylformamide is stirred at room temperature for 18 hours. The reaction mixture is diluted with water and extracted three times with tert-butyl methyl ether—the combined organic layers are washed with brine, dried with sodium sulphate and evaporated. The crude title compound is obtained as a yellow oil. Rt=22.92 (gradient II).

1. A compound of the general formula (II) 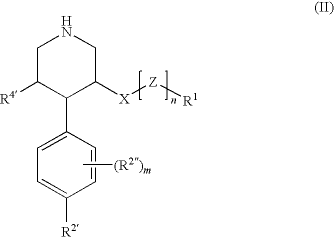
in which
(B) R¹ is aryl when X is —O—CHR⁵—CO—NR⁶—; or
(C) R¹ is aryl when Z is -Alk-NR⁶—, where Alk is C₁₋₈alkylene, and n is 1; or
(D) R¹ is aryl which is substituted by 1-4 acetamidinyl-C₁₋₈alkyl, acyl-C₁₋₈alkoxy-C₁₋₈alkyl, (N-acyl)-C₁₋₈alkoxy-C₁₋₈alkylamino, C₁₋₈alkoxy, C₁₋₈alkoxy-C₁₋₈alkoxy, C₁₋₈alkoxy-C₁₋₈alkoxy-C₁₋₈alkyl, C₁₋₈alkoxy-C₁₋₈alkyl, (N—C₁₋₈alkoxy)-C₁₋₈alkylaminocarbonyl-C₁₋₈alkoxy, (N—C₁₋₈alkoxy)-C₁₋₈alkylaminocarbonyl-C₁₋₈alkyl, C₁₋₈alkoxy-C₁₋₈alkylcarbamoyl, C₁₋₈alkoxy-C₁₋₈alkylcarbonyl, C₁₋₈alkoxy-C₁₋₈alkylcarbonylamino, 1-C₁₋₈alkoxy-C₁₋₈alkylimidazol-2-yl, 2-C₁₋₈alkoxy-C₁₋₈alkyl-4-oxoimidazol-1-yl, 1-C₁₋₈alkoxy-C₁₋₈alkyltetrazol-5-yl, 5-C₁₋₈alkoxy-C₁₋₈alkyltetrazol-1-yl, 6-alkoxyaminocarbonyl-C₁₋₈alkoxy, C₁₋₈alkoxyaminocarbonyl-C₁₋₈alkyl, C₁₋₈alkoxycarbonyl, C₁₋₈alkoxycarbonyl-C₁₋₈alkoxy, C₁₋₈alkoxycarbonyl-C₁₋₈alkyl, C₁₋₈alkoxycarbonylamino-C₁₋₈alkoxy, C₁₋₈alkoxycarbonylamino-C₁₋₈alkyl, C₁₋₈alkyl, (N—C₁₋₈alkyl)-C₁₋₈alkoxy-C₁₋₈alkylcarbamoyl, (N—C₁₋₈alkyl)-C₁₋₈alkoxy-C₁₋₈alkylcarbonylamino, (N—C₁₋₈alkyl)-C₁₋₈alkoxycarbonylamino, (N—C₁₋₈alkyl)-C₀₋₈alkylcarbonylamino-C₁₋₈alkoxy, (N—C₁₋₈alkyl)-C₀₋₈alkylcarbonylamino-C₁₋₈alkyl, (N—C₁₋₈alkyl)-C₁₋₈alkylsulphonylamino-C₁₋₈alkoxy, (N—C₁₋₈alkyl)-C₁₋₈alkylsulphonylamino-C₁₋₈alkyl, C₁₋₈alkylamidinyl, C₁₋₈alkylaminocarbonyl-C₁₋₈alkoxy, di-C₁₋₈alkylaminocarbonyl-C₁₋₈alkoxy, C₁₋₈alkylaminocarbonyl-C₁₋₈alkoxy-C₁₋₈alkyl, C₁₋₈alkylaminocarbonyl-C₁₋₈alkyl, C₁₋₈alkylaminocarbonylamino-C₁₋₈alkoxy, C₁₋₈alkylaminocarbonylamino-C₁₋₈alkyl, di-C₁₋₈alkylaminocarbonyl-C₁₋₈alkyl, C₁₋₈alkylamino-C₂₋₈alkoxy, di-C₁₋₈alkylamino-C₂₋₈alkoxy, C₁₋₈alkylamino-C₁₋₈alkyl, di-C₁₋₈alkylamino-C₁₋₈alkyl, C₁₋₈alkylcarbamoyl, di-C₁₋₈alkylcarbamoyl, C₀₋₈alkylcarbonylamino-C₁₋₈alkoxy, C₀₋₈alkylcarbonylamino, C₀₋₈alkylcarbonylamino-C₁₋₈alkyl, C₁₋₈alkylcarbonyloxy-C₁₋₈alkoxy, C₁₋₈alkyl-carbonyloxy-C₁₋₈alkyl, C₁₋₈alkylsulphonyl, C₁₋₈alkylsulphonyl-C₁₋₈alkoxy, C₁₋₈alkylsulphonyl-C₁₋₈alkyl, C₁₋₈alkylsulphonylamino-C₁₋₈alkoxy, C₁₋₈alkylsulphonylamino-C₁₋₈alkyl, carbamoyl, carbamoyl-C₁₋₈alkoxy, carbamoyl-C₁₋₈alkyl, carboxy-C₁₋₈alkoxy, carboxy-C₁₋₈alkoxy-C₁₋₈alkyl, carboxy-C₁₋₈alkyl, cyano, cyano-C₁₋₈alkoxy, cyano-C₁₋₈alkyl, C₃₋₈cycloalkyl-C₁₋₈alkoxy, C₃₋₈cycloalkyl-C₁₋₈alkyl, C₃₋₈cycloalkylcarbonylamino-C₁₋₈alkoxy, C₃₋₈cycloalkylcarbonylamino-C₁₋₈alkyl, O,N-dimethylhydroxylamino-C₁₋₈alkyl, halogen, hydroxy-C₁₋₈alkoxy-C₁₋₈alkoxy, hydroxy-C₁₋₈alkoxy-C₁₋₈alkyl, hydroxy-C₁₋₈alkyl, (N-hydroxy)-C₁₋₈alkylaminocarbonyl-C₁₋₈alkoxy, (N-hydroxy)-C₁₋₈alkylaminocarbonyl-C₁₋₈alkyl, (N-hydroxy)aminocarbonyl-C₁₋₈alkoxy, (N-hydroxy)-aminocarbonyl-C₁₋₈alkyl, 2-oxoxazolidinyl-C₁₋₈alkoxy, 2-oxoxazolidinyl-C₁₋₈alkyl, O-methyloximyl-C₁₋₈alkyl or trifluoromethyl; or
(E) R¹ is aryl which is substituted by 1-4 3-acetamidomethylpyrrolidinyl, 3-C₁₋₈alkoxy-C₁₋₈alkylpyrrolidinyl, 3,4-dihydroxypyrrolidinyl, 2,6-dimethylmorpholinyl, 3,5-dimethylmorpholinyl, dioxanyl, dioxolanyl, 4,4-dioxothiomorpholinyl, dithianyl, dithiolanyl, 2-hydroxymethylpyrrolidinyl, 4-hydroxypiperidinyl, 3-hydroxypyrrolidinyl, imidazolylalkoxy, imidazolylalkyl, 2-methylimidazolylalkoxy, 2-methylimidazolylalkyl, 3-methyl-[1,2,4]-oxadiazol-5-ylalkoxy, 5-methyl-[1,2,4]-oxadiazol-3-ylalkoxy, 3-methyl-[1,2,4]-oxadiazol-5-ylalkyl, 5-methyl-[1,2,4]-oxadiazol-3-ylalkyl, 4-methylpiperazinyl, 5-methyl-tetrazol-1-ylalkoxy, 5-methyltetrazol-1-ylalkyl, morpholinyl, [1,2,4]-oxadiazol-5-ylalkoxy, [1,2,4]-oxadiazol-5-ylalkyl, oxazol-4-ylalkoxy, oxazol-4-ylalkyl, 2-oxo-[1,3]-oxazinyl, 2-oxoxazolidinyl, 2-oxoimidazolidinyl, 2-oxopyrrolidinyl, 4-oxopiperidinyl, 2-oxopyrrolidinylalkoxy, 2-oxopyrrolidinylalkyl, 2-oxotetrahydropyrimidinyl 4-oxothiomorpholinyl, piperazinyl, piperidinyl, pyrrolidinyl, pyrrolyl, [1,2,4]-triazol-1-ylalkoxy, [1,2,4]-triazol-4-ylalkoxy, [1,2,4]-triazol-1-ylalkyl, [1,2,4]-triazol-4-ylalkyl, tetrazol-1-ylalkoxy, tetrazol-2-ylalkoxy, tetrazol-5-ylalkoxy, tetrazol-1-ylalkyl, tetrazol-2-ylalkyl, tetrazol-5-ylalkyl, thiazol-4-ylalkoxy, thiazol-4-ylalkyl or thiomorpholinyl; or
(F) R¹ is heterocyclyl, optionally substituted by oxo or oxide or as indicated under (D) or (E), especially azepanyl, benzo[1,3]dioxolyl, benzofuranyl, benzoimidazolyl, 4H-benzo[1,4]oxazinyl, benzoxazolyl, 4H-benzo[1,4]thiazinyl, 1H-quinolinyl, 2H-chromenyl, dihydrobenzo[e][1,4]diazepinyl, dihydrobenzofuranyl, 3,4-dihydro-2H-benzo[1,4]oxazinyl, dihydro-3H-benzo[1,4]oxazinyl, dihydrobenzo[d][1,3]oxazinyl, dihydro-2H-benzo[1,4]thiazinyl, dihydro-2H-1λ6-benzo[1,4]thiazinyl, dihydro-1H-quinazolinyl, 1a,7b-dihydro-1H-cyclopropa[c]chromenyl, dihydroimidazolyl, 1,3-dihydroindolyl, 2,3-dihydroindolyl, dihydro-1H-pyrido[2,3-b][1,4]oxazinyl, indazolyl, indolyl, 3H-isobenzofuranyl, [1,5]naphthyridyl, oxazolyl, phthalazinyl, piperidinyl, pyrazolyl, 1H-pyrido[2,3-b][1,4]oxazinyl, pyridyl, 1H-pyrrolizinyl, 1H-pyrrolo[2,3-b]pyridyl, pyrrolyl, tetrahydrobenzo[e][1,4]diazepinyl, 3H-thieno[2,3-d]pyrimidinyl, tetrahydroquinoxalinyl, 1,1a,2,7b-tetrahydrocyclopropa[c]chromenyl, tetrahydropyranyl or triazinyl;
R²′ is C₂₋₈alkenyloxy-C₁₋₈alkyl, C₁₋₈alkoxy-C₁₋₈alkyl, C₁₋₈alkoxy-C₁₋₈alkylamino-C₁₋₈alkyl, C₁₋₈alkoxy-C₁₋₈alkylsulphanyl-C₁₋₈alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₁₋₈alkylsulphonyl-C₁₋₈alkoxy-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, optionally halogen-substituted C₁₋₈alkoxy-C₁₋₈alkoxy-C₁₋₈alkyl, or (oxygen-heterocyclyl)-C₀₋₈alkoxy-C₁₋₈alkyl;
R²″ is halogen,
R⁴′ is a) optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈-alkylated amino-C₁₋₈alkoxy, optionally N—C₁₋₈-alkylated C₁₋₈alkoxy-C₁₋₈alkylamino-C₁₋₈alkoxy, optionally N-mono- or N,N-di-C₁₋₈-alkylated amino-C₀₋₈alkylcarbonyl-C₁₋₈alkoxy, hydroxy-C₀₋₈alkylcarbonyl-C₀₋₈alkoxy, C₁₋₈alkoxy-C₀₋₈alkylcarbonyl-C₀₋₈alkoxy, C₁₋₈alkylcarbonylamino-C₁₋₈alkoxy, cyano-C₁₋₈alkoxy, C₃₋₈cycloalkyl-C₀₋₈alkoxy, heterocyclyl-C₀₋₈alkoxy, optionally N—C₁₋₈-alkylated heterocyclyl-C₀₋₈alkylamino-C₀₋₈alkylcarbonyl-C₀₋₈alkoxy, C₁₋₈alkylsulphonyl-C₁₋₈alkoxy, C₂₋₈alkinyloxy, heterocyclyl-C₂₋₈alkinyloxy, optionally N-mono- or N,N-di-C₁₋₈alkylated amino-C₂₋₈alkinyloxy, N-mono- or N,N-di-C₁₋₈-alkylated aminocarbonyl-C₂₋₈alkinyloxy, heterocyclylcarbonyl-C₀₋₈alkoxy, optionally N-mono- or N,N-di-C₁₋₈-alkylated amino-C₁₋₈alkyl, optionally N—C₁₋₈-alkylated C₁₋₈alkoxy-C₁₋₈alkylamino-C₁₋₈alkyl, optionally N-mono- or N,N-di-C₁₋₈-alkylated and optionally hydroxy-substituted amino-C₀₋₈alkylcarbonyl-C₀₋₈alkyl, optionally N—C₁₋₈-alkylated heterocyclyl-C₀₋₈alkylamino-C₀₋₈alkylcarbonyl-C₀₋₈alkyl, optionally halogen- or hydroxy-substituted C₁₋₈alkoxy-C₁₋₈alkyl, optionally halogen- and/or hydroxy-substituted C₁₋₈alkyl, optionally N—C₁₋₈-alkylated hydroxy-C₁₋₈alkylamino-C₁₋₈alkyl, heterocyclylcarbonyl-C₀₋₈alkyl, heterocyclylcarbonyl-C₀₋₈alkylamino-C₁₋₈alkyl, heterocyclyl-C₁₋₈alkyl, C₁₋₈alkoxycarbonylamino-C₁₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl; or additionally
b) is hydroxy if R²′ is not C₁₋₈alkyl;
R⁵ is acyl, C₂₋₈alkenyl, C₁₋₈alkyl, aryl-C₁₋₈alkyl or hydrogen;
R⁶ is acyl, C₁₋₈alkoxy-C₁₋₈alkyl, C₁₋₈alkyl or aryl-C₁₋₈alkyl or hydrogen;
R⁷ is C₁₋₈alkoxycarbonyl-C₁₋₈alkyl, C₁₋₈alkyl, carboxy-C₁₋₈alkyl or hydrogen;
X is a bond, oxygen or sulphur, where the bond originating from an oxygen or sulphur atom leads to a saturated C atom of the group Z, or is a group >CH—R⁵, >CHOR⁶, —O—CO—, >CO, >C═NOR⁷, —O—CHR⁵— or —O—CHR⁵—CO—NR⁶—;
Z is C₁₋₈alkylene, C₂₋₈alkenylene, hydroxy-C₁₋₈alkylidene, —O—, —S—, —O-Alk-, —S-Alk-, -Alk-O—,
-Alk-S— or -Alk-NR⁶—,
where Alk is C₁₋₈alkylene; and where
(a) if Z is —O-Alk- or —S-Alk-, then X is —CHR⁵—; and
(b) if X is a bond, then Z is C₂₋₈alkenylene, -Alk-O— or -Alk-S—;
m is O, or 2; and
n is 1 or, if X is —O—CO— or —O—CHR⁵—CO—NR⁶—, is 0 or 1
and its salt or compound in which one or more atoms are replaced by their stable, non-radioactive isotopes, especially pharmaceutically acceptable salt.

2. A compound according to claim 1, which corresponds to the general formula (IIA) 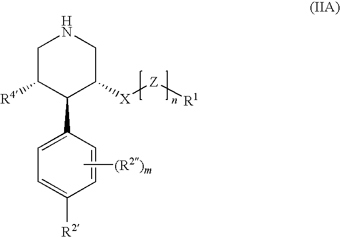
in which R¹, R²′, R²″, R⁴′, X and Z, and m and n have the meanings stated for the compound of the formula (II).

3. A compound according to claim 1, wherein
R⁴′ is a) optionally halogen and/or hydroxy-substituted C₁₋₈alkoxy, optionally halogen- and/or hydroxy-substituted C₁₋₈alkoxy-C₁₋₈alkoxy, optionally halogen-substituted hydroxy-C₁₋₈alkoxy, optionally N-mono- or N,N-di-C₁₋₈-alkylated amino-C₁₋₈alkoxy, optionally N-mono- or N,N-di-C₁₋₈-alkylated amino-C₀₋₈alkylcarbonyl-C₁₋₈alkoxy, C₁₋₈alkoxy-C₀₋₈alkylcarbonyl-C₀₋₈alkoxy, cyano-C₁₋₈alkoxy, C₁₋₈cycloalkyl-C₀₋₈alkoxy, heterocyclyl-C₀₋₈alkoxy, C₁₋₈alkylsulphonyl-C₁₋₈alkoxy, C₂₋₈alkinyloxy, heterocyclyl-C₂₋₈alkinyloxy, optionally N-mono- or N,N-di-C₁₋₈alkylated amino-C₂₋₈alkinyloxy, heterocyclylcarbonyl-C₀₋₈alkoxy, optionally N-mono- or N,N-di-C₁₋₈-alkylated amino-C₁₋₈alkyl, optionally N-mono- or N,N-di-C₁₋₈-alkylated and optionally hydroxy-substituted amino-C₀₋₈alkylcarbonyl-C₀₋₈alkyl, optionally halogen- or hydroxy-substituted C₁₋₁₈alkoxy-C₁₋₈alkyl, optionally halogen- and/or hydroxy-substituted C₁₋₈alkyl, heterocyclyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl; or additionally
b) is hydroxy if R²′ is not C₁₋₈alkyl.

4. Compound according to claim 1, wherein
R¹ is optionally substituted benzoimidazolyl or a substituted radical selected from 2H-chromenyl, 3,4-dihydro-2H-benzo[1,4]oxazinyl, 1a,7b-dihydro-1H-cyclopropa[c]chromenyl, indazolyl, indolyl, phenyl and 1,1a,2,7b-tetrahydrocyclopropa[c]chromenyl;
R²′ is C₂₋₈-alkenyloxy-C₁₋₈alkyl, C₁₋₈alkoxy-C₁₋₈alkyl, C₁₋₈alkoxy-C₁₋₈alkylsulphanyl-C₁₋₈-8alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, or optionally halogen-substituted C₁₋₈alkoxy-C₁₋₈alkoxy-C₁₋₈alkyl;
R⁴′ is hydroxy, optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈-alkylated amino-C₁₋₈alkoxy, heterocyclyl-C₀₋₈alkoxy, optionally N-mono- or N,N-di-C₁₋₈-alkylated amino-C₀₋₈alkylcarbonyl-C₁₋₈alkoxy, heterocyclylcarbonyl-C₀₋₈alkoxy, optionally N-mono- or N,N-di-C₁₋₈-alkylated and optionally hydroxy-substituted amino-C₀₋₈alkylcarbonyl-C₀₋₈alkyl, heterocyclyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl;
X is —O— or >CH—R⁵;
Z is C₁₋₈alkylene;
m is 0;
and
n is 1.

5-6. (canceled)

7. A method for preventing, for delaying the progression or for treating high blood pressure, heart failure, glaucoma, myocardial infarction, renal failure, restenoses or stroke, where a therapeutically effective amount of a compound of the general formula (II) according to claim 1 is used.

8. A pharmaceutical product comprising a compound of the general formula (II) according to claim 1, and usual excipients.

9. A pharmaceutical combination in the form of a product or of a kit composed of individual components consisting a) of a compound of the general formula (II) according to claim 1, and b) at least one pharmaceutical form whose active ingredient has a cardiovascular effect.

10. A compound according to claim 2, wherein
R⁴′ is a) optionally halogen and/or hydroxy-substituted C₁₋₈alkoxy, optionally halogen- and/or hydroxy-substituted C₁₋₈alkoxy-C₁₋₈alkoxy, optionally halogen-substituted hydroxy-C₁₋₈alkoxy, optionally N-mono- or N,N-di-C₁₋₈-alkylated amino-C₁₋₈alkoxy, optionally N-mono- or N,N-di-C₁₋₈-alkylated amino-C₀₋₈alkylcarbonyl-C₁₋₈alkoxy, C₁₋₈alkoxy-C₀₋₈alkylcarbonyl-C₀₋₈alkoxy, cyano-C₁₋₈ alkoxy, C₁₋₈cycloalkyl-C₀₋₈alkoxy, heterocyclyl-C₀₋₈alkoxy, C₁₋₈alkylsulphonyl-C₁₋₈alkoxy, C₂₋₈alkinyloxy, heterocyclyl-C₂₋₈alkinyloxy, optionally N-mono- or N,N-di-C₁₋₈-alkylated amino-C₂₋₈alkinyloxy, heterocyclylcarbonyl-C₀₋₈alkoxy, optionally N-mono- or N,N-di-C₁₋₈-alkylated amino-C₁₋₈alkyl, optionally N-mono- or N,N-di-C₁₋₈-alkylated and optionally hydroxy-substituted amino-C₀₋₈alkylcarbonyl-C₀₋₈alkyl, optionally halogen- or hydroxy-substituted C₁₋₈alkoxy-C₁₋₈alkyl, optionally halogen- and/or hydroxy-substituted C₁₋₈alkyl, heterocyclyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl; or additionally
b) is hydroxy if R²′ is not C₁₋₈alkyl.

11. Compound according to claim 2, wherein
R¹ is optionally substituted benzoimidazolyl or a substituted radical selected from 2H-chromenyl, 3,4-dihydro-2H-benzo[1,4]oxazinyl, 1a,7b-dihydro-1H-cyclopropa[c]chromenyl, indazolyl, indolyl, phenyl and 1,1a,2,7b-tetrahydrocyclopropa[c]chromenyl;
R²′ is C₂₋₈alkenyloxy-C₁₋₈alkyl, C₁₋₈alkoxy-C₁₋₈alkyl, C₁₋₈alkoxy-C₁₋₈alkylsulphanyl-C₁₋₈alkyl, C₁₋₈alkoxy-C₀₋₈alkyl-C₃₋₈cycloalkyl-C₀₋₈alkoxy-C₁₋₈alkyl, C₁₋₈alkyl, C₁₋₈alkylsulphanyl-C₁₋₈alkoxy-C₁₋₈alkyl, C₁₋₈alkylsulphanyl-C₁₋₈alkyl, C₃₋₈cycloalkyl-C₀₋₈alkoxy-C₁₋₈alkoxy-C₁₋₈alkyl, C₃₋₈cycloalkyl-C₀₋₈alkoxy-C₁₋₈alkyl, or optionally halogen-substituted C₁₋₈alkoxy-C₁₋₈alkoxy-C₁₋₈alkyl;
R⁴′ is hydroxy, optionally halogen- and/or hydroxy-substituted C₁₋₈alkoxy, optionally halogen- and/or hydroxy-substituted C₁₋₈alkoxy-C₁₋₈alkoxy, optionally N-mono- or N,N-di-C₁₋₈alkylated amino-C₁₋₁₈alkoxy, heterocyclyl-C₀₋₈alkoxy, optionally N-mono- or N,N-di-C₁₋₈-alkylated amino-C₀₋₈alkylcarbonyl-C₁₋₈alkoxy, heterocyclylcarbonyl-C₀₋₈alkoxy, optionally N-mono- or N,N-di-C₁₋₈-alkylated and optionally hydroxy-substituted amino-C₀₋₈alkylcarbonyl-C₀₋₈alkyl, heterocyclyl-C₀₋₈alkylcarbonyl-C₀₋₈alkyl, optionally halogen-substituted heterocyclyl-C₀₋₈alkylcarbonylamino-C₁₋₈alkyl, optionally halogen-substituted C₃₋₈cycloalkyl-C₀₋₈alkylcarbonylamino-C₁₋₈alkyl or optionally halogen-substituted C₁₋₈alkylcarbonylamino-C₁₋₈alkyl;
X is —O— or >CH—R⁵;
Z is C₁₋₈alkylene;
m is 0;
and
n is 1.

12. A method for preventing, for delaying the progression or for treating high blood pressure, heart failure, glaucoma, myocardial infarction, renal failure, restenoses or stroke, where a therapeutically effective amount of a compound of the general formula (IIA) according to claim 2 is used.

13. A pharmaceutical product comprising a compound of the general formula (IIA) according to claim 2, and usual excipients.

14. A pharmaceutical combination in the form of a product or of a kit composed of individual components consisting a) of a compound of the general formula (IIA) according to claim 2, and b) at least one pharmaceutical form whose active ingredient has a cardiovascular effect.
